(19) United States
(12) Patent Application Publication
(10) Pub. No.: US 2019/0355459 A1
(43) Pub. Date: Nov. 21, 2019

(54) METHODS FOR ADAPTIVE DESIGN OF A TREATMENT REGIMEN AND RELATED TREATMENTS

(71) Applicants: Melanie QUINTANA, Austin, TX (US); Jason CONNOR, Austin, TX (US); Juno Therapeutics, Inc., Seattle, WA (US)

(72) Inventors: He LI, Seattle, WA (US); Tina ALBERTSON, Seattle, WA (US); Melanie QUINTANA, Austin, TX (US); Jason CONNOR, Austin, TX (US)

(73) Assignee: Juno Therapeutics, Inc., Seattle, WA (US)

(21) Appl. No.: 16/085,260

(22) PCT Filed: Mar. 16, 2017
(86) PCT No.: PCT/US2017/022849

§ 371 Date: Sep. 14, 2018

Related U.S. Application Data
(60) Provisional application No. 62/309,442, filed on Mar. 16, 2016.

(51) Int. Cl. G16H 20/10; G16H 50/20

(52) U.S. Cl. CPC G16H 20/10; G16H 50/30; G16H 50/20

(57) ABSTRACT

Provided herein are methods for adaptive design of a treatment regimen for treatment of subjects with a therapeutic agent. In some embodiments, the methods can determine the optimal dosing regimen, such as dose level and/or schedule to administer to a subject. In some embodiments, the methods can be used for determining dosing regimens in clinical trials, such as Phase I clinical trials. In some embodiments, one or more of or all of the steps of the method occur at an electronic device containing one or more processors and memory, such as implemented by a computer. Also provided are methods of administering a therapeutic agent to a subject in accord with an adaptive dosing regimen designed to identify an optimal dose and/or schedule of a therapeutic agent for treating a disease or condition.

### CROSS-REFERENCE TO RELATED APPLICATIONS

This application is a U.S. National Stage of International Application No. PCT/US2017/022849 filed Mar. 16, 2017, which claims priority from U.S. provisional application No. 62/309,442 filed Mar. 16, 2016, entitled “Methods for Adaptive Design of a Treatment Regimen and Related Treatments,” the contents of which is incorporated by reference in its entirety.

### FIELD

The present disclosure relates to methods for adaptive design of a treatment regimen for treatment of subjects with a therapeutic agent. In some embodiments, the methods can determine the optimal dosing regimen, such as dose level and/or schedule to administer to a subject. In some embodiments, the methods can be used for determining dosing regimens in clinical trials, such as Phase I clinical trials. In some embodiments, one or more of or all of the steps of the method occur at an electronic device containing one or more processors and memory, such as implemented by a computer. The present disclosure also relates to methods of administering a therapeutic agent to a subject in accord with an adaptive dosing regimen designed to identify an optimal dose and/or schedule of a therapeutic agent for treating a disease or condition.

### BACKGROUND

Various methods are available for designing clinical trials, including phase I clinical trials. In general, in most methods for the design of phase I clinical trials all patients are treated the same and most data are pooled or determined based on toxicity determination only. Improved methods are needed, for example, to simplify, shorten and/or improve the robustness of the clinical trial. Provided are methods that meet such needs.

### SUMMARY

Provided herein are methods for adaptive allocation of a subject to a treatment regimen for administering a therapeutic agent in a clinical trial, including designating two or more possible unique treatment regimens for administering a therapeutic agent to a population of subjects enrolled in a clinical trial, which population of subjects contain two or more diseases, wherein each of the treatment regimens differ in one or both of a dose level and a schedule, calculating an overall disease-specific utility score for each disease cohort for each treatment regimen, wherein the overall disease-specific utility score is based on response information of subjects within each disease cohort previously treated with the therapeutic agent according to each treatment regimen and on toxicity information of subjects across all disease cohorts previously treated with the therapeutic agent according to each treatment regimen; and allocating the subject to a treatment regimen based on the overall disease cohort utility score.

In some embodiments, the method includes allocating the subjects to a treatment regimen is further based on the number of subjects already allocated to one or more of the treatment regimens or the number of open spots in each treatment regimen. In some embodiments, after allocating the subject to the regimen, the method further includes administering to the subject the therapeutic agent according to the treatment regimen in which the subject has been allocated.

Also provided herein are methods for adaptive treatment of a subject in a clinical trial, including designating two or more possible unique treatment regimens for administering a therapeutic agent to a population of subjects enrolled in a clinical trial, which population of subjects contains two or more diseases, wherein each of the treatment regimens differ in one or both of a dose level and a schedule; calculating an overall disease-specific utility score for each disease cohort for each treatment regimen, wherein the overall disease-specific utility score is based on response information of subjects within each disease cohort previously treated with the therapeutic agent according to each treatment regimen and on toxicity information of subjects across all disease cohorts previously treated with the therapeutic agent according to each treatment regimen; allocating a subject to a treatment regimen based on the overall disease cohort utility score; and administering to the subject the therapeutic agent according to the treatment regimen in which the subject has been allocated.

In some embodiments, allocating the subjects to a treatment regimen is further based on the number of subjects already allocated to one or more of the treatment regimens or the number of open spots in each treatment regimen.

In some embodiments, at least two of the two or more possible unique regimens differ in the dose level and the schedule. In some embodiments, the two or more diseases include two or more disease subtypes.

In some embodiments, calculating the overall disease-specific utility score for each disease cohort for each treatment regimen includes i) determining a toxicity rate based on the toxicity information of subjects previously treated across all disease cohorts for each treatment regimen; ii) determining an efficacy rate based on the response information of subjects previously treated within each disease cohort for each treatment regimen; iii) generating a safety utility score as a function of the toxicity rate for the treatment regimen; iv) generating an efficacy utility score as a function of the efficacy rate for the treatment regimen; and v) multiplying the safety utility score by the efficacy utility score, thereby calculating the overall disease-specific utility score for each disease cohort for each treatment regimen.

In some embodiments, the method further includes reporting the overall disease-specific utility score.

In some embodiments of the method, in step iii), if the toxicity rate is determined to be less than or equal to a utility safety target, then the function defines the safety utility score as 1; if the toxicity rate is determined to be greater or equal to a utility safety limit, then the function defines the safety utility score as 0; and if the toxicity rate is projected to be between the utility safety target and the utility safety limit, then the safety utility score decreases linearly as the toxicity rate increases.

In some embodiments of the method, in step iv), if the efficacy rate is projected to be less than or equal to a utility efficacy target, then the function defines the efficacy utility score as 0; if the efficacy rate is projected to be greater than the utility efficacy target, then the efficacy utility score increases linearly as the efficacy rate increases.

In some embodiments, the toxicity rate is a dose-limiting toxicity (DLT) rate. In some embodiments, the response rate, i.e., efficacy rate, is a complete response (CR) rate.

In some embodiments, the toxicity rate is determined by a regimen-toxicity model that estimates a single toxicity rate for each regimen across all disease cohorts. In some embodiments, the regimen-toxicity model contains the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {β}_{3} {1}_{[ s = 2 ⋂ d = - 1 ]}$$

In some embodiments, the efficacy rate is determined by a regimen-response model that estimates a single efficacy rate within each disease cohort for each regimen. In some embodiments, the regimen-response model borrows response information across disease cohorts. In some embodiments, the regimen response model contains the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

In some embodiments, the response information is borrowed across disease cohorts according to the following distribution:

$${α}_{0 , DLBCL} , {α}_{0 , MCL} \text{∼} N ( θ , {σ}^{2} ) ; θ \text{∼} N ( - 0.5 , {4}^{2} ) ; \frac{1}{{σ}^{2}} \text{∼} Gamma ( 2 , 2 ) .$$

In some embodiments, each of the two or more treatment regimens has a first status of either open or closed; and the subject is allocated into an open treatment regimen. In some embodiments, prior to allocating the subject, the method further includes: determining if the first status of any of the treatment regimens should be changed from closed to open; and if the first status of a treatment regimen should be changed, changing the first status to open.

In some embodiments, determining if the first status of a treatment regimen should be changed includes determining if another open regimen is considered to be safe, effective, unsafe, or ineffective. In some embodiments, a treatment regimen is determined to be unsafe for the purpose of opening another regimen probability that the toxicity rate is less than a first open safety limit is less than an open toxicity probability parameter; and/or a treatment regimen is determined to be ineffective for the purpose of opening another regimen the probability that the efficacy rate is greater than a first open efficacy target is less than an open efficacy probability parameter. In some embodiments, the utility and first open safety limits can be the same or different; and/or the first and second efficacy targets can be the same or different.

In some embodiments, a treatment regimen is determined to be safe for the purpose of opening another regimen if the probability that the toxicity rate is less than a second safety limit is greater than a second toxicity probability parameter; and/or a treatment regimen is determined to be effective for the purpose of opening another regimen if the probability that the efficacy rate is greater than a second efficacy target is greater than a second efficacy probability parameter. In some embodiments, any of the utility, first, and second safety targets can be the same or different; and/or any of the utility, first, and second, efficacy targets can be the same or different.

In some embodiments, the status of a first regimen is open, and a second regimen is a de-escalation regimen of the first open regimen. In some embodiments, the status of the second regimen is changed to open when the first regimen is determined to be unsafe for the purpose of opening another regimen.

In some embodiments, the status of the first regimen is open, and a third regimen is an escalation regimen of the first open regimen. In some embodiments, the status of the third regimen is changed to open when the first regimen is determined to be ineffective for the purpose of opening another regimen. In some embodiments, the status of the third regimen is changed to open when the first regimen is determined to be safe for the purpose of opening another regimen. In some embodiments, the status of the second regimen is open and the status of the third regimen is open, and a fourth regimen is an escalation of the second regimen and a de-escalation of the third regimen.

In some embodiments, the status of the fourth regimen is changed to open when: the first regimen is determined to be safe for the purpose of opening another regimen and the third regimen is determined to be unsafe for the purpose of opening another regimen; or the second regimen is determined to be safe for the purpose of opening another regimen and the fourth regimen is determined to be safe for the purpose of opening another regimen across disease cohorts.

In some embodiments, a regimen with a first status of open includes a second status, wherein the second status can be either eligible or suspended. In some embodiments, the second status is assigned to be eligible when the first status is changed to open.

In some embodiments, the second status is temporarily changed to suspended: if the probability that the toxicity rate is less than a third safety limit is less than a third toxicity probability parameter; and/or if the probability that the efficacy rate is greater than a third efficacy target is less than a third efficacy probability parameter. In some embodiments, any of the utility, first, second, and third safety targets can be the same or different; and/or any of the utility, first, second, and third efficacy targets can be the same or different.

In some embodiments, a treatment regimen is determined to be safe for the purpose of suspending the regimen if the probability that the toxicity rate is less than a fourth safety limit is greater than a fourth toxicity probability parameter; and/or the treatment regimen is determined to be effective for the purpose of suspending the regimen if the probability that the efficacy rate is greater than a fourth efficacy target is greater than a fourth efficacy probability parameter.

In some embodiments, any of the utility, first, second, third, and fourth safety targets can be the same or different; and/or any of the utility, first, second, third, and fourth efficacy targets can be the same or different.

In some embodiments, allocating the subject to a treatment regimen is further based on the relative uncertainty of the estimate for the utility of each regimen. In some embodiments, allocating a subject to a treatment regimen includes a randomization probability that a subject will be enrolled in each of the regimen within each disease cohort with a first status of open and a second status of eligible, wherein the randomization probability V_{d,s,h} is:

$${V}_{d , s , h} ∝ {[ \frac{Pr ( {r}_{d , s , h} = {r}_{{d}^{*} , {s}^{*} , h} ) Var ( {U}_{d , s . h} )}{{n}_{d , s , h} + 1} ]}^{\frac{1}{2}} .$$

wherein r_{d,s,h} is the regimen of dose level d, schedule s, and disease h, Pr(r_{d,s,h}=r_{d*,s*,h}) is the probability the regimen is the highest utility regimen, Var(U_{d,s,h}) is the variance of the regimen's disease-specific utility score, and n_{d,s,h} is the number of subjects already allocated to the regimen within disease cohort h.

In some embodiments, the clinical trial ends if: none of the regimens have a first status of open and a second status of eligible; or a total number of enrolled subjects is equal to or greater than a maximum subject enrollment limit.

In some embodiments, the clinical trial ends for one of the two or more diseases if: none of the regimens for the disease have a first status of open and a second status of eligible; a total number of enrolled subjects with the disease is equal to or greater than a maximum disease-subject enrollment limit; response and toxicity information within the disease cohort has been determined for a first pre-determined minimum number of subjects within the regimen with the highest utility and the regimen is unlikely to be effective within the cohort for the purpose of termination; or response and toxicity information within the disease cohort has been determined for a second pre-determined minimum number of subjects and the regimen with the highest utility is highly likely to be safe and effective within the cohort for the purpose of termination.

In some embodiments, a regimen is unlikely to be effective within the cohort for the purpose of termination if the probability that the efficacy rate is greater than a termination efficacy target is less than a termination efficacy probability parameter. In some embodiments, a regimen is unlikely to be safe across the cohort for the purpose of termination if the probability that the toxicity rate is less than a termination safety limit is less than a termination toxicity probability parameter.

In some embodiments, a regimen is likely to be safe and effective within the cohort for the purpose of termination for early success if: the probability that the toxicity rate is less than a success safety limit is greater than a success toxicity probability parameter; and the probability that the efficacy rate is greater than a success efficacy target is greater than a success efficacy probability parameter.

In some embodiments, the therapeutic agent is one in which the response information can be determined within the same period of time in which the toxicity information is determined. In some embodiments, the therapeutic agent includes an adoptive cell therapy, a small molecule, a gene therapy, or a transplant. In some embodiments, the therapeutic agent includes cells expressing a chimeric antigen receptor (CAR). In some embodiments, the CAR expressed by the cells specifically binds to an antigen expressed by a cell or tissue of at least one of the two or more diseases or associated with at least one of the two or more diseases.

In some embodiments, at least one of the two or more diseases is a tumor or a cancer. In some embodiments, at least one of the two or more diseases is a leukemia or lymphoma. In some embodiments, at least one of the two or more diseases is acute lymphoblastic leukemia. In some embodiments, at least one of the two or more diseases is a non-Hodgkin lymphoma (NHL). In some embodiments, at least one of the two or more diseases is diffuse large B cell lymphoma (DLBCL). In some embodiments, at least one of the two or more diseases is mantle cell lymphoma (MCL).

In some embodiments, the number of cells administered in the first dose is between about 0.5×10⁶ cells/kg body weight of the subject and 6×10⁶ cells/kg, between about 0.75×10⁶ cells/kg and 2.5×10⁶ cells/kg, between about 1×10⁶ cells/kg and 2×10⁶ cells/kg, between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive.

In some embodiments, the dose of cells is administered in a single pharmaceutical composition containing the cells of the dose. In some embodiments, the dose is a split dose, wherein the cells of the dose are administered in a plurality of compositions, collectively contains the cells of the dose, over a period of no more than three days.

In some embodiments, the method is repeated for one or more additional subjects enrolled in the clinical trial. In some embodiments, the clinical trial is a phase I clinical trial.

Also provided herein are methods of calculating an overall disease-specific utility score for each disease cohort for each treatment regimen in a clinical trial, including: a) determining a toxicity rate based on the toxicity information of subjects previously treated across all disease cohorts for each treatment regimen; b) determining an efficacy rate based on the response information of subjects previously treated within each disease cohort for each treatment regimen; c) generating a safety utility score as a function of the toxicity rate for the treatment regimen; d) generating an efficacy utility score as a function of the efficacy rate for the treatment regimen; and e) multiplying the safety utility score by the efficacy utility score, thereby calculating the overall disease-specific utility score for each disease cohort for each treatment regimen.

In some embodiments, the method further includes reporting the overall disease-specific utility score for each disease cohort for each treatment regimen. In some embodiments, if the toxicity rate is determined to be less than or equal to a first safety target, then the function defines the safety utility score as 1; if the toxicity rate is determined to be greater or equal to a first safety limit, then the function defines the safety utility score as 0; and if the toxicity rate is projected to be between the first safety target and the first safety limit, then the safety utility score decreases linearly as the toxicity rate increases.

In some embodiments, if the efficacy rate is projected to be less than or equal to an efficacy target, then the function defines the efficacy utility score as 0; if the efficacy rate is projected to be greater than the efficacy target, then the efficacy utility score increases linearly as the efficacy rate increases.

In some embodiments, the toxicity rate is a dose-limiting toxicity (DLT) rate. In some embodiments, the response rate, i.e., the efficacy rate, is a complete response (CR) rate.

In some embodiments, the toxicity rate is determined by a regimen-toxicity model that a single toxicity rate for each regimen across all disease cohorts. In some embodiments, the regimen-toxicity model contains the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {β}_{3} {1}_{[ s = 2 ⋂ d = - 1 ]}$$

In some embodiments, the efficacy rate is determined by a regimen-response model that estimates a single efficacy rate within each disease cohort for each regimen. In some embodiments, the regimen-response model borrows response information across disease cohorts. In some embodiments, the regimen response model contains the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

In some embodiments, the response information is borrowed across disease cohorts according to the following distribution:

$${α}_{0 , DLBCL} , {α}_{0 , MCL} \text{∼} N ( θ , {σ}^{2} ) ; θ \text{∼} N ( - 0.5 , {4}^{2} ) ; \frac{1}{{σ}^{2}} \text{∼} Gamma ( 2 , 2 ) .$$

In some embodiments, the subject is a human subject.

In some embodiments, the method is a computer implemented method, wherein one or more steps a)-c) occur at an electronic device containing one or more processors and memory.

Also provided herein is a computer system containing a processor and memory, the memory containing instructions operable to cause the processor to carry out any one or more of steps a)-c) of any of the provided methods.

In some embodiments, the method is a computer implemented method, wherein one or more steps of any of the provided methods occur at an electronic device containing one or more processors and memory.

Also provided herein is a computer system containing a processor and memory, the memory containing instructions operable to cause the processor to carry out any one or more of steps of any of the provided methods.

### BRIEF DESCRIPTION OF THE DRAWINGS

FIG. 1 shows an exemplary safety utility function (left panel) and efficacy utility function (right panel), as described in Example 1. The utility score is a function of the probability of a safety outcome (left panel) or an efficacy outcome (right panel). In this example, safety outcomes are dose limiting toxicity outcomes, and efficacy outcomes are complete response outcomes.

FIG. 2A shows the results of the first interim analysis of a simulated clinical trial as described in Example 2. In the “Safety Model+Allocation” and “Efficacy Model+Allocation” plots, open circles represent the probability (corresponding to the left y-axis) of DLT and CR rate estimated from the model with 95% confidence interval (line) and solid circle represents the observed rates; the bar plot shows sample size in each group where the height (corresponding to the right y-axis) of the solid bar shows number of enrolled patients and height of the greyed bar show number of DLT and CR evaluable patients. In the four middle plots, the solid bar represents the open dosed cohorts with estimated posterior probability. The “Utility” plot represents the estimated utility and the “Allo. Prob.” shows the allocation/randomization probability to the open regimen. In the efficacy, utility and allocation chart, the left bar on a regimen represents DLBCL group and the right bar represents MCL group. Of all open regimens, the one with the highest utility is shaded dark.

FIG. 2B shows the results of the second interim analysis of the simulated clinical trial described in Example 2. At this stage, 15 (12 DLBCL and 3 MCL) patients were enrolled to Dose Level 1, single-dose schedule; 10 DLBCL and 3 MCL patients completed the DLT and efficacy evaluation period. Two DLTs; 6 DLBCL achieve CR and zero MCL patient achieve CR. All regimens are likely to be safe at this stage. Dose Level 1, 2-dose was open for both disease cohorts.

FIG. 2C shows the results of the third interim analysis of the simulated clinical trial described in Example 2. At this stage, 18 (12 DLBCL and 6 MCL) patients completed safety and efficacy evaluation period at dose level 1, single-dose schedule; at this regimen, 2 patients experience DLT, 7 DLBCL patients achieve CR and one MCL patient achieves CR. Eighteen (14 DLBCL and 4 MCL) patients completed evaluation period at dose level 1, 2-dose schedule; at this regimen, 4 patients experienced DLT, 9 DLBCL patients achieved CR and one MCL patient achieved CR. All regimens are likely to be safe at this stage. The trial stopped enrolling DLBCL due to early success. Dose level 1, single-dose schedule was suspended for enrollment for MCL due to futility. The trial continued to enroll MCL patients at dose level 1, 2-dose schedule.

FIG. 2D shows the results of the final analysis for the completed trial in the simulated clinical trial described in Example 2. At this stage, an additional 8 MCL patients completed dose level 1, 2-dose schedule; at this regimen, 4 patients experienced DLT, and 6 MCL patients achieved CR. The trial will stop early due to success.

FIGS. 3A-3D show graphs displaying the operating characteristics for scenarios 1.1 (FIG. 3A), 2.1 (FIG. 3B), 3.1 (FIG. 3A), and 4.1 (FIG. 3D) as described in the Examples for DLBCL (D) and MCL (M) disease cohorts. The graphs on the left display the probability that each regimen is open at some point during the trial for each disease cohort. The graphs on the left also display the CR rates for the DLBCL and MCL disease cohorts as indicated by the “*” in the middle of the DLBCL and MCL columns, respectively. The DLT rate for each scenario is indicated by an “*” that is placed between the DLBCL and MCL columns. The middle graphs display the average sample size on each regimen. The graphs on the right display the probability that each regimen is determined to be the highest utility regimen and to be safe and effective, as well as the probability that no regimen is determined to be safe and effective.

FIGS. 4A-D show graphs displaying the operating characteristics for scenarios 1.2 (FIG. 4A), 2.2 (FIG. 4B), 3.2 (FIG. 4C), and 4.2 (FIG. 4D) as described in the Examples for DLBCL (D) and MCL (M) disease cohorts. The graphs on the left display the probability that each regimen is open at some point during the trial for each disease cohort. The graphs on the left also display the CR rates for the DLBCL and MCL disease cohorts as indicated by the “*” in the middle of the DLBCL and MCL columns, respectively. The DLT rate for each scenario is indicated by an “*” that is placed between the DLBCL and MCL columns. The middle graphs display the average sample size on each regimen. The graphs on the right display the probability that each regimen is determined to be the highest utility regimen and to be safe and effective, as well as the probability that no regimen is determined to be safe and effective.

FIGS. 5A-D show graphs displaying the operating characteristics for scenarios 1.3 (FIG. 5A), 2.3 (FIG. 5B), 3.3 (FIG. 5C), and 4.3 (FIG. 5D) as described in the Examples for the DLBCL (D) and MCL (M) disease cohorts. The graphs on the left display the probability that each regimen is open at some point during the trial for each disease cohort. The graphs on the left also display the CR rates for the DLBCL and MCL disease cohorts as indicated by the “*” in the middle of the DLBCL and MCL column, respectively. The DLT rate for each scenario is indicated by an “*” that is placed between the DLBCL and MCL columns. The middle graphs display the average sample size on each regimen. The graphs on the right display the probability that each regimen is determined to be the highest utility regimen and to be safe and effective, as well as the probability that no regimen is determined to be safe and effective.

### DETAILED DESCRIPTION

Provided herein are methods for allocating subjects to a treatment regimen. Is some embodiments, the treatment regimen is part of a clinical trial. In some embodiments, the subjects are patients, including human patients.

### I. OVERVIEW

Provided herein are methods to determine the optimal dosing regimen of a therapeutic agent. In some embodiments, the methods are designed to determine the optimal dosing regimen in a mix patient population. In some embodiments, such methods can be used in the design of treatment regimens, such as in clinical trials, in which one or more diseases or one or more disease subtypes may be present and treatable with the same therapeutic agent. As an example, the presence of mixed populations of subjects can exist in methods of treating B-cell Non-Hodgkin Lymphoma, such as by adoptive cell therapies. In contrast, many existing methods all patients are treated the same regardless of underlying heterogeneity, e.g. disease subtype, and all data are simply pooled for the MTD determination. This may estimate the dose-toxicity relationship for the whole population, but may not accurately estimate the MTD for a particular disease subtype. Further, in some cases, conducting clinical trials across different patient populations to determine MTD can be inefficient.

The provided methods address these limitations. The provided approach involves enrolling multiple patient populations into the same trial. In the provided design, the method allows borrowing of information from safety or efficacy data using Bayesian hierarchical modeling.

In some embodiments, the provided methods are also based on considering both safety and efficacy information in the clinical design. In general, Phase 1 oncology trials generally test an experimental drug at a fixed sequence of dose levels with a single schedule of administration, and then aim to identify the maximum tolerated dose (MTD). In some cases, such methods use rule-based designs or model-based designs in which the dose-limiting toxicity (DLT) data only is considered and assume a monotone dose-response relationship. This may be a reasonable assumption for cytotoxic agents, however, for molecular targeted agents or immunotherapies, this may not be appropriate. For certain clinical data, a range of dose levels is generally safe and effective, and hence, dose escalation beyond this range of dose levels may be unnecessary. Thus, it is found herein that a dosing regimen strategy can be optimized or improved by combining safety and efficacy data.

Generally, Phase I clinical trials are designed to determine if a therapeutic is safe at various dosing levels for further study. The therapeutic is administered to subjects, who are then observed for a period of time. Trial design is frequently “fixed.” Decisions regarding key trial parameters are traditionally defined before the study begins and then left unchanged during the trial itself. This is true for some trials even when initial clinical data analyzed at interim points of a trial suggest that treatment regimens should be modified.

Adaptive clinical trials are designed to take advantage of this accumulating information, by allowing modification to key trial parameters in response to accumulating information and according to predefined rules. This can be especially powerful when outcomes can be determined rapidly relative to the time required for an entire trial. Adaptive clinical trials incorporate frequent interim analyses of the data as they becomes available, and use the data to direct future subjects towards treatment regimens that reduce overall uncertainty and provide the highest likelihood of success.

Additionally, some experimental therapeutics are capable of yielding a therapeutic response during the period of time necessary to determine safety. Thus, in some embodiments, by simultaneously evaluating and adaptively optimizing a therapeutic regimen for both safety and efficacy, the study can increase the likelihood of matching subjects to the treatments most likely to be safe and beneficial while reducing the number of subjects allocated to unsafe or ineffective regimens.

In some embodiments, for example, complete responses (CRs) to CAR-T therapy and other molecular targeted or immunotherapies can appear rapidly. In some cases, this makes it possible to jointly assess safety and toxicity in the same timeframe, and hence provides an opportunity to jointly assess them for the optimal regimen determination. In some cases, the model is designed with the consideration that some disease subtypes may exhibits the same dose-toxicity curve but may have different response curves. Thus, in some embodiments, to guide the regimen selection, toxicity information is pooled across disease subtypes using a single regimen-toxcity model, whereas response information can be modelled separately and information shared between diseases. In some embodiments, this design allows for disease-specific estimates of efficacy.

In some embodiments, Bayesian clinical trial designs with hierarchical borrowing can yield similarly small error rates as larger, traditional clinical trial designs, but with increased power and reduced mean sample sizes. In some embodiments, Bayesian clinical trials can also be more likely to make correct safety and/or efficacy conclusions regarding a therapeutic than other clinical trial designs in many circumstances.

In some embodiments, methods are provided that employ a Bayesian adaptive design to address the challenging and practical aspects of enrolling a clinical trial with multiple dosing regimens and multiple disease subtypes. In certain embodiments, an advantage of the methods presented herein is that they are flexible and demonstrate good operating characteristics through simulation studies of diverse clinical scenarios. While in some embodiments, the methods provided herein are applied in the setting of a mixed population, e.g., a population of subjects with two or more diseases or disease subtypes, the methods may also be applied to a homogenous population of subjects with only one disease or disease subtype. In some embodiments, the methods provided herein may be applied to a population of subjects having one disease differentiated by one or more genetic abnormalities or differences in one or more biomarkers.

In particular embodiments, the provided methods reduce the number of patients treated at sub-therapeutic dose levels while preserving safety and maintaining rapid accrual in phase 1 dose-escalation trials. In some embodiments, the open enrollment and eligible for enrollment features of the methods provided herein possess an advantage in that patients can be enrolled as they become available with fewer pauses in enrollment. Furthermore, in some embodiments, additional rules have been introduced that provide adequate safety measures to the methods provided herein. For example, in some embodiments, at least 3 subjects with complete DLT information at the starting dose are required before proposing to escalate and escalation only takes place from adjacent doses. In some embodiments, a 2-dose schedule of each dose is only opened if there are at least 3 subjects with complete DLT information on the single-dose schedule of that dose. In certain embodiments, the methods comprise restrictions on the maximum queue size (i.e., the maximum number of subjects with unknown DLT information). In addition, in some embodiments, if at any point in the trial a regimen is estimated to have a DLT rate that is greater than 33%, enrollment to the regimen is turned off.

Particular embodiments contemplate that traditional methods for evaluating a regimen in a Phase I clinical study would not take efficacy into account, and do not have rules, e.g., rules for opening, closing, or assigning subjects to a regimen, beyond 6 treated subjects. In some embodiments, the methods provided herein are more able to accurately identify optimal doses of a therapeutic agent based on safety and efficacy than the traditional methods.

In some embodiments, methods provided herein comprise features that include the use of a utility score that is based on safety and efficacy data that guide regimen selection, and an adaptive randomization approach to enroll multiple regimens that are open and eligible for enrollment. In certain embodiments, the randomization probability is based on the trade-off between efficacy and toxicity. The ability to use both safety and efficacy outcomes in this way depends on the fact that with some therapeutic agents, e.g., a CAR-T therapy, the best response is generally observed in the same timeframe as the safety endpoint. In particular embodiments, other data such as cell expansion, biomarker, and immunogenicity may also be considered for the interim dose escalation/de-escalation decision and final regimen selection. In certain embodiments, the provided methods improve over the traditional phase 1 design, for example by minimizing both the over- and under-dosing risk to patients while maximizing efficiency. In particular embodiments, the methods provided herein not only inform on the optimal dosing strategy, but also inform on a potential patient population for the future phase 2 and 3 studies.

### II. METHODS FOR ALLOCATING SUBJECTS TO A TREATMENT REGIMEN

Provided herein are methods for adaptive allocation of a subject to a treatment regimen for administering a therapeutic agent in a clinical trial. Generally, the clinical trial will analyze the safety and/or efficacy of a therapeutic agent in treating one or more diseases or conditions, such as treating two or more diseases or conditions. In some embodiments, the two or more diseases or conditions can be different diseases or conditions, or sub-types of the same disease or condition. Generally, entering subjects will be classified as having one of the one or more diseases, conditions, or subtypes before being assigned to a regimen.

In certain embodiments, methods are provided for the adaptive allocation of subjects from one disease population or cohort to a treatment regimen for administering a therapeutic agent, for example in a clinical trial, such as a Phase I clinical trial. In particular embodiments, methods are provided for the adaptive allocation of subjects from two or more disease populations or cohorts to a treatment regimen for administering a therapeutic agent in a clinical trial.

In particular embodiments, a population of subjects are allocated to treatment regimens, for example to evaluate different treatment regimens in a clinical trial. In certain embodiments, the population of subjects comprises subjects from more than one disease cohort. In some embodiments, a disease cohort is defined by the identity of the disease. In particular embodiments, a disease cohort is defined by any characteristic of the same disease that might affect the efficacy of a therapeutic agent. In some embodiments, subjects from different disease cohorts have different diseases. In certain embodiments, subjects from different disease cohorts have different diseases that are caused by, generated by, exacerbated by, and/or associated with one or more of the same therapeutic targets (e.g., a therapeutic target molecule and/or an antigen). In certain embodiments, subjects from different disease cohorts have the same disease but different subtypes of the same disease. In some embodiments, subjects from different disease cohorts have the same disease but with one or more different characteristics. Characteristics of the disease may include, but are not limited to, prior treatment history, age of onset, localization (e.g., location of a primary tumor), severity, progression, symptoms, pathology, mutation, and/or etiology. In some embodiments, subjects from different disease cohorts have the same disease, i.e., the disease to be treated by the therapeutic agent, but subjects from at least one disease cohort have an additional disease. In particular embodiments, subjects from different disease cohorts have the same disease, i.e., the disease to be treated by the therapeutic agent, but have different additional diseases.

In some embodiments, the subjects have a cancer or proliferative disease that is a B cell malignancy or hematological malignancy. In certain embodiments, at least one disease cohort contains subjects with a cancer or proliferative disease. In particular embodiments, the cancer or proliferative disease is multiple myeloma (MM), acute lymphoblastic leukemia (ALL), non-Hodgkin's lymphoma (NHL), chronic lymphocytic leukemia (CLL), an acute lymphoblastic leukemia (ALL), mantle cell lymphoma (MCL), a diffuse large B-cell lymphoma (DLBCL), or acute myeloid leukemia (AML). In particular embodiments, a population of subjects has NHL, and a cohort, e.g., a disease cohort, of subjects within the population has DBCBL. In certain embodiments, a population of subjects has NHL, and a cohort of subjects within the population has MCL. In some embodiments, at least one disease cohort comprises subjects with DBCBL. In particular embodiments, at least one disease cohort comprises subjects with MCL.

In certain embodiments, one or more cohorts may be determined based on one or a plurality of genetic mutations and/or abnormalities associated with the disease or condition and/or the treatment regimen. In some embodiments, one or more cohorts may be determined based upon the presence or absence of a biomarker of a particular disease stage, disease subgroup, likelihood of responding to treatment, genetic abnormality, or other biomarker. For example, genetic abnormalities of B-cell leukemia, for example CLL, include complex karyotype, del(17p), trisomy of chromosome 12, deletion of chromosome 13 (13q14), chromosome 14 abnormalities (e.g. t(11;14)(q13;q32)), deletions of the long arm of chromosome 18 (18q21)(q32;q13.1), and aberrations in chromosome 17 and the p53 mutations (17p13.1). In some embodiments, the genetic abnormality includes translocation between chromosomes 8 and 21, translocation or inversion in chromosome 16, changes in chromosome 11, and (M3), which in some cases has translocation between chromosomes 15 and 17. In certain embodiments, patients may be grouped into cohorts based upon one or more non-genetic factors such as quantity and type of previous treatment and/or intervention. In some specific embodiments, one or more cohorts of patients may present the same disease, but may differ in one or more other parameters, such as genetic abnormalities and/or treatment history. In some embodiments, the cohorts are determined based upon treatment history.

In some embodiments, one or more cohorts may be determined based on grade and/or stage of disease. For example, low grade (indolent), intermediate, or high grade (aggressive) lymphoma. Further examples include early pre-B ALL, common ALL, pre-B ALL, mature B-cell ALL (Burkitt leukemia), Undifferentiated AML—M0, Myeloblastic leukemia—M1, Myeloblastic leukemia—M2, Promyelocytic leukemia—M3, Myelomonocytic leukemia—M4, Monocytic leukemia—M5, Erythroleukemia—M6, and Megakaryoblastic leukemia—M7. Further examples include, Rai Stage 0 CLL, Rai Stage I CLL, Rai Stage II CLL, Rai Stage III CLL, Rai Stage IV CLL, or using the Binet staging system for CLL: Clinical stage A, Clinical stage B, or Clinical stage C. Examples of chronic myeloid leukemia (CML) stages include chronic, accelerated, and blastic. Examples of myeloma stages include stages I, II, and III based on the Dune-Salmon staging system, and stages I, II, and III, based on the International Staging System.

In certain embodiments, the methods comprise designating two or more possible unique treatment regimens for administering a therapeutic agent to a population of subjects enrolled in a clinical trial. In some embodiments, the regimens differ by the number of therapeutic agents administered, the amount of a dose of a therapeutic agent administered, the number of therapeutic doses administered, the route of administration, e.g., intravenous, oral, etc., the timing and/or dosing schedule, e.g., the amount of time between different doses, and/or formulations of the doses containing the therapeutic agent. In certain embodiments, the clinical trial comprises regimens with two, three, four, five, six, seven, eight, nine, ten, eleven, twelve, thirteen, fourteen, fifteen, sixteen, seventeen, eighteen, nineteen, twenty, or more than twenty different amounts of the dose. In particular embodiments, the clinical trial comprises regimens with two, three, four, five, six, seven, eight, nine, ten, eleven, twelve, thirteen, fourteen, fifteen, sixteen, seventeen, eighteen, nineteen, twenty, or more than twenty different variations in the number or frequency of doses. In some embodiments, the clinical trial comprises regimens with two, three, four, five, six, seven, eight, nine, ten, eleven, twelve, thirteen, fourteen, fifteen, sixteen, seventeen, eighteen, nineteen, twenty, or more than twenty different variations in the timing of doses.

In some embodiments, the methods comprise designating two or more possible unique treatment regimens for administering a therapeutic agent to a population of subjects enrolled in a clinical trial. Generally, the clinical trial will comprise two or more possible unique regimens for administering a therapeutic agent to a population of subjects. The regimens can differ by one or more of a number of possible variables. Such variables can include, for example, dose and schedule (e.g., the number of doses and/or the timing of administration). In some embodiments, the regimens all involve the same number and timing of doses, but vary in the amount of the dose. In some embodiments, the regimens involve the same amount and timing of doses, but vary as to the number of doses. In some embodiments, the regimens vary by timing of dose, but have the same number and amount. In some embodiments, the regimens vary by timing and amount of the dose, but have the same number of doses. In some embodiments, the regimens vary by number and amount of the dose, but have the same timing. In some embodiments, the regimens vary by number of doses and timing, but have the same amount of the dose. In some embodiments, the regimens vary by number of doses, timing, and amount of the dose. In some embodiments, one or more regimens can include the administration of three or more doses. In some embodiments, the timing between each dose can be the same or different, and the amount of each dose can be the same or different.

In particular embodiments, the methods comprise designating two, three, four, five, six, seven, eight, nine, ten, eleven, twelve, thirteen, fourteen, fifteen, sixteen, seventeen, eighteen, nineteen, twenty, or more than twenty different treatment regimens. In certain embodiments, methods are provided for the adaptive allocation of subjects from one, two, three, four, five, six, seven, eight, nine, ten, eleven, twelve, thirteen, fourteen, fifteen, sixteen, seventeen, eighteen, nineteen, twenty, or more than twenty disease cohorts to a treatment regimen.

In some embodiments, the method further comprises administering to the subject the therapeutic agent according to the treatment regimen in which the subject has been allocated.

Generally, the regimens will include an initial regimen to which initial subjects are enrolled. In some embodiments, one or more of the other doses will be an escalation of the initial regimen. In some embodiments, one or more of the other doses will be a de-escalation. In some embodiments, an escalation can be an escalation in dose amount, number of doses, timing, or a combination of any of these variables. In some embodiments, a de-escalation can be a de-escalation in dose amount, number of doses, timing, or a combination of any of these variables. In some embodiments, a first regimen will be an escalation of a second regimen. In some embodiments, a first regimen will be a de-escalation of a second regimen. In some embodiments, a first regimen will have a second regimen that is an escalation relative to the first regimen. In some embodiments, a first regimen will have a second regimen that is a de-escalation relative to the first regimen. In some embodiments, a first regimen will have a second regimen that is an escalation relative to the first regimen, and a third regimen that is a de-escalation relative to the first regimen.

In some embodiments, the amount or quantity of a dose of a therapeutic agent may refer to, in the exemplary case of modified T cells, the quantity of total T cells, quantity of modified T cells, quantity of total CD4+ cells, quantity of total CD8+ cells, quantity of total CD4+ modified T cells, and/or quantity of total CD8+ modified T cells, or any combinations thereof. In some embodiments, the amount or quantity of a dose of a therapeutic agent may refer to, in the exemplary case of modified T cells, the quantity of T cells per kg, quantity of CAR+ T cells per kg, quantity of CD4+ cells per kg, quantity of CD8+ cells per kg, quantity of CD4+ modified T cells per kg, and/or quantity of CD8+ modified T cells per kg, or any combinations thereof. In some embodiments, the amount or quantity of a dose of a therapeutic agent may refer to, in the exemplary case of CAR-modified T cells, the quantity of total T cells, quantity of CAR+ T cells, quantity of total CD4+ cells, quantity of total CD8+ cells, quantity of total CD4+ CAR+ T cells, and/or quantity of total CD8+ CAR+ T cells, or any combinations thereof. In some embodiments, the amount or quantity of a dose of a therapeutic agent may refer to, in the exemplary case of CAR-modified T cells, the quantity of T cells per kg, quantity of CAR+ T cells per kg, quantity of CD4+ cells per kg, quantity of CD8+ cells per kg, quantity of CD4+ CAR+ T cells per kg, and/or quantity of CD8+ CAR+ T cells per kg, or any combinations thereof. In some embodiments, the amount or quantity of a dose of a therapeutic agent may refer to, in the exemplary case of exogenous TCR-modified T cells, the quantity of total T cells, quantity of exogenous TCR+ T cells, quantity of total CD4+ cells, quantity of total CD8+ cells, quantity of total CD4+exogenous TCR+ T cells, and/or quantity of total CD8+exogenous TCR+ T cells, or any combinations thereof. In some embodiments, the amount or quantity of a dose of a therapeutic agent may refer to, in the exemplary case of exogenous TCR-modified T cells, the quantity of T cells per kg, quantity of exogenous TCR+ T cells per kg, quantity of CD4+ cells per kg, quantity of CD8+ cells per kg, quantity of CD4+ exogenous TCR+ T cells per kg, and/or quantity of CD8+ exogenous TCR+ T cells per kg, or any combinations thereof.

In some embodiments, a particular regimen is considered to be an escalation of a different regimen if the particular regimen comprises administering a larger dose of a therapeutic agent than the dose of the different regimen. In certain embodiments, the particular regimen is considered to be an escalation as compared to the different regimen if the amount of the therapeutic agent administered in a dose is at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least about 100% greater than the amount of the therapeutic agent administered in the different regimen. In some embodiments, the particular regimen is considered to be an escalation as compared to the different regimen if the amount of the therapeutic agent administered in a dose is at least about 1-fold, about 1.5 fold, about 2.0-fold, about 2.5-fold, about 3.0-fold, about 3.5-fold, about 4.0-fold, about 4.5-fold, about 5.0-fold, about 6-fold, about 7-fold, about 8-fold, about 9-fold, about 10-fold, about 15-fold, about 20-fold, about 30-fold, about 40-fold, or greater than at least 50-fold greater than the amount of the therapeutic agent administered in the different regimen.

In particular embodiments, a particular regimen is considered to be a de-escalation of a different regimen if the particular regimen comprises administering a smaller dose of a therapeutic agent than the dose of the different regimen. In certain embodiments, the particular regimen is considered to be a de-escalation as compared to the different regimen if the amount of the therapeutic agent administered in a dose that is at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least about 99% less than the amount of the therapeutic agent administered in the different regimen.

In some embodiments, a particular regimen is considered to be an escalation of a different regimen if the particular regimen comprises administering a larger number of doses of the therapeutic agent than the different regimen. In certain embodiments, the particular regimen is considered to be an escalation as compared to the different regimen if the number of the doses of the therapeutic agent is increased by one dose, two doses, three doses, four doses, five doses, six doses, seven doses, eight doses, nine doses, ten doses, or at least ten doses, at least 15 doses, at least 20 doses, at least 25 doses, at least 30 doses, at least 35 doses, at least 40 doses, at least 45 doses, or at least 50 doses. In some embodiments, the particular regimen is considered to be an escalation as compared to the different regimen if the number of doses of the therapeutic agent administered is increased by at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least about 100% as compared to the different regimen. In some embodiments, the particular regimen is considered to be an escalation as compared to the different regimen if the number of doses the therapeutic agent is increased by at least about 1-fold, about 1.5 fold, about 2.0-fold, about 2.5-fold, about 3.0-fold, about 3.5-fold, about 4.0-fold, about 4.5-fold, about 5.0-fold, about 6-fold, about 7-fold, about 8-fold, about 9-fold, about 10-fold, about 15-fold, about 20-fold, about 30-fold, about 40-fold, or greater than at least 50-fold as compared the different regimen.

In certain embodiments, a particular regimen is considered to be a de-escalation of a different regimen if the particular regimen comprises administering a smaller number of doses of a therapeutic agent than the different regimen. In certain embodiments, the particular regimen is considered to be a de-escalation as compared to the different regimen if the number of doses is decreased by about one dose, two doses, three doses, four doses, five doses, six doses, seven doses, eight doses, nine doses, ten doses, or at least ten doses, at least 15 doses, at least 20 doses, at least 25 doses, at least 30 doses, at least 35 doses, at least 40 doses, at least 45 doses, or at least 50 doses. In some embodiments, the particular regimen is considered to be a de-escalation of the different regimen if the particular regimen comprises administering at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least about 99% less doses than the different regimen.

In certain embodiments, a particular regimen is considered to be an escalation of a different regimen if the doses are administered more frequently in the particular regimen than in the different regimen. For example, in some embodiments, a particular regimen is considered to be an escalation of a different regimen if the same number of doses are administered as in the different regimen but over a shorter time frame, e.g., a regimen with three doses of an agent administered once a week over three weeks is an escalation as compared to a regimen with three doses of the agent administered once a month over three months. In certain embodiments, the particular regimen is considered to be an escalation of the different regimen if the doses in the particular regimen are administered at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, or at least about 1-fold, about 1.5 fold, about 2.0-fold, about 2.5-fold, about 3.0-fold, about 3.5-fold, about 4.0-fold, about 4.5-fold, about 5.0-fold, about 6-fold, about 7-fold, about 8-fold, about 9-fold, about 10-fold, about 15-fold, about 20-fold, about 30-fold, about 40-fold, or at least 50-fold more frequent than the doses are administered in the different regimen.

In some embodiments, a particular regimen is considered to be a de-escalation of a different regimen if the doses are administered less frequently in the particular regimen than in the different regimen. In certain embodiments, the particular regimen is considered to be a de-escalation as compared to the different regimen if the time between doses in the particular regimen is increased by at least about 1%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 110%, about 120%, about 130%, about 140%, about 150%, about 160%, about 170%, about 180%, about 190%, about 200%, or at least about 1-fold, about 1.5 fold, about 2.0-fold, about 2.5-fold, about 3.0-fold, about 3.5-fold, about 4.0-fold, about 4.5-fold, about 5.0-fold, about 6-fold, about 7-fold, about 8-fold, about 9-fold, about 10-fold, about 15-fold, about 20-fold, about 30-fold, about 40-fold, or at least 50-fold than the time between doses in the different regimen.

In certain embodiments, a particular regimen is considered to be a de-escalation of a different regimen if the particular regimen administers the same amount of the therapeutic agent as the different regimen in a split dose. In some embodiments, the split dose administered over more than one day. In certain embodiments, the particular regimen comprises administering the dose to a subject over 2 days or over 3 days. Exemplary methods for split dosing include administering 25% of the dose on the first day and administering the remaining 75% of the dose on the second day. In some embodiments, 33% of the dose may be administered on the first day and the remaining 67% administered on the second day. In some aspects, 10% of the dose is administered on the first day, 30% of the dose is administered on the second day, and 60% of the dose is administered on the third day.

In some embodiments, a particular regimen is considered to be an escalation as compared to a different regimen if the particular regimen comprises administering one or more additional therapeutic agents than the different regimen. In certain embodiments, a particular regimen is considered to be a de-escalation as compared to a different regimen if the particular regimen comprises administering one or more less therapeutic agents than the different regimen.

In some embodiments, the regimens differ in the amount of therapeutic agents that are administered. For example, in some embodiments, a first regimen may comprise administering one therapeutic agent, and a second regimen may comprise administering the first therapeutic agent with an additional therapeutic agent. For example, in certain embodiments, the first therapeutic agent may be an adoptive cell therapy, e.g., an administration of cells expressing a recombinant receptor such as a chimeric antigen receptor (CAR) and the second therapeutic agent may be a small molecule drug. As will be described, a dosing regimen is deescalated in some embodiments when a regimen produces safety concerns, including, for example, when toxicity is determined to be too high likely to be too high at a time point in an ongoing clinical trial. As will also be described, a dosing regimen is escalated in some embodiments when a regimen produces efficacy concerns, for example, when efficacy is determined to be futile or likely to be futile at a point in an ongoing clinical trial.

In some embodiments, the method includes determining a probability of a safety event and/or a response event based on information gathered from subjects previously treated in the clinical trial. In some embodiments, the methods include determining the probability of an event using a log-odds model. In some embodiments, the log-odds model is parameterized so that the initial dose level is the referent. In some embodiments, the log-odds model is parameterized such that de-escalation between regimens is constrained to be negative. In some embodiments, the log-odds model is parameterized such that escalation between regimens is constrained to be positive.

In some embodiments, the model is designed so that the joint posterior distributions of all model parameters within the regimen-toxicity model and or the regimen response model are fit and updated after information is obtained on each subject using Markov chain Monte Carlo. In some embodiments, estimates of the means and credible intervals are calculated. In some embodiments, the credible interval is or is about 99.7%, 99%, 95%, 90%, or 68%. In certain embodiments, the credible interval is 95%.

A. Regimen Toxicity Model

In some embodiments, determining a toxicity rate is based on the toxicity information of subjects previously treated across all disease cohorts for each treatment regimen. In some embodiments, a safety profile for a therapeutic agent is expected to be the same across disease subtypes. In some embodiments, a regimen-toxicity model is employed that pools safety data across disease cohorts to estimate a single probability of a toxicity event (e.g. DLT, also called a DLT rate) for each regimen across disease cohorts.

In some embodiments the safety profile for a therapeutic agent is expected to be different across disease subtypes. In some embodiments, a regimen-toxicity model is employed that does not pool safety data across disease cohorts to estimate a single probability of a toxicity event (e.g. DLT, also called a DLT rate) for each regimen across disease cohorts. In some embodiments, the regimen-toxicity model borrows data across disease cohorts to estimate a single probability of a toxicity event (e.g. DLT, also called a DLT rate) for each regimen across disease cohorts.

In particular embodiments, the toxicity rate of a regimen is determined as the clinical trial is performed. In certain embodiments, the toxicity rate is based on the toxicity information of subjects previously treated in the clinical trial across all disease cohorts for each treatment regimen. In certain embodiments, subjects in the clinical study are monitored for toxicity, e.g., a dose limiting toxicity (DLT), for a period of time to determine if there is a toxicity event associated with the treatment regimen the subject is assigned to, e.g., a DLT period. In some embodiments, the period of time, e.g., the DLT period, begins when the first dose of the treatment regimen is administered. In particular embodiments, the period of time, e.g., the DLT period, is counted from the final dose of the treatment regimen. In some embodiments, subjects are monitored for a toxicity event, e.g., a DLT, for about 1 day, about 2 days, about 3 days, about 4 days, about 5 days, about 6 days, about 7 days, about 10 days, about 14 days, about 21 days, about 28 days, about 35 days, about 42 days, about 49 days, about 56 days, about 63 days, about 70 days, about 77 days, or about 84 days after the first dose of the regimen is administered. In particular embodiments, subjects are monitored after the first dose of the treatment is administered, and are continuously monitored until about 1 day, about 2 days, about 3 days, about 4 days, about 5 days, about 6 days, about 7 days, about 10 days, about 14 days, about 21 days, about 28 days, about 35 days, about 42 days, about 49 days, about 56 days, about 63 days, about 70 days, about 77 days, or about 84 days after the last dose of the regimen is administered.

In some embodiments, posterior summaries can be calculated by tabulating the proportion of samples from the posterior distribution of the DLT rate that are less than a tolerability limit or an unacceptable limit. In some embodiments, the tolerability limit is the projected DLT rate at which a regimen is still considered safe. In some embodiments, the unacceptable limit is the projected DLT rate above which a regimen is considered unsafe. Such limits can be any rate that is determined to be appropriate. Typically, such rates are determined as part of the design of a clinical trial in consultation with physicians, which is within the skill of an ordinary person in the art.

In some embodiments, a regimen is considered safe if the dose-limiting toxicity (DLT) of the regimen is, or is expected to be, less than 50%, 45%, 40%, 35%, 34%, 33%, 31%, 30%, 29%, 28%, 27%, 26%, 25%, 24%, 23%, 22%, 21%, 20%, 15%, 10%, 5%, or 1%. In certain embodiments, the regimen may be considered unsafe if the DLT is greater than 10%, 15%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, or 95%. For example, a regimen might be considered safe if the DLT rate is less than 20% (e.g., less than 20% of subjects in a regimen experience or are expected to experience DLT), and might be considered unsafe if the DLT rate is greater than 33%. Other types of safety events can also be used, such as levels of a biomarker or qualitative scoring.

In some embodiments, such limits are defined as a probability of an expected DLT rate. In particular embodiments, a regimen is considered unlikely to be safe if there is less than a 50%, 45%, 40%, 35%, 34%, 33%, 31%, 30%, 29%, 28%, 27%, 26%, 25%, 24%, 23%, 22%, 21%, 20%, 15%, 10%, 5%, or 1% probability of a low DLT rate, e.g., a DLT rate of less than 20% or a DLT rate of less than 50%, 45%, 40%, 35%, 34%, 33%, 31%, 30%, 29%, 28%, 27%, 26%, 25%, 24%, 23%, 22%, 21%, 20%, 15%, 10%, 5%, or 1%. In certain embodiments, a regimen is considered unlikely to be safe if there is greater than a 30%, 31%, 32%, 33%, 34%, 35%, 40%, 45%, 50%, 55%, 60%, 65%, 70%, 75%, 80%, 85%, 90%, or 95% probability of an high DLT rate, e.g., a DLT rate greater than 20% or a DLT rate greater than 1%, 5%, 10%, 15%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, or 95%.

For example, in some embodiments a regimen might be considered unlikely to be safe if there is less than a 30% probability the DLT rate is less than 20%, or if there is more than a 70% probability that the DLT rate is more than 20%. As another example, in some embodiments a regimen might be considered likely to be safe if there if there is more than a 50% probability that the DLT rate is less than 33%, or if there is less than a 50% probability that the DLT rate is more than 33%.

In particular embodiments, the toxicity rate, e.g., the DLT rate, is determined by a regimen-toxicity model that estimates a single toxicity rate for each regimen across all disease cohorts. In some embodiments, safety data is pooled across disease cohorts that are included in the clinical trial. In some embodiments, a single DLT rate is estimated for each regimen across disease cohorts. In certain embodiments, the toxicity rate is determined by a formula, whereby the formula is a logit function, wherein a first regimen is set as the refrent, and wherein the logit function comprises the sum of the additive effects of the first regimen and of each additional regimen as compared to the first regimen. In particular embodiments, additive effect of a regimen is constrained to be negative if the regimen comprises administering less of a total amount of the therapeutic agent than the first regimen. In some embodiments, the additive effect of a regimen is constrained to be positive if the regimen comprises administering more of a total amount of the therapeutic agent than the first regimen.

In particular embodiments, the toxicity rate is determined by a formula, whereby the formula is a logit function and/or a log odds function. In certain embodiments, a first regimen is set as the referent, and the function comprises the sum of the additive effects of each additional regimen to the first regimen. In certain embodiments, the regimen-toxicity model comprises the formula:

$$log ( \frac{π}{1 - π} ) = {β}_{0} + {β}_{1} {X}_{1} … + {β}_{n} {X}_{n}$$

wherein π is the toxicity rate, e.g., the DLT rate, wherein β₀ is a first regimen that is set as the referent, wherein β₁ X₁ . . . βₙXₙ are the additive effects of the remaining regimens in relation to the first regimen.

For example, in some embodiments where a clinical trial is designed to have two dose levels, e.g., (d=−1,1) and two schedules e.g., (s=1,2), the DLT rate (π_{d,s}) is modeled on the log odds scale as

$$log ( \frac{{π}_{d , s}}{1 - {π}_{d , s}} ) = {β}_{0} + {β}_{1} {1}_{[ d = 1 ]} + {β}_{2} {1}_{[ s = 2 ⋂ d = 1 ]} + {β}_{3} {1}_{[ s = 2 ⋂ d = - 1 ]} .$$

In this equation, β1 is an additive effect due to dose level. In some embodiments, the model is parameterized so that the initial dose, e.g., Dose level 1, is the referent. In this example β1 is constrained to be negative, reflecting a decrease in DLT rates going from Dose level 1 to Dose level −1. In particular embodiments, β2 is an additive effect due to schedule for Dose level 1 and β3 is an additive effect due to schedule for Dose level −1. In certain embodiments, the model is parameterized so that a single-dose schedule is the referent and so β2 and β3 are constrained to be positive, reflecting an increase in the DLT rate going from a single- to a 2-dose schedule. Thus, in some embodiments, β0, the intercept term captures the log-odds of DLT rate for Dose level 1, single-dose schedule of administration. In particular embodiments, the following prior distributions are placed on each of the terms:

β₀˜N(−2.2,1.5²);β₁˜N_{(-∞,0]}(−2,1²);β₂˜N_{[0,∞)}(−10,2²);β₂N_{[0,∞)}(−10,2²).

In certain embodiments, the joint posterior distributions of all model parameters within the regimen-toxicity model are fit and updated after DLT information is obtained on each subject using Markov chain Monte Carlo. In particular embodiments, samples from the posterior distributions of the regimen-specific DLT rates, π_{d,s}, are calculated. Posterior means and 95% credible intervals are provided as estimates of each. In particular embodiments, posterior summaries are provided that include:

Probability DLT Rate is less than 33%: Pr(π_{d,s}<0.33);

Probability DLT Rate is less than 20%: Pr(π_{d,s}<0.20).

In particular embodiments, these summaries are calculated by tabulating the proportion of samples from the posterior distribution of n_{d,s} that are less than 33% or 20%.

B. Regimen Response Model

In some embodiments, determining an efficacy rate based on the response information (response outcome) of subjects previously treated within each disease cohort for each treatment regimen. In some embodiments, subjects are expected to respond to a therapeutic agent in a disease-specific or sub-type specific manner. In some embodiments, subjects across diseases or subtypes are expected to respond to a therapeutic agent the same way. In some embodiments, a regimen-response model is employed that does not pool efficacy data across disease cohorts to estimate a single probability of a response event (e.g. CR, also called a CR rate) for each regimen across disease cohorts. In some embodiments, the regimen-response model borrows data across disease cohorts to estimate a single probability of a response event (e.g. CR, also called a CR rate) for each regimen across disease cohorts.

In particular embodiments, the efficacy rate of a regimen is determined as the clinical trial is performed. In certain embodiments, the efficacy rate of a regimen is based on the response information of subjects previously treated within the disease cohort in the clinical trial. In certain embodiments, subjects in the clinical study are monitored for a response event, e.g., a CR, for a period of time to determine if there is a toxicity event associated with the treatment regimen the subject is assigned to. In some embodiments, the period of time begins when the first dose of the treatment regimen is administered. In particular embodiments, the period is counted from the final dose of the treatment regimen. In some embodiments, subjects are monitored for a response event, e.g., a CR, for about 1 day, about 2 days, about 3 days, about 4 days, about 5 days, about 6 days, about 7 days, about 10 days, about 14 days, about 21 days, about 28 days, about 35 days, about 42 days, about 49 days, about 56 days, about 63 days, about 70 days, about 77 days, or about 84 days after the first dose of the regimen is administered. In some embodiments, subjects are monitored for a response event, e.g., a CR, for between about 1 day and about 7 days, between about 7 days and about 28 days, between about 28 days and about 42 days, between 42 days and about 84 days, or for a period of time greater than 84 days. In particular embodiments, subjects are monitored after the first dose of the treatment is administered, and are continuously monitored until about 1 day, about 2 days, about 3 days, about 4 days, about 5 days, about 6 days, about 7 days, about 10 days, about 14 days, about 21 days, about 28 days, about 35 days, about 42 days, about 49 days, about 56 days, about 63 days, about 70 days, about 77 days, or about 84 days after the last dose of the regimen is administered. In some embodiments, subjects are subjects are monitored after the first dose of the treatment is administered, and are continuously monitored for between about 1 day and about 7 days, between about 7 days and about 28 days, between about 28 days and about 42 days, between 42 days and about 84 days, or for a period of time greater than 84 days after the last dose of the regimen is administered.

In some embodiments, posterior summaries can be calculated by tabulating the proportion of samples from the posterior distribution of the CR rate that are less than a success limit. In some embodiments, the success limit is the projected CR rate at which a regimen is still considered effective. Such limits can be any rate that is determined to be appropriate. Typically, such rates are determined as part of the design of a clinical trial in consultation with physicians, which is within the skill of an ordinary person in the art.

In some embodiments, a regimen is considered unlikely to be effective if the CR rate is less than 80%, 75%, 70%, 67%, 66%, 65%, 60%, 55%, 50%, 45%, 40%, 35%, 34%, 33%, 30%, 25%, 20%, 15%, 10%, or 5%. In particular embodiments, the regimen is considered likely to be effective if the CR rate is greater than 10%, 15%, 20%, 25%, 30%, 35%, 40%, 45%, 50%, 55%, 60%, 65%, 70%, 75%, 80%, 85%, 90%, 95%, or 99%. For example, a regimen might be considered unlikely to be effective if the CR rate is less than 40% (e.g., less than 40% of subjects in a regimen experience or are expected to experience CR), and might be considered effective if the CR rate is greater than 70%. Other types of responses can also be used, such as levels of a biomarker or qualitative scoring.

In some embodiments, such limits are defined as a probability of an expected CR rate. In certain embodiments, a regimen is considered unlikely to be effective if there is less than a 90%, 85%, 80%, 75%, 70%, 67%, 66%, 65%, 60%, 55%, 50%, 45%, 40%, 35%, 34%, 33%, 30%, 25%, 20% probability of a high CR rate, e.g., a CR rate greater than 40%, or a CR rate greater than 10%, 15%, 20%, 25%, 30%, 35%, 40%, 45%, 50%, 55%, 60%, 65%, 70%, 75%, 80%, 85%, 90%, 95%, or 99%. In particular embodiments, a regimen is considered unlikely to be effective is there is greater than a 20%, 25%, 30%, 35%, 40%, 45%, 50%, 55%, 60%, 65%, 70%, 75%, 80%, 85%, 90%, 95%, or 99% probability that the CR rate is low, e.g., a CR rate is less than 40%, or a CR rate that is less than 100%, 95%, 90%, 85%, 80%, 75%, 70%, 67%, 66%, 65%, 60%, 55%, 50%, 45%, 40%, 35%, 34%, 33%, 30%, 25%, 20%, 15%, 10%, or 5%. For example, in some embodiments a regimen might be considered unlikely to be effective if there is less than a 30% probability the CR rate is greater than 40%, or if there is more than a 70% probability that the CR rate is less than 40%. As another example, in some embodiments a regimen might be considered likely to be effective if there is more than a 90% probability that the CR rate is greater than 20%, or if there is less than a 10% probability that the CR rate is less than 20%.

In some embodiments, the efficacy data is not pooled across disease cohorts. In certain embodiments, the efficacy information is borrowed across disease cohorts and different CR rates are estimated across dose regimens within the different disease cohorts.

In particular embodiments, the efficacy rate, e.g., the CR rate, is determined by a regimen-efficacy model that estimates a single efficacy rate for each regimen for each disease cohort. In certain embodiments, the efficacy rate is determined by a formula, whereby the formula is a logit function, wherein a first regimen is set as the referent, and wherein the logit function comprises the sum of the additive effects of the first regimen and of each additional regimen as compared to the first regimen. In particular embodiments, additive effect of a regimen is constrained to be negative if the regimen comprises administering less of a total amount of the therapeutic agent than the first regimen. In some embodiments, the additive effect of a regimen is constrained to be positive if the regimen comprises administering more of a total amount of the therapeutic agent than the first regimen.

In some embodiments, the efficacy rate is determined by a formula, whereby the formula is a logit function and/or a log odds function. In certain embodiments, a first regimen is set as the referent, and the function comprises the sum of the additive effects of each additional regimen to the first regimen. In some embodiments, additive effect of a regimen is constrained to be negative if the regimen comprises administering less of a total amount of the therapeutic agent than the first regimen. In particular embodiments, the additive effect of a regimen is constrained to be positive if the regimen comprises administering more of a total amount of the therapeutic agent than the first regimen. In some embodiments, the regimen-efficacy model comprises the formula:

$$log ( \frac{θ}{1 - θ} ) = {α}_{0} + {α}_{1} {X}_{1} … + {α}_{n} {X}_{n}$$

wherein θ is the CR rate, wherein α₀ is a first regimen that is set as the referent, wherein α₁ X₁ . . . αₙXₙ are the additive effects of the remaining regimens in relation to the first regimen.

In particular, embodiments, for each dose level, e.g., for each dose level in a clinical study with two dose levels (d=−1,1); schedule, e.g., for each schedule in a clinical study with to schedules (s=1,2); and disease cohort; e.g., in a clinical study with two disease cohorts, one with subjects with diffuse large B cell lymphoma (DLBCL) and the other with mast cell lymphoma (MCL) (h=DLBCL,MCL); the CR rate (θ_{d,s,h}) is modeled on the log odds scale as

$$log ( \frac{{θ}_{d , s , h}}{1 - {θ}_{d , s , h}} ) = {α}_{0 , h} + {α}_{1} {1}_{[ d = 1 ]} + {α}_{2} {1}_{[ s = 2 ⋂ d = 1 ]} + {α}_{3} {1}_{[ s = 2 ⋂ d = - ]} .$$

In some embodiments, the dose level referent is Dose level 1 and the schedule referent is a single-dose schedule (−1). In certain embodiments, α1, the additive effect due to dose level is constrained to be negative and α2 and α3, the additive effects due to schedule are constrained to be positive. In some embodiments, the following prior distributions are placed on each of the regimen-response terms:

α₁˜N_{(-∞,0]}(2,1.5²);α₂˜N_{[0,∞)}(−1,1.5²);α₃˜N_{[0,∞)}(−1,1.5²).

In certain embodiments, the intercept term and estimate for the referent regimen is unique within the different disease cohorts. In particular embodiments, the following hierarchical prior distribution on the parameters:

$${α}_{0 cohort 1} , {α}_{0 cohort 2} N ( θ , {σ}^{2} ) ; θ \text{∼} N ( - 0.5 , {4}^{2} ) ; \frac{1}{{σ}^{2}} \text{∼} Gamma ( 2 , 2 ) .$$

In some embodiments, the joint posterior distributions of all model parameters within the regimen-response models are fit and updated after CR information is obtained on each subject using Markov chain Monte Carlo. In some embodiments, samples from the posterior distributions of the regimen and disease cohort-specific CR rates, θ_{d,s,h}, are calculated. In particular embodiments, posterior means and 95% credible intervals are provided as estimates of each. In some embodiments, the following posterior summaries for each regimen and disease cohort are provided:

Probability CR rate is greater than 25%: Pr(θ_{d,s,h}>0.255);

Probability CR rate is greater than 40%: Pr(θ_{d,s,h}>0.40).

In some embodiments, these summaries are calculated by tabulating the proportion of samples from the posterior distribution of θ_{d,s,h} that are greater than 25% or 40%.

C. Selection of Priors

In particular embodiments, prior values and distributions are chosen. In particular embodiments, under the regimen toxicity model and regimen-response model, mean DLT and CR rates and credible intervals are given for each of the dose regimen. In some embodiments, the credible intervals are 99.9%, 99%, 98%, 97.5%, 97%, 95%, 90%, 85%, or 80% credible intervals. In particular embodiments, the credible intervals are 95% credible intervals.

In some embodiments, prior values and distributions are chosen to be relatively non-informative while still reflecting expected prior beliefs of the probability of a DLT and CR for each dose regimen.

In some embodiments, a prior value and distribution for the DLT rate of a regimen is provided. In particular embodiments, the value is a mean or mode DLT rate of about 1%, about 5%, about 10%, about 15%, about 20%, about 25%, about 30%, about 35%, about 40%, about 45%, about 50%, about 55%, or about 60%, or the value is a mean or mode DLT rate that falls between 0% and 10%, between 10% and 20%, between 20% and 30%, between 30% and 40%, between about 40% and 50%, or between about 50% and 60%. In particular embodiments, the lower boundary of the credible interval, e.g., a 95% credible interval, of the mean or mode DLT rate is about 0%, about 1%, about 2%, about 3%, about 4%, about 5%, about 6%, about 7%, about 8%, about 9%, about 10%, or is a value that falls between 0% and 5%, between 5% and 10%, between about 10% and 20%, between about 20% and about 30%, between about 30% and about 40%, between about 40% and about 50%, or between about 50% and about 60%. In particular embodiments, the upper boundary of the credible interval, e.g., a 95% credible interval, of the mean or mode DLT rate is about 30%, 35%, 40%, 45%, 50%, 55%, 60%, or greater than 60%, or is a value that falls between 30% and 40%, between 40% and 50%, between 50% and 60%, or between 60% and 70%.

In some embodiments, a prior value and distribution for the CR rate of a regimen is provided. In particular embodiments, the value is a mean or mode CR rate of about 15%, about 20%, about 25%, about 30%, about 35%, about 40%, about 45%, about 50%, about 55%, about 60%, 65%, about 70%, about 75%, about 80%, about 85%, about 90%, about 95%, or the value is a mean DLT rate that falls between 15% and 30%, between 30% and 50%, between 50% and 60%, between 60% and 70%, between about 70% and 80%, between about 80% and 90%, between about 90% and 95%, or between about 95% and 100%. In particular embodiments, the lower boundary of the credible interval, e.g., a 95% credible interval, of the mean or mode CR rate is about 0%, about 1%, about 2%, about 3%, about 4%, about 5%, about 6%, about 7%, about 8%, about 9%, about 10%, or is a value that falls between 0% and 5%, between 5% and 10%, between about 10% and 20%, between about 20% and about 30%, between about 30% and about 40%, between about 40% and about 50%, or between about 50% and about 60%. In particular embodiments, the upper boundary of the credible interval, e.g., a 95% credible interval, of the mean or mode CR rate is about 40%, about 45%, about 50%, about 55%, about 60%, about 65%, about 70%, about 75%, about 80%, about 85%, about 90%, about 95%, about 98%, about 99%, or about 100%, or is a value that falls between 30% and 60%, between 60% and 70%, between about 70% and 80%, between about 80% and 90%, between about 90% and 95%, or between about 95% and 100%.

In some embodiments, the priors for the regimen-response model within each disease cohort are the same. In particular embodiments, the priors for the regimen-response model within each disease cohort are different. In certain embodiments, the priors put the highest probability (the prior mode) on the belief that all regimens are likely to be safe (e.g., DLT rate<0.33) and most are likely effective (CR rate>0.40). In some embodiments, a regimen with a posterior mode outside this range would not be suitable for the trial. In some embodiments, priors are chosen when there is little available human safety or efficacy data available. In certain embodiments, the priors are relatively non-informative and stretch into the unsafe or non-efficacious range. In some embodiments, priors are chosen to produce ethical and efficient changes in doses and regimens based upon accruing safety and efficacy data, for example, in a manner similar to how a 3+3 design produces desirable operating characteristics.

D. Utility Score

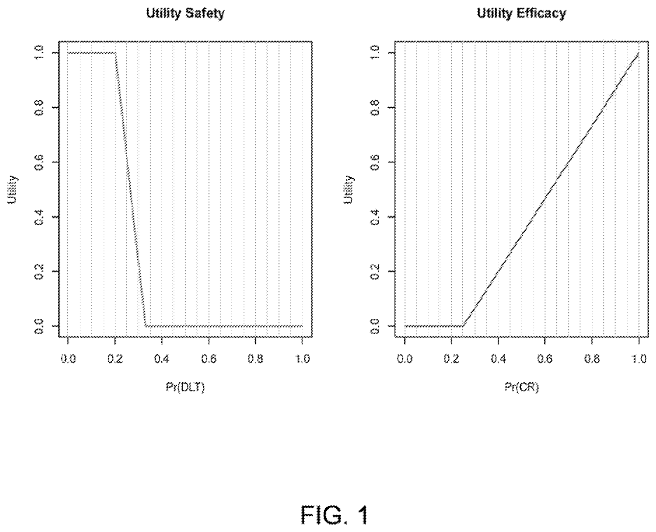

In some embodiments, the methods comprise calculating an overall disease-specific utility score for each disease subtype cohort for each treatment regimen. In some embodiments, the utility score balances toxicity with response information (e.g. antitumor activity) so fewer subjects will be allocated to regimens that appear to be unsafe or ineffective. In some embodiments, the disease specific utility is constructed based on target safety and efficacy parameters to determine a safety utility score and an efficacy utility score, respectively, for each treatment regimen. An exemplary set of utility functions are depicted in FIG. 1.

FIG. 1 (left) shows an exemplary utility function for safety. In the exemplary utility function for safety of FIG. 1, utility is defined as 1 if the DLT rate is less than or equal to 20%. Conversely, in FIG. 1, utility is defined as 0 if the DLT rate is N33%. Between a 20% and 33% DLT rate, utility decreases linearly as the DLT rate increases. Specifically, utility decreases 0.77 units for every 10% increase in the DLT rate.

FIG. 1 (right) shows an exemplary utility function for efficacy, where the utility is defined as 0 if the CR rate is less than 25%. In this example, beyond a 25% CR rate in the exemplary utility function, the utility increases as the CR rate increases linearly, at the rate of a 0.13-unit increase in utility for every 10% increase in the CR rate.

In some embodiments, the safety utility score is defined as 1 when the probability of the toxicity event is projected to be less than or equal to a tolerability limit. In some embodiments, the safety utility score is defined as 1 when the probability of the toxicity event is projected to be less than or equal to a DLT rate of about 1%, about 5%, about 10%, about 15%, about 20%, about 25%, about 30%, about 35%, about 40%, about 45%, about 50%, about 55%, or about 60%, or a DLT rate that falls between 0% and 10%, between 10% and 20%, between 20% and 30%, between 30% and 40%, between about 40% and 50%, or between about 50% and 60%. In some embodiments, the safety utility score is defined as 0 the probability of the toxicity event is projected to be greater or equal to an unacceptable limit. In certain embodiments, the safety utility score is defined as 0 when the probability of the toxicity event is projected to be greater than or equal to a DLT rate of about 1%, about 5%, about 10%, about 15%, about 20%, about 25%, about 30%, about 35%, about 40%, about 45%, about 50%, about 55%, or about 60%, or a DLT rate that falls between 0% and 10%, between 10% and 20%, between 20% and 30%, between 30% and 40%, between about 40% and 50%, or between about 50% and 60%. In some embodiments, the utility score decreases linearly as the probability of the toxicity event increases.

In some embodiments, the efficacy utility score is defined as 0 if the probability of the efficacy event is projected to be less than or equal to a success limit. In particular embodiments, the efficacy utility score is defined as 0 if the probability of the efficacy event is projected to be less than or equal to a CR rate of about 10%, about 15%, about 20%, about 25%, about 30%, about 35%, about 40%, about 45%, about 50%, about 55%, or about 60%, or a DLT rate that falls between 0% and 10%, between 10% and 20%, between 20% and 30%, between 30% and 40%, between about 40% and 50%, or between about 50% and 60%. In some embodiments, the efficacy utility score increases linearly as the probability of the efficacy event increases. In certain embodiments, the efficacy score is 1 when the efficacy event is or is projected to be 100%.

In some embodiments, generating a safety utility score is a function of the probability of the toxicity event for the treatment regimen. In some embodiments, generating an efficacy utility score is a function of the probability of the efficacy event for the treatment regimen.

In some embodiments, the overall disease-specific utility score is based on response information of subjects within each disease subtype cohort previously treated with the therapeutic agent according to each treatment regimen and on toxicity information of subjects across all disease cohorts previously treated with the therapeutic agent according to each treatment regimen. In some embodiments, calculating the overall disease-specific utility score for each disease subtype cohort for each treatment regimen comprises multiplying the safety utility score by the efficacy utility score.

E. Adaptive Allocation to Open and Eligible Regimens

Generally, a subject enrolling in a clinical trial can only be allocated to an open regimen. In some embodiments, the trial begins by opening a single regimen, while the remaining regimens remain closed. In some embodiments, one or more of the remaining regimens comprise escalations relative to the initial regimen. In some embodiments, one or more of the remaining regimens comprise de-escalations relative to the initial regimen. In some embodiments, each of the unique regimens can be an escalation relative to another regimen and/or a de-escalation relative to another regimen. Escalations and de-escalations can comprise differences in dose, schedule, and/or number of doses.

In particular embodiments, a regimen must be open for enrollment for subjects to be assigned to that regimen. In certain embodiments, a regimen must not be suspended for enrollment for subjects to be assigned to that regimen. In some embodiments, a regimen must be both open and not suspended for enrollment for subjects to be assigned to that regimen. In certain embodiments, if a single regimen is open and not suspended, all subjects are assigned are assigned to that regimen. In some embodiments, if more than one regimen is open and not suspended for enrollment, subjects are adaptively randomized to all the regimens that are open and not suspended for enrollment. In particular embodiments, there are many methods that will be recognized by one of skill in the art that are suitable for determining that adaptive randomization assignment probability for each regimen.

In some embodiments, allocation to open and eligible regimens is based on the disease cohort-specific utility for each regimen as calculated as described under the “Utility Score” section above, the relative uncertainty of the estimate for the utility of each regimen, and the number of subjects already allocated to the regimen within the disease cohort.

In certain embodiments, the randomization probability of enrollment to each regimen that is open and/or eligible is:

$${V}_{x} ∝ {[ \frac{Pr ( {r}_{x} = {r}_{{x}^{*}} ) Var ( {U}_{x} )}{{n}_{x} + 1} ]}^{\frac{1}{2}} .$$

wherein rₓ is the regimen x, Pr(rₓ=r_{x*}) is the probability the regimen x is the highest utility regimen, Var(Uₓ) is the variance of the regimen's utility score, and nₓ is the number of subjects already allocated to the regimen.

For example, in some embodiments, the randomization probability of enrollment to each regimen within each disease cohort, V_{d,s,h}, is:

$${V}_{d , s , h} ∝ {[ \frac{Pr ( {r}_{d , s , h} = {r}_{{d}^{*} , {s}^{*} , h} ) Var ( {U}_{d , s , h} )}{{n}_{d , s , h} + 1} ]}^{\frac{1}{2}} .$$

where r_{d,s,h} represents the regimen of dose level d, schedules, and disease h, and Pr(r_{d,s,h}=r_{d*,s*,h}) is the probability the regimen is the highest utility regimen, Var(U_{d,s,h}) is the variance of the regimen's estimated disease-specific utility score, and n_{d,s,h} are the number of subjects already allocated to the regimen within disease cohort h.

In some embodiments, this method is statistically efficient. For example, in a 2-arm trial, maximum efficiency can come from 1:1 randomization. Thus, in some embodiments, if two regimens are performing similarly in terms of utility, the regimen with the smaller sample size and/or higher variance can be favored for enrollment. In certain embodiments, increasing the randomization probability to the regimen with the smaller sample size can steer the trial back towards equal randomization between the best regimens and maximum statistical efficiency.

Generally, subjects are allocated by adaptive randomization to all open and eligible regimens. In some embodiments, when only one regimen is open, all subjects are assigned to the one open regimen. In some embodiments, when only one regimen is open and eligible for enrollment, all patents are assigned to the one open and eligible regimen.

In some embodiments, subjects are adaptively randomized to all regimens that are open and eligible for enrollment if more than one regimen is open.

In some embodiments, regimens with higher utility receive a greater randomization probability. In some embodiments, the method for allocating subjects balances allocating subjects to regimens to increase the certainty of utility estimates while also preferentially allocating subjects to regimens with the highest utility. In some embodiments, the method for allocating subjects is designed so that randomization probabilities will be more dispersed across the open regimens if there is a large amount of uncertainty in the estimates of the utility for each regimen. In some embodiments, the method is balanced between allocating subjects to regimens to increase the certainty of utility estimates while also preferentially allocating subjects to regimens with the highest utility such that randomization probabilities will not focus on a single regimen prematurely if there is still a large amount of uncertainty as to which regimen has the highest utility.

F. Assignment of the Regimen

In particular embodiments, there is one open regimen at the beginning of the clinical trial. In certain embodiments, there is one open regimen when subjects begin to be assigned to regimens for the clinical trial. In certain embodiments, when one, two, three, four, five, six, seven, eight, nine, ten, or more than ten subjects have completed the DLT period in the initial regimen, other regimens may be opened. In particular embodiments, when one, two, three, four, five, six, seven, eight, nine, ten, or more than ten subjects across disease cohorts have completed the DLT period on the initial regimen, other regimens may be opened. In certain embodiments, when three subjects have completed the DLT period on the initial regimen, other regimens may be opened. In some embodiments, when three subjects across disease cohorts have completed the DLT period on the initial regimen, other regimens may be opened. In some embodiments, an additional regimen may be opened based on safety or efficacy.

G. Entry into the Trial

In certain embodiments, the clinical trial has open enrollment. In some embodiments, open enrollment indicates that subjects are enrolled as they become available for the clinical trial. Particular embodiments contemplate that open enrollment has advantages over cohort enrollment, such as but not limited to that few or no pauses are required while waiting for the completion of the toxicity evaluation period, e.g., the DLT period, for all subjects in the cohort.

In some embodiments, the accrual rate into the trial is governed by allowing only a certain number of patients enrolled with unknown DLT information per regimen. For example, in some embodiments, no more than one, two, three, four, five, six, seven, eight, nine, ten, or greater than ten subjects with unknown toxicity information, e.g., DLT information, are allowed in a given regimen. In particular embodiments, no more than three with unknown toxicity information, e.g., DLT information, are allowed in a given regimen. In particular embodiments, one or more additional subjects with unknown toxicity are allowed in the given regimen when there are at least three, four, five, six, seven, eight, nine, or ten subjects with complete toxicity, e.g., DLT, information. In some embodiments, four subjects with unknown toxicity are allowed in the given regimen when there are at least three, four, five, six, seven, eight, nine, or ten subjects with complete toxicity, e.g., DLT, information. In particular embodiments, four subjects with unknown toxicity are allowed in the given regimen when there are at least six subjects with complete toxicity, e.g., DLT, information. Thus, in some embodiments, the number of subjects with unknown toxicity information in a given regimen, i.e, the queue, is determined by the number of subjects with complete toxicity, e.g., DLT, information and the number of subjects with unknown toxicity information across disease cohorts.

H. Opening a Regimen

In some embodiments, the decision to open another regimen is made because of the efficacy or response information observed from subjects in an open regimen. In some embodiments, the decision to open another regimen is made because an open regimen is considered to be safe, effective, unsafe, or ineffective. In some embodiments, a treatment regimen is determined to be unsafe for the purpose of opening another regimen if the probability that the toxicity rate is greater than a safety limit is greater than a toxicity probability parameter. In some embodiments, a treatment regimen is determined to be safe for the purpose of opening another regimen if the probability that the toxicity rate is below a safety target is greater than a safety probability parameter. In some embodiments, the safety limit, the probability of the toxicity rate, and/or the toxicity probability parameter can be the same or different for determining if a regimen is safe or unsafe. For example, in some embodiments the trial can be designed such that the probability of the toxicity rate for determining that the regimen is safe can be higher or lower than the probability of the toxicity rate for determining that the regimen is unsafe. As a non-limiting example, a regimen might be considered safe if Pr(π_{d,s}<safety limit)>toxicity probability parameter (e.g., (Pr(π_{d,s}<33%)>70%)), whereas the regimen might be considered unsafe if Pr(η_{d,s}<safety limit)<toxicity probability parameter (e.g. (Pr(π_{d,s}<33%)<50%)).

In some embodiments, a treatment regimen is determined to be ineffective for the purpose of opening another regimen if the probability that the efficacy rate is greater than an efficacy target is less than an efficacy probability parameter (for example, if Pr(θ_{d,s,h}>efficacy target)<efficacy probability parameter). In some embodiments, a treatment regimen is determined to be effective for the purpose of opening another regimen if the probability that the efficacy rate is greater than an efficacy target is greater than an efficacy probability parameter (for example, if (θ_{d,s,h}>efficacy target)>efficacy probability parameter). In some embodiments, the efficacy rate, probability of the efficacy rate, and efficacy target or efficacy limit can be the same or different for determining if a regimen is effective or ineffective.

In some embodiments, if a first regimen is open and is determined to be unsafe, a second regimen that is a de-escalation of the first regimen is opened. In some embodiments, if a first regimen is open and is determined to be ineffective, a second regimen that is an escalation of the first regimen is opened. In some embodiments, if a first regimen is open and is determined to be safe but ineffective, a second regimen that is an escalation of the first regimen is opened. In some embodiments, if a first regimen is considered to be unsafe but effective, a second regimen that is a de-escalation of the first regimen is opened. In some embodiments, if a first regimen is open and is determined to be safe, and a second regimen is opened and determined to be unsafe, a third regimen that is an escalation of the first regimen and a de-escalation of the second regimen is opened. In some embodiments, if a first regimen is open and is determined to be effective, and a second regimen is open and determined to be ineffective, a third regimen that is a de-escalation of the first regimen and an escalation of the second regimen is opened. In some embodiments, if a first regimen is opened and determined to be effective but unsafe, and a second regimen is open determined to be ineffective but safe, a third regimen that is a de-escalation of the first regimen and an escalation of the second regimen is opened.

It is to be understood that in some embodiments, “determined” can be an estimate or a projection of the relative safety or efficacy of a regimen by any of the methods described herein.

In some embodiments, a regimen is opened because of the safety information of an open regimen. In some embodiments, a regimen is opened because an open regimen is determined to be or is determined to be likely of being unsafe. In particular embodiments, a regimen is opened because the open regimen has less than 50%, 45%, 40%, 35%, 34%, 33%, 31%, 30%, 29%, 28%, 27%, 26%, 25%, 24%, 23%, 22%, 21%, 20%, 15%, 10%, 5%, or 1% probability of a low toxicity rate, e.g., a DLT rate of less than 20% or a DLT rate of less than 50%, 45%, 40%, 35%, 34%, 33%, 31%, 30%, 29%, 28%, 27%, 26%, 25%, 24%, 23%, 22%, 21%, 20%, 15%, 10%, 5%, or 1%. In certain embodiments, a regimen is opened because the open regimen has greater than a 30%, 31%, 32%, 33%, 34%, 35%, 40%, 45%, 50%, 55%, 60%, 65%, 70%, 75%, 80%, 85%, 90%, or 95% probability of an high toxicity rate, e.g., a DLT rate greater than 20% or a DLT rate greater than 1%, 5%, 10%, 15%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, or 95%. In some embodiments, a regimen is opened because an open regimen is determined to be unsafe. In some embodiments, a regimen is opened because an open regimen has less than a 50% chance of having a low toxicity rate, e.g., a DLT rate, of less than 33%.

In certain embodiments, a regimen is opened because an open regimen is determined to be or is determined to be likely of being safe. In particular embodiments, a regimen is opened because the open regimen has greater than a 30%, 33%, 34%, 35%, 40%, 45%, 50%, 55%, 60%, 65%, 70%, 75%, 80%, 85%, 90%, 95%, 97%, 98%, or greater than 99% probability of a low toxicity rate, e.g., a DLT rate of less than 33% or a DLT rate of less than 20%, 25%, 30%, 35%, 40%, 45%, 50%, 55%, 60%, 65%, 70%, 75%, 80%, 85%, 90%, 95%, 97%, 98%, or greater than 99%. In certain embodiments, a regimen is opened because the open regimen has less than a 50%, 45%, 40%, 35%, 34%, 33%, 31%, 30%, 29%, 28%, 27%, 26%, 25%, 24%, 23%, 22%, 21%, 20%, 15%, 10%, 5%, or 1% probability of an high toxicity rate, e.g., a DLT rate greater than 33% or a DLT rate greater than 20%, 25%, 30%, 33%, 34%, 35%, 40%, 45%, 50%, 55%, 60%, 65%, 70%, 75%, 80%, 85%, 90%, or 95%. In some embodiments, a regimen is opened because an open regimen is determined to be safe. In some embodiments, a regimen is opened because an open regimen has greater than a 70% chance of having a low toxicity rate, e.g., a DLT rate, of less than 33%.

In certain embodiments, a regimen is opened because of the efficacy information of an open regimen. In some embodiments, a regimen is opened because an open regimen is determined to be or is determined to be likely of being ineffective. In particular embodiments, a regimen is opened because the open regimen has less than a 50%, 45%, 40%, 35%, 34%, 33%, 31%, 30%, 29%, 28%, 27%, 26%, 25%, 24%, 23%, 22%, 21%, 20%, 15%, 10%, 5%, or 1% probability of a high efficacy rate, e.g., a CR rate of greater than 20%, a CR rate of greater than 70%, or a CR rate of greater than 20%, 25%, 30%, 35%, 40%, 45%, 50%, 55%, 60%, 65%, 70%, 75%, 80%, 85%, 90%, 95%, 97%, 98%, or 99%. In certain embodiments, a regimen is opened because the open regimen has greater than a 30%, 31%, 32%, 33%, 34%, 35%, 40%, 45%, 50%, 55%, 60%, 65%, 70%, 75%, 80%, 85%, 90%, or 95% probability of a low efficacy rate, e.g., a CR rate of less than 20%, a CR rate of less than 70%, or a CR rate greater than 70%, 65%, 60%, 55%, 50%, 45%, 40%, 35%, 30%, 25%, 20%, 15%, 10%, 5%, or 1%. In some embodiments, a regimen is opened because an open regimen is determined to be ineffective. In some a embodiments, a regimen is opened because an open regimen has less than a 30% probability that the CR rate is greater than 40%. In certain embodiments, a regimen is opened because an open regimen has greater than a 70% probability that the CR rate is less than 40%.

In some embodiments, a regimen is opened if an open regimen is or is likely to be effective. In particular embodiments, a regimen is opened because the open regimen has greater than a 30%, 33%, 34%, 35%, 40%, 45%, 50%, 55%, 60%, 65%, 70%, 75%, 80%, 85%, 90%, 95%, 97%, 98%, or greater than 99% probability of a high efficacy rate, e.g., a CR rate that is greater than 20%, 25%, 30%, 35%, 40%, 45%, 50%, 55%, 60%, 65%, 70%, 75%, 80%, 85%, 90%, 95% or 99%. In certain embodiments, a regimen is opened because the open regimen has less than a 50%, 45%, 40%, 35%, 34%, 33%, 31%, 30%, 25%, 20%, 15%, 10%, 5%, or 1% probability of a low efficacy rate, e.g., CR rate, of less than 50%, 45%, 40%, 35%, 34%, 33%, 31%, 30%, 25%, 20%, 15%, 10%, 5%, or 1%. In some embodiments, a regimen is opened when an open regimen has more than a 90% probability that the efficacy rate, e.g., the CR rate, is greater than 20%. In particular embodiments, a regimen is opened when an open regimen has less than a 10% probability that the efficacy rate, e.g., the CR rate, is less than 20%.

In certain embodiments, a regimen is opened that de-escalates from an open regimen. In some embodiments, a regimen is opened that de-escalates from the open regimen across disease cohorts. In certain embodiments, a regimen is opened that de-escalates from the open regimen based, at least in part, on toxicity information, e.g., the DLT rate, from the open regimen. In certain embodiments, a new regimen that de-escalates from the open regimen is opened when the open regimen has at least 3 completers and is unlikely to be safe across disease cohorts. In some embodiments, a regimen will be opened that de-escalates from the open regimen based on the dose of the therapeutic agent. In certain embodiments, a regimen will be opened that de-escalates from the open regimen based on the schedule of the administration therapeutic agent.

In particular embodiments, a regimen is opened that escalates from an open regimen. In some embodiments, a regimen is opened that escalates from the open regimen within a disease cohort. In certain embodiments, a regimen is opened that escalates from the open regimen based, at least in part, on toxicity information, e.g., the DLT rate, from the initial regimen. In certain embodiments, a new regimen that escalates from the open regimen is opened when the initial regimen has at least 12 subjects enrolled and is likely to be safe across disease cohorts. In some embodiments, a regimen will be opened that escalates from the open regimen based on the dose of the therapeutic agent. In certain embodiments, a regimen will be opened that escalates from the open regimen based on the schedule of the administration therapeutic agent.

In some embodiments, a regimen is opened that escalates from an open regimen based on efficacy information, e.g., CR rate. In some embodiments, a regimen is opened that escalates from the open regimen within a disease cohort. In certain embodiments, a regimen is opened that escalates from the open regimen based, at least in part, on efficacy information, e.g., CR rate, from the open regimen. In certain embodiments, a new regimen that escalates from the open regimen is opened when the open regimen has at least 3 completers and is unlikely to be effective within a disease cohort. In some embodiments, a regimen will be opened that escalates from the open regimen based on the dose of the therapeutic agent. In certain embodiments, a regimen will be opened that de-escalates from the open regimen based on the schedule of the administration therapeutic agent.

I. Methods for Suspending or Resuming Enrollment of Subjects to a Regimen

In some embodiments, an open regimen can have a second status that can determine whether additional subjects can be enrolled in the regimen. In some embodiments, the second status can be “eligible” or “suspended.” In some embodiments, the second status of a regimen is set to eligible when the regimen opens. In some embodiments, second status of a regimen is changed because the regimen is determined to be safe, effective, unsafe, or ineffective. The status of a regimen can be temporary, and can change as additional safety and efficacy outcomes are determined during the course of a clinical trial as more subjects complete observation periods.

In some embodiments, the second status is changed to suspended for safety, e.g. if the regimen is determined to be unsafe. In some embodiments, the second status is changed to suspended if the probability that the toxicity rate is less than a safety limit is less than a toxicity probability parameter. For example, if Pr(π_{d,s}<safety limit)<safety parameter (e.g. if Pr(π_{d,s}<20%)<30%).

In some embodiments, the second status is changed to suspended for futility, e.g. if the regimen is determined to be ineffective. In some embodiments, the second status is changed to suspended if the probability that the efficacy rate is greater than an efficacy target is less than an efficacy probability parameter. In some embodiments, the second status is changed to suspended if Pr(θ_{d,s,h}>efficacy target)<efficacy probability parameter (e.g. if Pr(θ_{d,s,h}>40%)<30%).

In some embodiments, the second status is changed to suspended for success. In some embodiments, the second status is changed to suspended if the regimen is determined to be both safe and effective. In some embodiments, a regimen is determined to be both safe and effective if the probability that the toxicity rate is less than a safety limit is greater than a toxicity probability parameter and if the probability that the efficacy rate is greater than an efficacy target is greater than an efficacy probability parameter. For example, Pr(n_{d,s}<safety limit)>toxicity probability parameter and (θ_{d,s,h}>efficacy target)>efficacy probability parameter. In some embodiments, any or all of the safety targets, safety probability parameters, and safety limits can be the same or different for suspending a regimen for safety or success or for opening a regimen for safety or success. Likewise, in some embodiments, any or all of the efficacy targets and efficacy probability parameters can be the same or different for suspending a regimen for efficacy or futility or for opening a regimen for efficacy or futility. It is within the capabilities of a person of ordinary skill in the art to determine the appropriate parameters for opening, suspending or resuming enrollment for a regimen.

In some embodiments, subjects may only be allocated to a regimen if is likely to be safe across disease cohorts at all times during the clinical trial. In certain embodiments, enrollment to a regimen will be suspended for safety across all cohorts, e.g., disease cohorts, if the toxicity rate, e.g., DLT rate, is determined to be or is likely to be greater than 20%, 25%, 30%, 31%, 32%, 33%, 34%, 35%, 40%, 45%, 50%, 55%, 60%, 65%, 70%, 75%, 80%, 85%, 90%, or 95%, and has a probability of greater than 40%, 45%, 50%, 55%, 60%, 65%, 70%, 75%, 80%, 85%, 90%, or 95% probability that the toxicity rate, e.g., DLT rate, is greater than 20%, 25%, 30%, 31%, 32%, 33%, 34%, 35%, 40%, 45%, 50%, 55%, 60%, 65%, 70%, 75%, 80%, 85%, 90%, or 95%. In certain embodiments, enrollment to a regimen will be suspended for safety across all cohorts, e.g., disease cohorts, if the toxicity rate, e.g., DLT rate, is determined to be or is likely to be greater than 33%, and has a probability of greater than 50% probability that the toxicity rate, e.g., DLT rate, is greater than 33%.

In certain embodiments, enrollment to a regimen may be temporarily suspended for futility within a disease cohort. In particular embodiments, enrollment to regimens other than the highest utility regimen may be temporarily suspended for early success within disease cohorts. In some embodiments, if a regimen has one, two, three, four, five, six, seven, eight, nine, ten, or more than ten completers and has an observed utility score of less than 40%, 35%, 30%, 25%, 20%, 15%, 10%, 5%, or 1% within a disease cohort, enrollment to that regimen will be suspended for futility within that cohort. In particular embodiments, if a regimen has six completers and has an observed utility score of less than 5% within a disease cohort, enrollment to that regimen will be suspended for futility within that disease cohort.

In certain embodiments, a regimen may be suspended for early success. In particular embodiments, the regimen may be suspended for early success if the regimen is not the highest utility regimen. In particular embodiments, the regimen may be suspended for early success if the regimen has six, seven, eight, nine, ten, eleven, twelve, thirteen, fourteen, fifteen or more completers within a disease cohort. In certain embodiments, the regimen may be suspended for early success if the regimen has twelve completers within a disease cohort. In some embodiments, the regimen may be suspended for early success if the regimen is highly likely to be both safe and effective. In particular embodiments, the regimen may be suspended for early success if the regimen is not the highest utility regimen, has twelve completers within a disease cohort, and is highly likely to be both safe and effective. In some embodiments, the regimen may be suspended for early success if the regimen has a greater than 50%, 55%, 60%, 65%, 70%, 75%, 80%, 85%, 90%, 95%, or greater than a 99% probability of having a low toxicity, e.g., a DLT rate of less than 40%, 355, 34%, 33%, 30%, 25%, 20%, 15%, 10%, 5%, or 1%, and a greater than 75%, 80%, 85%, 90%, 95%, or greater than 99% chance of having a efficacy rate, e.g., a CR rate, of greater than 20%, 25%, 30%, 31%, 32%, 33%, 34%, 35%, 40%, 45%, or 50%. In certain embodiments, the regimen may be suspended for early success if the regimen has a greater than 70% probability of having a low toxicity, e.g., a DLT rate of less than 33%, and a greater than a 90% chance of having a efficacy rate, e.g., a CR rate, of greater than 25%. In some embodiments, a regimen can resume enrollment, or the suspension can be lifted, if additional subject outcomes for a particular regimen or cohort include subject outcomes with complete responses or lacking toxicity. In some embodiments, this includes subject who have received the therapy defined for a particular regimen, but who have not yet completed the observation period when the regimen is suspended. In some embodiments, the suspension can be lifted as those subjects complete their observation periods and if their outcomes are sufficient to change the status of the regimen.

It is to be understood that in some embodiments, “determined” can be an estimate or a projection of the relative safety or efficacy of a regimen by any of the methods described herein.

J. Methods for Stopping the Trial Early

In some embodiments, the trial can be stopped early. Exemplary criteria for stopping a trial early include success, toxicity, or futility. The criteria for stopping a trial early can be the same or different than the criteria for suspending a regimen within or across disease cohorts.

In some embodiments, the clinical trial protocol can include a maximum number of total subjects that can be enrolled in the trial. In some embodiments, the clinical trial can end when the total number of subjects meets or exceeds the maximum number of total subjects. Likewise, the clinical trial can include a maximum number of subjects with a particular disease or sub-disease that can be enrolled. In some embodiments, the clinical trial can end when the total number of subjects with each disease or sub-disease meets or exceeds the maximum number of subjects allowed for each disease or sub-disease.

In particular embodiment, the clinical trial will be continuously monitored and may be stopped early for either futility or success. In some embodiments, early stopping based on efficacy data will be disease cohort-specific. In some embodiments, stopping rules are determined based on the treatment effect of existing therapies for this trial patient population, and the expected efficacy of the therapeutic agent, e.g., a CAR T cell therapy. In some embodiments, if one disease cohort meets the enrollment stopping criteria and the other cohort does not, the trial will continue enrolling patients only from the other disease cohort until the enrollment stopping criteria is met. In certain embodiments, the trial will continue as long as at least one disease cohort is enrolling or until the maximum total sample size of subjects across the disease cohorts is reached. In some embodiments, a disease cohort may be stopped if the maximum total sample size of subjects within the disease cohort is reached.

In some embodiments, the maximum total sample size of subject across the disease cohorts is a number of subjects between 20 and 100 subjects, between 30 and 40 subjects, between 40 and 50 subjects, between 45 and 75 subjects, between about 55 and 65 subjects, between about 60 and 80 subjects, between about 70 and 90 subjects, between about 80 and 100 subjects. In particular embodiments, the maximum number of subjects across the disease cohorts is 60 subjects.

In certain embodiments, enrollment of subjects in a disease cohort may be stopped if the maximum total sample size of subjects within the disease cohort is reached. In certain embodiments, the maximum total sample size of subject within a disease cohorts is a number of subjects between 10 and 50 subjects, between 10 and 20 subjects, between 20 and 30 subjects, between 30 and 40 subjects, between about 40 and 50 subjects. In particular embodiments, the maximum total sample size of subjects within the disease cohort is 35 subjects.

In some embodiments, subjects can only be enrolled in open regimens that are eligible for enrollment (for example, regimens that are not suspended). In some embodiments, the clinical trial can end when there are no open and eligible regimens. In some embodiments, this can occur when all of the regimens are either closed or suspended.

In some embodiments, the trial can end when response and/or toxicity information within each open disease cohort has been determined for a first pre-determined minimum number of subjects within each open regimen, the regimen with the highest utility is unlikely to be effective and/or safe within the cohort for the purpose of termination, and no more regimens can be opened. In some embodiments, a regimen is unlikely to be effective within the cohort for the purpose of termination if the probability that the efficacy rate is greater than a termination efficacy target is less than a termination efficacy probability parameter. For example, in some embodiments, a regimen is unlikely to be effective within the cohort for the purpose of termination Pr(θ_{d,s,h}>termination efficacy target)<termination efficacy probability parameter. The termination efficacy target and/or termination efficacy probability parameter can be the same or different than the efficacy targets and/or efficacy probability parameters used for opening or suspending a regimen.

In some embodiments, a regimen is unlikely to be safe within or across cohorts for the purpose of termination if the probability that the toxicity rate is less than a termination safety limit is less than a termination toxicity probability parameter. For example, in some embodiments, a regimen is unlikely to be safe within or across cohorts for the purpose of termination Pr(π_{d,s}<termination safety limit)<termination toxicity probability parameter.

In some embodiments, the trial can end when response and/or toxicity information within each open disease cohort has been determined for a second pre-determined minimum number of subjects within each open regimen, the regimen with the highest utility is likely to be effective and/or safe within the cohort for the purpose of termination, and/or no more regimens can be opened. In some embodiments, a regimen is likely to be effective for the purpose of termination if the probability that the efficacy rate is greater than an early success efficacy target is greater than an early success efficacy probability parameter. In some embodiments, a regimen is likely to be safe for the purpose of termination if the probability that the toxicity rate is less than an early success safety limit is greater than an early success toxicity probability parameter. For example, in some embodiments a regimen is likely to be safe and effective for the purpose of termination if Pr(n_{d,s}<early success safety limit)>early success toxicity probability parameter and (θ_{d,s,h}>early success efficacy target)>early success efficacy probability parameter (e.g. if (Pr(π_{d*,s*}<33%)>70% and Pr(θ_{d*,s*,h}>25%)>90%).

K. Method for Determining if the Clinical Trial is a Success

In some embodiments, the clinical trial will be deemed a success the trial terminates for early success or if the regimen with the highest utility score for a particular disease cohort at the conclusion of the trial has a probability that the toxicity rate is less than a final success safety limit that is greater than a final success toxicity probability parameter and has a probability that the efficacy rate that is greater than a final success efficacy target is greater than a final success efficacy probability parameter. In some embodiments, the criteria for stopping a study for early success are the same or different (for example, higher or more stringent) than the criteria for determining if a completed trial was a success.

In some embodiments, the final success for the trial within a cohort is defined as a success if a regimen has a greater than 50%, 55%, 60%, 65%, 70%, 75%, 80%, 85%, 90%, 95%, or greater than a 99% probability of having a low toxicity, e.g., a DLT rate of less than 40%, 355, 34%, 33%, 30%, 25%, 20%, 15%, 10%, 5%, or 1%, and a greater than 60%, 65%, 70%, 75%, 80%, 85%, 90%, 95%, or greater than 99% chance of having an efficacy rate, e.g., a CR rate, of greater than 20%, 25%, 30%, 31%, 32%, 33%, 34%, 35%, 40%, 45%, or 50%. In certain embodiments, the clinical trial is a success if a regimen has a greater than 50% probability of having a low toxicity, e.g., a DLT rate of less than 33%, and a greater than a 80% chance of having a efficacy rate, e.g., a CR rate, of greater than 25%.

L. Computer Implemented Steps

In certain embodiments, methods provided herein are computer implemented methods and/or are performed with the aid of a computer. In some embodiments, provided herein are methods for allocating subjects to treatment regimens, for example in a clinical trial, by computer implemented methods and/or by methods which include steps that are computer implemented steps. In some embodiments, calculating an overall utility score for a treatment regimen is implemented by a computer. In particular embodiments, allocating a subject to a treatment group based on the overall utility score is implemented on a computer. In certain embodiments, calculating an overall utility score and/or assigning a subject to a regimen are implemented by computer. In particular embodiments, a computer system comprising a processor and memory is provided, wherein the memory contains instructions operable to cause the processor to carry out any one or more of steps of the methods provided herein.

In certain embodiments, methods provided herein may be practiced, at least in part, with computer system configurations, including single-processor or multi-processor computer systems, minicomputers, mainframe computers, as well as personal computers, hand-held computing devices, microprocessor-based and/or programmable consumer electronics and the like, each of which may operatively communicate with one or more associated devices. In particular embodiments, the methods provided herein may be practiced, at least in part, in distributed computing environments such that certain tasks are performed by remote processing devices that are linked through a communications network. In a distributed computing environment, program modules may be located in local and/or remote memory storage devices. In particular embodiments, some or all steps of the methods provided herein may be practiced on stand-alone computers.

In particular embodiments, some or all of the steps of the methods provided herein can operate in the general context of computer-executable instructions, such as program modules, executed by one or more components. Generally, program modules include routines, programs, objects, data structures, etc., that perform particular tasks or implement particular abstract data types. Typically, the functionality of the program modules may be combined or distributed as desired. In certain embodiments, instructions operable to cause the processor to carry out any one or more steps of the methods provided herein can be embodied on a computer-readable medium having computer-executable instructions and transmitted as signals manufactured to transmit such instructions as well as the results of performing the instructions, for instance, on a network.

### III. THERAPEUTIC OUTCOMES

In the provided methods, one or more therapeutic outcomes or events associated with toxicity (toxic outcome) and one or more therapeutic outcomes or events associated with efficacy (response outcome) of the therapeutic agent is assessed and dosing decisions are made in accord with the provided methods. In some embodiments, the provided methods determine dosing options for a therapeutic agent in which the therapy is known to work fast and/or in which the response outcome occurs rapidly, e.g. a therapeutic agent that is fast acting. In some cases, such therapeutic agents include agents that are specific or substantially specific for a particular disease or condition. In some embodiments, such therapeutic agents include, for example, small molecule drugs, gene therapies, molecularly targeted agents, immunotherapies and/or cell-based therapies. Thus, unlike other therapeutic agents that may be non-specific, such as cytotoxic or chemotherapeutic agents, the ability to achieve a response outcome faster or more rapidly means that the response outcome can be determined in a time frame that is the same or that is similar to the time frame at which a toxic outcome may occur. In some embodiments, the information about toxic outcome and response outcome can be jointly assessed in a subject, such as assessed in parallel or at around the same time or substantially the same time, and used to inform the dosing decisions or adaptive treatments of subjects.

In some embodiments, the toxic outcome and response outcome are monitored at a time at which a toxicity outcome and a response outcome are present. The particular time at which such outcome may be present will depend on the particular therapeutic agent and is known to a skilled artisan, such as a physician or clinician, or is within the level of such a skilled artisan to determine. In some embodiments, the time at which a toxic outcome or response outcome is assessed is within or within about a period of time in which a symptom of toxicity or efficacy is detectable in a subject or at such time in which an adverse outcome associated with non-response or toxicity is not detectable in the subject. In some embodiments, the time period is near or substantially near to when the toxic outcome and/or response outcome has peaked in the subject.

In some embodiments, the toxic outcome or response outcome can be assessed in the subject at a time that is within or about within 120 days after initiation of the first dose of the therapeutic agent to the subject, within or within about 90 days after initiation of the first dose, within or within about 60 days after initiation of the first dose of the therapeutic agent or within or within about 30 days after initiation of the first dose to a subject. In some embodiments, the toxic outcome or response can be assessed in the subject within or within about 6 days, 12 days, 16 days, 20 days, 24 days, 28 days, 32 days, 36 days, 40 days, 44 days, 48 days, 52 days, 56 days, 60 days, 64 days, 68 days, 72 days, 76 days, 80 days, 84 days, 88 days, 92 days, 96 days or 100 days after initiation of the first dose to a subject.

In some embodiments, the toxic outcome or response outcome is present or can be assessed or monitored at such time period where only a single dose of the therapeutic agent is administered. In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the first dose is a single or continuous administration of the specified number of cells, given or initiated at a single point in time. In some contexts, however, the first dose is administered in multiple injections or infusions over a period of no more than three days, such as once a day for three days or for two days or by multiple infusions over a single day period.

The term “split dose” refers to a dose that is split so that it is administered over more than one day. This type of dosing is encompassed by the present methods and is considered to be a single dose.

As used herein, “first dose” is used to describe the timing of a given dose, which, in some cases can be the only dose or can be followed by one or more repeat or additional doses. The term does not necessarily imply that the subject has never before received a dose of a therapeutic agent even that the subject has not before received a dose of the same or substantially the same therapeutic agent.

In particular embodiments, subjects that are assigned to a regimen are monitored and/or assessed to determine if the subject will experience a toxic outcome during or after treatment with the therapeutic agent. In some embodiments, the toxic outcome is a dose limiting toxicity. In some embodiments, the toxic outcome or response outcome is present and/or can be assessed or monitored at such time period that is after a first cycle of administration of the therapeutic agent, after a second cycle of administration of the therapeutic agent, after a third cycle of administration of the therapeutic agent, or after a fourth cycle of administration of the therapeutic agent. In some embodiments, a cycle of administration can be a repeated schedule of a dosing regimen that is repeated over successive administrations. In some embodiments, a schedule of administration can be daily, every other days, or once a week for one week, two weeks, three weeks or four weeks (e.g. 28 days). In some embodiments, a cycle of administration can be tailored to add periods of discontinued treatment in order to provide a rest period from exposure to the agent. In some embodiments, the length of time for the discontinuation of treatment can be for a predetermined time or can be empirically determined depending on how the patient is responding or depending on observed side effects. For example, the treatment can be discontinued for one week, two weeks, one month or several months.

In some embodiments, the toxic outcome and response outcome can be assessed by monitoring one or more symptoms or events associated with a toxic outcome and one or more symptoms or events associated with a response outcome. In some embodiments, the disease or condition is a tumor or cancer.

A. Toxic Outcome

In some embodiments, a toxic outcome in a subject to administration of a therapeutic agent (e.g. CAR T-cells) can be assessed or monitored. In particular embodiments, subjects assigned to a treatment regimen, for example in a clinical trial, are assessed and/or monitored for a period of time to determine if the subjects experience a toxic outcome. In particular embodiments, the subjects that are assigned to a treatment regimen are assessed and/or monitored to determine the toxicity rate of the treatment regimen. In certain embodiments, the toxic outcome is a dose limiting toxicity (DLT). In particular embodiments, the toxicity rate is the DLT rate. In some embodiments, the toxic outcome is or is associated with the presence of a toxic event, such as cytokine release syndrome (CRS), severe CRS (sCRS), macrophage activation syndrome, tumor lysis syndrome, fever of at least at or about 38 degrees Celsius for three or more days and a plasma level of CRP of at least at or about 20 mg/dL, neurotoxicity and/or neurotoxicity. In some embodiments, the toxic outcome is a sign, or symptom, particular signs, and symptoms and/or quantities or degrees thereof which presence or absence may specify a particular extent, severity or level of toxicity in a subject. It is within the level of a skilled artisan to specify or determine a particular sign, symptom and/or quantities or degrees thereof that are related to an undesired toxic outcome of a therapeutic agent (e.g. CAR-T cells).

In some embodiments, the toxic outcome is an indicator associated with the toxic event. In certain embodiments, the toxic outcome is a DLT and is an indicator associated with the toxic event. In some embodiments, the toxic outcome is the presence or absence of one or more biomarkers or the presence of absence of a level of one or more biomarkers. In some embodiments, the biomarker is molecule present in the serum or other bodily fluid or tissue indicative of cytokine-release syndrome (CRS), severe CRS or CRS-related outcomes. In some embodiments, the biomarker is a molecule present in the serum or other bodily fluid or tissue indicative of neurotoxicity or severe neurotoxicity.

In some embodiments, the subject exhibits toxicity or a toxic outcome, e.g., a DLT, if a toxic event, such as CRS-related outcomes, e.g. if a serum level of an indicator of CRS or other biochemical indicator of the toxicity is more than at or about 10 times, more than at or about 15 times, more than at or about 20 times, more than at or about 25 times, more than at or about 50 times, more than at or about 75 times, more than at or about 100 times, more than at or about 125 times, more than at or about 150 times, more than at or about 200 times, or more than at or about 250 times the baseline or pre-treatment level, such as the serum level of the indicator immediately prior to administration of the first dose of the therapeutic agent. In some embodiments, a DLT is a toxic event that is a CRS-related outcome.

Exemplary signs or symptoms associated with CRS include fever, rigors, chills, hypotension, dyspnea, acute respiratory distress syndrome (ARDS), encephalopathy, ALT/AST elevation, renal failure, cardiac disorders, hypoxia, neurologic disturbances, and death. Neurological complications include delirium, seizure-like activity, confusion, word-finding difficulty, aphasia, and/or becoming obtunded. Other CRS-related signs or outcomes include fatigue, nausea, headache, seizure, tachycardia, myalgias, rash, acute vascular leak syndrome, liver function impairment, and renal failure. In some aspects, CRS is associated with an increase in one or more factors such as serum-ferritin, d-dimer, aminotransferases, lactate dehydrogenase and triglycerides, or with hypofibrinogenemia or hepatosplenomegaly.

In some embodiments, signs or symptoms associated with CRS include one or more of: persistent fever, e.g., fever of a specified temperature, e.g., greater than at or about 38 degrees Celsius, for two or more, e.g., three or more, e.g., four or more days or for at least three consecutive days; fever greater than at or about 38 degrees Celsius; elevation of cytokines (e.g. IFNγ or IL-6); and/or at least one clinical sign of toxicity, such as hypotension (e.g., as measured by at least one intravenous vasoactive pressor); hypoxia (e.g., plasma oxygen (PO₂) levels of less than at or about 90%); and/or one or more neurologic disorders (including mental status changes, obtundation, and seizures). In certain embodiments, a DLT is a sign or symptom associated with CRS.

In some embodiments, the presence of one or more biomarkers is indicative of the grade of, severity or extent of a toxic event, e.g., a DLT, such as CRS or neurotoxicity. In some embodiments, the toxic outcome is a particular grade, severity or extent of a toxic event, such as a particular grade, severity or extent of CRS or neurotoxicity. In some embodiments, the presence of a toxic event about a certain grade, severity or extent can be a dose-limiting toxicity. In some embodiments, the absence of a toxic event or the presence of a toxic event below a certain grade, severity or extent can indicate the absence of a dose-limiting toxicity. In particular embodiments, the presence of one or more biomarkers is indicative of a DLT.

CRS criteria that appear to correlate with the onset of CRS to predict which patients are more likely to be at risk for developing sCRS have been developed (see Davilla et al. Science translational medicine. 2014; 6(224):224ra25). Factors include fevers, hypoxia, hypotension, neurologic changes, elevated serum levels of inflammatory cytokines whose treatment-induced elevation can correlate well with both pretreatment tumor burden and sCRS symptoms. Other guidelines on the diagnosis and management of CRS are known (see e.g., Lee et al, Blood. 2014; 124(2):188-95). In some embodiments, the criteria reflective of CRS grade are those detailed in Table 1 below.

TABLE 1
Exemplary Grading Criteria for CRS

| Grade | Description of Symptoms |
|---|---|
| 1 Mild | Not life-threatening, require only symptomatic treatment |
| | such as antipyretics and anti-emetics (e.g., fever, nausea, |
| | fatigue, headache, myalgias, malaise) |
| 2 Moderate | Require and respond to moderate intervention: |
| | Oxygen requirement <40%, or |
| | Hypotension responsive to fluids or low dose of a single |
| | vasopressor, or |
| | Grade 2 organ toxicity (by CTCAE v4.0) |
| 3 Severe | Require and respond to aggressive intervention: |
| | Oxygen requirement ≥40%, or |
| | Hypotension requiring high dose of a single vasopressor |
| | (e.g., norepinephrine ≥20 μg/kg/min, dopamine ≥10 |
| | μg/kg/min, phenylephrine ≥200 μg/kg/min, or |
| | epinephrine ≥10 μg/kg/min), or |
| | Hypotension requiring multiple vasopressors (e.g., |
| | vasopressin + one of the above agents, or combination |
| | vasopressors equivalent to ≥20 μg/kg/min norepinephrine), |
| | or |
| | Grade 3 organ toxicity or Grade 4 transaminitis (by CTCAE |
| | v4.0) |
| 4 Life- | Life-threatening: |
| threatening | Requirement for ventilator support, or |
| | Grade 4 organ toxicity (excluding transaminitis) |
| 5 Fatal | Death |

In some embodiments, the toxic outcome is severe CRS. In some embodiments, the toxic outcome is the absence of severe CRS (e.g. moderate or mild CRS). In some embodiments, severe CRS includes CRS with a grade of 3 or greater, such as set forth in Table 1. In some embodiments, the level of the toxic outcome, e.g. the CRS-related outcome, e.g. the serum level of an indicator of CRS, is measured by ELISA. In some embodiments, fever and/or levels of CRP can be measured. In some embodiments, subjects with a fever and a CRP≥15 mg/dL may be considered high-risk for developing severe CRS.

In some embodiments, a DLT is severe CRS. In some embodiments, the DLT is severe CRS that includes CRS with a grade of 3 or greater, such as set forth in Table 1. In particular embodiments, a DLT is a CRS that is grade 2 or greater, grade 3 or greater, grade 4 or greater, or grade 5.

In some aspects, the toxic outcome is or is associated with neurotoxicity. In certain embodiments, a DLT is or is associated with neurotoxicity. In some embodiments, signs or symptoms associated with a clinical risk of neurotoxicity include confusion, delirium, expressive aphasia, obtundation, myoclonus, lethargy, altered mental status, convulsions, seizure-like activity, seizures (optionally as confirmed by electroencephalogram [EEG]), elevated levels of beta amyloid (Aβ), elevated levels of glutamate, and elevated levels of oxygen radicals. In some embodiments, neurotoxicity is graded based on severity (e.g., using a Grade 1-5 scale (see, e.g., Guido Cavaletti & Paola Marmiroli Nature Reviews Neurology 6, 657-666 (December 2010); National Cancer Institute—Common Toxicity Criteria version 4.03 (NCI-CTCAE v4.03). In some embodiments, severe neurotoxicity includes neurotoxicity with a grade of 3 or greater, such as set forth in Table 2.

TABLE 2
Exemplary Grading Criteria for neurotoxicity

| Grade | Description of Symptoms |
|---|---|
| 1 Asymptomatic | Mild or asymptomatic symptoms |
| or Mild | |
| 2 Moderate | Presence of symptoms that limit instrumental activities |
| | of daily living (ADL), such as preparing meals, |
| | shopping for groceries or clothes, using the telephone, |
| | managing money |
| 3 Severe | Presence of symptoms that limit self-care ADL, such |
| | as bathing, dressing and undressing, feeding self, using |
| | the toilet, taking medications |
| 4 Life- | Symptoms that are life-threatening, requiring urgent |
| threatening | intervention |
| 5 Fatal | Death |

In certain embodiments, the DLT is or is associated with neurotoxicity. In some embodiments, the DLT is neurotoxicity of grade 2 or greater, grade 3 or greater, grade 4 or greater, or grade 5. In some embodiments, the DLT is or is associated with neurotoxicity of grade 3 or greater.

In some embodiments, the toxic outcome is a dose-limiting toxicity. In some embodiments, the toxic outcome is the absence of a dose-limiting toxicity. In some embodiments, a dose-limiting toxicity (DLT) is defined as any grade 3 or higher toxicity as assessed by any known or published guidelines for assessing the particular toxicity, such as any described above and including the National Cancer Institute (NCI) Common Terminology Criteria for Adverse Events (CTCAE) version 4.0.

B. Response Outcome

In some embodiments, a response outcome in a subject to administration of a therapeutic agent can be monitored and/or assessed. In particular embodiments, subjects assigned to a treatment regimen, for example a treatment regimen in a clinical trial, are assessed and/or monitored for a period of time to determine if the subjects experience a response. In particular embodiments, the subjects that are assigned to a treatment regimen are assessed and/or monitored to determine the rate of a response, e.g., an efficacy rate, of the treatment regimen. In certain embodiments, the response outcome is a complete response (CR). In particular embodiments, the efficacy rate is the CR rate. In some embodiments, the response outcome is no response. In some embodiments, the response outcome is a partial response. In some embodiments, the response outcome is a complete response (CR). In some embodiments, response outcome is assessed by monitoring the disease burden in the subject. In some embodiments, the presence of no response, a partial response or a clinical or complete response can be assessed.

In some embodiments, a partial response or complete response is one in which the therapeutic agent reduces or prevents the expansion or burden of the disease or condition in the subject. For example, where the disease or condition is a tumor, reduced disease burden exists or is present if there is a reduction in the tumor size, bulk, metastasis, percentage of blasts in the bone marrow or molecularly detectable cancer and/or an improvement prognosis or survival or other symptom associated with tumor burden compared to prior to treatment with the therapeutic agent (e.g. CAR T cells). In particular embodiments, the efficacy rate is the rate of subjects, for example in a treatment regimen, that experience a partial response or a complete response.

In some embodiments, the disease or condition is a tumor and a reduction in disease burden is a reduction in tumor size. In some embodiments, the disease burden reduction is indicated by a reduction in one or more factors, such as load or number of disease cells in the subject or fluid or organ or tissue thereof, the mass or volume of a tumor, or the degree or extent of metastases. In some embodiments, disease burden, e.g. tumor burden, can be assessed or monitored for the extent of morphological disease and/or minimal residual disease. In some embodiments, the efficacy rate is the rate of subjects, for example in a treatment regimen, that experience a reduction in disease burden.

In some embodiments, the burden of a disease or condition in the subject is detected, assessed, or measured. Disease burden may be detected in some aspects by detecting the total number of disease or disease-associated cells, e.g., tumor cells, in the subject, or in an organ, tissue, or bodily fluid of the subject, such as blood or serum. In some embodiments, disease burden, e.g. tumor burden, is assessed by measuring the mass of a solid tumor and/or the number or extent of metastases. In some aspects, survival of the subject, survival within a certain time period, extent of survival, presence or duration of event-free or symptom-free survival, or relapse-free survival, is assessed. In some embodiments, any symptom of the disease or condition is assessed. In some embodiments, the measure of disease or condition burden is specified. In certain embodiments, the burden or a disease or condition is detected, assessed, and/or measured in subjects in a treatment regimen to determine the efficacy rate of the treatment regimen.

In some embodiments, disease burden can encompass a total number of cells of the disease in the subject or in an organ, tissue, or bodily fluid of the subject, such as the organ or tissue of the tumor or another location, e.g., which would indicate metastasis. For example, tumor cells may be detected and/or quantified in the blood or bone marrow in the context of certain hematological malignancies. Disease burden can include, in some embodiments, the mass of a tumor, the number or extent of metastases and/or the percentage of blast cells present in the bone marrow.

In some embodiments, a subject has leukemia. The extent of disease burden can be determined by assessment of residual leukemia in blood or bone marrow. In particular embodiments, residual leukemia in blood or bone marrow is detected, assessed, and/or measured in subjects of a treatment regimen to determine the efficacy rate of the treatment regimen.

In some embodiments, a response outcome exists if there is a reduction in the percent of blasts in the bone marrow compared to the percent of blasts in the bone marrow prior to treatment with the therapeutic agent. In some embodiments, reduction of disease burden exists if there is a decrease or reduction of at least or at least about 20%, 30%, 40%, 50%, 60%, 70%, 80%, 90%, 95% or more in the number or percentage of blasts in the bone marrow compared to the number or percent of blasts in the bone marrow prior to treatment. In particular embodiments, the percent of blasts in the bone marrow compared to the percent of blasts in the bone marrow prior to treatment with the therapeutic agent is detected, assessed, and/or measured in subjects in a treatment regimen to determine the efficacy rate of the treatment regimen.

In some embodiments, the subject exhibits a response if the subject does not exhibit morphologic disease (non-morphological disease) or does not exhibit substantial morphologic disease. In some embodiments, a subject exhibits morphologic disease if there are greater than or equal to 5% blasts in the bone marrow, for example, as detected by light microscopy. In some embodiments, a subject exhibits complete or clinical remission if there are less than 5% blasts in the bone marrow. In particular embodiments, morphological disease is detected, assessed, and/or measured in subjects of a treatment regimen to determine the efficacy rate of the regimen.

In some embodiments, a subject exhibits reduced or decreased disease burden if they exhibited morphological disease prior to treatment and exhibit complete remission (e.g., fewer than 5% blasts in bone marrow) with or without molecular disease (e.g., minimum residual disease (MRD) that is molecularly detectable, e.g., as detected by flow cytometry or quantitative PCR) after treatment. In some embodiments, a subject exhibits reduced or decreased disease burden if they exhibited molecular disease prior to treatment and do not exhibit molecular disease after treatment. In certain embodiments, the efficacy rate of a treatment regimen is determined by quantifying the number of subjects of the treatment regimen that exhibit reduced or decreased disease burden and/or exhibit complete remission.

In some embodiments, a subject may exhibit complete remission, but a small proportion of morphologically undetectable (by light microscopy techniques) residual leukemic cells are present. A subject is said to exhibit minimum residual disease (MRD) if the subject exhibits less than 5% blasts in the bone marrow and exhibits molecularly detectable cancer. In some embodiments, molecularly detectable cancer can be assessed using any of a variety of molecular techniques that permit sensitive detection of a small number of cells. In some aspects, such techniques include PCR assays, which can determine unique Ig/T-cell receptor gene rearrangements or fusion transcripts produced by chromosome translocations. In some embodiments, flow cytometry can be used to identify cancer cell based on leukemia-specific immunophenotypes. In some embodiments, molecular detection of cancer can detect as few as 1 leukemia or blast cell in 100,000 normal cells or 1 leukemia or blast cell in 10,000 normal cells. In some embodiments, a subject exhibits MRD that is molecularly detectable if at least or greater than 1 leukemia cell in 100,000 cells is detected, such as by PCR or flow cytometry.

In some embodiments, the disease burden of a subject is molecularly undetectable or MRD⁻, such that, in some cases, no leukemia cells are able to be detected in the subject using PCR or flow cytometry techniques.

In some embodiments the response outcome is the absence of a CR or the presence of a complete response in which the subject achieves or exhibits minimal residual disease or molecular detectable disease status. In some embodiments, the response outcome is the presence of a CR with molecularly detectable disease or the presence of a CR without molecularly detectable disease. In some embodiments, subjects are assessed for disease burden using methods as described herein, such as methods that assess blasts in bone marrow or molecular disease by flow cytometry or qPCR methods.

### IV. ADMINISTERING A THERAPEUTIC AGENT

In some embodiments, the provided methods can be used for determining dosing actions or adapting dosing regimens for administering a therapeutic agent. In some embodiments, the therapeutic agent is specific or substantially specific for or acts preferentially on or is targeted against a disease or condition. In some embodiments, the therapeutic agent is one that, compared to conventional chemotherapeutic or cytotoxic agents, exhibits a fast or rapid response. In some embodiments, the therapeutic agent is a molecularly targeted agent, an immunotherapy and/or a cell therapy. In some embodiments, the therapeutic agent is a small molecule, a nucleic acid, a peptide or is a polypeptide or protein. In certain embodiments, the agent is not a chemotherapeutic agent. In particular embodiments, the agent is not a cytotoxic agent. In some embodiments, the therapeutic agent is a targeted antibody therapy, such as a bispecific antibody therapy, including an anti-CD3 bispecific antibody therapy (e.g. Blinatumomab). In some embodiments, the therapeutic agent is a checkpoint inhibitor, such as an anti-checkpoint antibody, including an anti-PD-L1 antibody, anti-PD-1 antibody, or anti-CTLA-4 antibody. In some embodiments, the therapeutic agent is an adoptive cell therapy, such as any T cell therapy, for example, a tumor infiltrating lymphocytic (TIL) therapy, a transgenic TCR therapy or a chimeric antigen receptor (CAR)-expressing T cell therapy.

In some embodiments, the methods comprise designating two or more possible unique treatment regimens for administering a therapeutic agent to a population of subjects enrolled in a clinical trial.

A. Adoptive Cell Therapy

1. Cells

In certain embodiments, the therapeutic agent is a cell therapy. The cells generally are eukaryotic cells, such as mammalian cells, and typically are human cells, e.g., those derived from human subjects and engineered, for example, to express the recombinant receptors. In some embodiments, the cells are derived from the blood, bone marrow, lymph, or lymphoid organs, are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods include off-the-shelf methods. In some aspects, such as for off-the-shelf technologies, the cells are pluripotent and/or multipotent, such as stem cells, such as induced pluripotent stem cells (iPSCs). In some embodiments, the methods include isolating cells from the subject, preparing, processing, culturing, and/or engineering them, and re-introducing them into the same subject, before or after cryopreservation.

Among the sub-types and subpopulations of T cells and/or of CD4+ and/or of CD8+ T cells are naïve T (T_{N}) cells, effector T cells (T_{EFF}), memory T cells and sub-types thereof, such as stem cell memory T (T_{SCM}), central memory T (T_{CM}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are natural killer (NK) cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

a. Vectors and Methods for Genetic Engineering

In some embodiments, the cells expressing a recombinant receptor are produced by the genetically engineered cells expressing recombinant receptors. The genetic engineering generally involves introduction of a nucleic acid encoding the recombinant or engineered component into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some contexts, overexpression of a stimulatory factor (for example, a lymphokine or a cytokine) may be toxic to a subject. Thus, in some contexts, the engineered cells include gene segments that cause the cells to be susceptible to negative selection in vivo, such as upon administration in adoptive immunotherapy. For example in some aspects, the cells are engineered so that they can be eliminated as a result of a change in the in vivo condition of the subject to which they are administered. The negative selectable phenotype may result from the insertion of a gene that confers sensitivity to an administered agent, for example, a compound. Negative selectable genes include the Herpes simplex virus type I thymidine kinase (HSV-I TK) gene (Wigler et al., Cell II: 223, 1977) which confers ganciclovir sensitivity; the cellular hypoxanthine phosphribosyltransferase (HPRT) gene, the cellular adenine phosphoribosyltransferase (APRT) gene, bacterial cytosine deaminase, (Mullen et al., Proc. Natl. Acad. Sci. USA. 89:33 (1992)).

In some aspects, the cells further are engineered to promote expression of cytokines or other factors. Various methods for the introduction of genetically engineered components, e.g., antigen receptors, e.g., CARs, are well known and may be used with the provided methods and compositions. Exemplary methods include those for transfer of nucleic acids encoding the receptors, including via viral, e.g., retroviral or lentiviral, transduction, transposons, and electroporation.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

b. Preparation of Cells for Engineering

In some embodiments, preparation of the engineered cells includes one or more culture and/or preparation steps. The cells for introduction of the nucleic acid encoding the transgenic receptor such as the CAR, may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some aspects, the cells of the second dose are derived from the same apheresis product as the cells of the first dose. In some embodiments, the cells of multiple doses, e.g., first, second, third, and so forth, are derived from the same apheresis product.

In other embodiments, the cells of the second (or other subsequent) dose are derived from an apheresis product that is distinct from that from which the cells of the first (or other prior) dose are derived.

In some embodiments, the cells are derived from cell lines, e.g., T cell lines. The cells in some embodiments are obtained from a xenogeneic source, for example, from mouse, rat, non-human primate, and pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28⁺, CD62L⁺, CCR7⁺, CD27⁺, CD127⁺, CD4⁺, CD8⁺, CD45RA⁺, and/or CD45RO⁺ T cells, are isolated by positive or negative selection techniques.

For example, CD3⁺, CD28⁺ T cells can be positively selected using CD3/CD28 conjugated magnetic beads (e.g., DYNABEADS® M-450 CD3/CD28 T Cell Expander).

In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker⁺) at a relatively higher level (marker^{high}) on the positively or negatively selected cells, respectively.

In some embodiments, T cells are separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

In some embodiments, CD8⁺ cells are further enriched for or depleted of naive, central memory, effector memory, and/or central memory stem cells, such as by positive or negative selection based on surface antigens associated with the respective subpopulation. In some embodiments, enrichment for central memory T (T_{cM}) cells is carried out to increase efficacy, such as to improve long-term survival, expansion, and/or engraftment following administration, which in some aspects is particularly robust in such sub-populations. See Terakura et al. (2012) Blood. 1:72-82; Wang et al. (2012) J Immunother. 35(9):689-701. In some embodiments, combining T_{cM}-enriched CD8⁺ T cells and CD4⁺ T cells further enhances efficacy.

In embodiments, memory T cells are present in both CD62L⁺ and CD62L⁻ subsets of CD8⁺ peripheral blood lymphocytes. PBMC can be enriched for or depleted of CD62L⁻CD8⁺ and/or CD62L⁺CD8⁺ fractions, such as using anti-CD8 and anti-CD62L antibodies.

In some embodiments, the enrichment for central memory T (T_{CM}) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8⁺ population enriched for T_{cM} cells is carried out by depletion of cells expressing CD4, CD14, CD45RA, and positive selection or enrichment for cells expressing CD62L. In one aspect, enrichment for central memory T (T_{CM}) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8⁺ cell population or subpopulation, also is used to generate the CD4⁺ cell population or subpopulation, such that both the positive and negative fractions from the CD4-based separation are retained and used in subsequent steps of the methods, optionally following one or more further positive or negative selection steps.

In a particular example, a sample of PBMCs or other white blood cell sample is subjected to selection of CD4⁺ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4⁺ T helper cells are sorted into naïve, central memory, and effector cells by identifying cell populations that have cell surface antigens. CD4⁺ lymphocytes can be obtained by standard methods. In some embodiments, naive CD4⁺ T lymphocytes are CD45RO⁻, CD45RA⁺, CD62L⁺, CD4⁺ T cells. In some embodiments, central memory CD4⁺ cells are CD62L⁺ and CD45RO⁺. In some embodiments, effector CD4⁺ cells are CD62L⁻ and CD45RO⁻.

In one example, to enrich for CD4⁺ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher© Humana Press Inc., Totowa, N.J.).

In some aspects, the sample or composition of cells to be separated is incubated with small, magnetizable or magnetically responsive material, such as magnetically responsive particles or microparticles, such as paramagnetic beads (e.g., such as Dynalbeads or MACS beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select. Exemplary methods for cell selection are described in International Patent Application Publication Numbers WO2015157384 and/or WO 2015/164675, which are incorporated by reference in their entirety, all or a portion of which could be used in connection with the methods described herein.

In some embodiments, the magnetic particle or bead comprises a magnetically responsive material bound to a specific binding member, such as an antibody or other binding partner. There are many well-known magnetically responsive materials used in magnetic separation methods. Suitable magnetic particles include those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 are other examples.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, and magnetizable particles or antibodies conjugated to cleavable linkers. In some embodiments, the magnetizable particles are biodegradable.

In some embodiments, the affinity-based selection is via magnetic-activated cell sorting (MACS) (Miltenyi Biotech, Auburn, Calif.). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In certain embodiments, the isolation or separation is carried out using a system, device, or apparatus that carries out one or more of the isolation, cell preparation, separation, processing, incubation, culture, and/or formulation steps of the methods. In some aspects, the system is used to carry out each of these steps in a closed or sterile environment, for example, to minimize error, user handling and/or contamination. In one example, the system is a system as described in International Patent Application, Publication Number WO2009/072003, or US 20110003380 A1. In some embodiments, the isolation or separation is carried out according to methods described in International Patent Application Publication Number WO 2015/164675, the contents of which are incorporated by reference in their entirety.

In some embodiments, the system or apparatus carries out one or more, e.g., all, of the isolation, processing, engineering, and formulation steps in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some aspects, the separation and/or other steps is carried out using CliniMACS system (Miltenyi Biotic), for example, for automated separation of cells on a clinical-scale level in a closed and sterile system. Components can include an integrated microcomputer, magnetic separation unit, peristaltic pump, and various pinch valves. The integrated computer in some aspects controls all components of the instrument and directs the system to perform repeated procedures in a standardized sequence. The magnetic separation unit in some aspects includes a movable permanent magnet and a holder for the selection column. The peristaltic pump controls the flow rate throughout the tubing set and, together with the pinch valves, ensures the controlled flow of buffer through the system and continual suspension of cells.

The CliniMACS system in some aspects uses antibody-coupled magnetizable particles that are supplied in a sterile, non-pyrogenic solution. In some embodiments, after labelling of cells with magnetic particles the cells are washed to remove excess particles. A cell preparation bag is then connected to the tubing set, which in turn is connected to a bag containing buffer and a cell collection bag. The tubing set consists of pre-assembled sterile tubing, including a pre-column and a separation column, and are for single use only. After initiation of the separation program, the system automatically applies the cell sample onto the separation column. Labelled cells are retained within the column, while unlabeled cells are removed by a series of washing steps. In some embodiments, the cell populations for use with the methods described herein are unlabeled and are not retained in the column. In some embodiments, the cell populations for use with the methods described herein are labeled and are retained in the column. In some embodiments, the cell populations for use with the methods described herein are eluted from the column after removal of the magnetic field, and are collected within the cell collection bag.

In certain embodiments, separation and/or other steps are carried out using the CliniMACS Prodigy system (Miltenyi Biotec). The CliniMACS Prodigy system in some aspects is equipped with a cell processing unity that permits automated washing and fractionation of cells by centrifugation. The CliniMACS Prodigy system can also include an onboard camera and image recognition software that determines the optimal cell fractionation endpoint by discerning the macroscopic layers of the source cell product. For example, peripheral blood is automatically separated into erythrocytes, white blood cells and plasma layers. The CliniMACS Prodigy system can also include an integrated cell cultivation chamber which accomplishes cell culture protocols such as, e.g., cell differentiation and expansion, antigen loading, and long-term cell culture. Input ports can allow for the sterile removal and replenishment of media and cells can be monitored using an integrated microscope. See, e.g., Klebanoff et al. (2012) J Immunother. 35(9): 651-660, Terakura et al. (2012) Blood. 1:72-82, and Wang et al. (2012) J Immunother 35(9):689-701.

In some embodiments, a cell population described herein is collected and enriched (or depleted) via flow cytometry, in which cells stained for multiple cell surface markers are carried in a fluidic stream. In some embodiments, a cell population described herein is collected and enriched (or depleted) via preparative scale (FACS)-sorting. In certain embodiments, a cell population described herein is collected and enriched (or depleted) by use of microelectromechanical systems (MEMS) chips in combination with a FACS-based detection system (see, e.g., WO 2010/033140, Cho et al. (2010) Lab Chip 10, 1567-1573; and Godin et al. (2008) J Biophoton. 1(5):355-376. In both cases, cells can be labeled with multiple markers, allowing for the isolation of well-defined T cell subsets at high purity.

In some embodiments, the antibodies or binding partners are labeled with one or more detectable marker, to facilitate separation for positive and/or negative selection. For example, separation may be based on binding to fluorescently labeled antibodies. In some examples, separation of cells based on binding of antibodies or other binding partners specific for one or more cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously.

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701. In some aspects, incubation is carried out using a system, device, apparatus, and/or method as described in International Patent Application Publication Number WO2016/073602 or US 2016/0122782 the contents of which are incorporated by reference in their entirety. In some embodiments, the incubation and/or culturing is carried out according to methods described in International Patent Application Publication Number WO 2015/164675, the contents of which are incorporated by reference in their entirety.

In some embodiments, the T cells are expanded by adding to the culture-initiating composition feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). In some aspects, the non-dividing feeder cells can comprise gamma-irradiated PBMC feeder cells. In some embodiments, the PBMC are irradiated with gamma rays in the range of about 3000 to 3600 rads to prevent cell division. In some aspects, the feeder cells are added to culture medium prior to the addition of the populations of T cells.

In some embodiments, the stimulating conditions include temperature suitable for the growth of human T lymphocytes, for example, at least about 25 degrees Celsius, generally at least about 30 degrees, and generally at or about 37 degrees Celsius. Optionally, the incubation may further comprise adding non-dividing EBV-transformed lymphoblastoid cells (LCL) as feeder cells. LCL can be irradiated with gamma rays in the range of about 6000 to 10,000 rads. The LCL feeder cells in some aspects is provided in any suitable amount, such as a ratio of LCL feeder cells to initial T lymphocytes of at least about 10:1.

In embodiments, antigen-specific T cells, such as antigen-specific CD4+ and/or CD8+ T cells, are obtained by stimulating naive or antigen specific T lymphocytes with antigen. For example, antigen-specific T cell lines or clones can be generated to cytomegalovirus antigens by isolating T cells from infected subjects and stimulating the cells in vitro with the same antigen.

2. Recombinant Receptors Expressed by the Cells

The cells generally express recombinant receptors. The receptors may include antigen receptors, such as functional non-TCR antigen receptors, including chimeric antigen receptors (CARs), and other antigen-binding receptors such as transgenic T cell receptors (TCRs). The receptors may also include other chimeric receptors, such as receptors binding to particular ligands and having transmembrane and/or intracellular signaling domains similar to those present in a CAR.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also International Patent Publication No.: WO2014031687, U.S. Pat. Nos. 8,339,645, 7,446,179, 7,446,190, and 8,389,282, and U.S. patent application Publication No. US 2013/0149337. Among the chimeric receptors are chimeric antigen receptors (CARs). The chimeric receptors, such as CARs, generally include an extracellular antigen binding domain, such as a portion of an antibody molecule, generally a variable heavy (VH) chain region and/or variable light (VL) chain region of the antibody, e.g., an scFv antibody fragment.

In some embodiments, the binding domain(s), e.g., the antibody, e.g., antibody fragment, portion of the recombinant receptor further includes at least a portion of an immunoglobulin constant region, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers, e.g., hinge regions, include those described in international patent application publication number WO2014031687. In some examples, the spacer is or is about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153, international patent application publication number WO2014031687, U.S. Pat. No. 8,822,647 or published app. No. US2014/0271635. In some embodiments, the constant region or portion is of a human IgG, such as IgG4 or IgG1.

This antigen recognition domain generally is linked to one or more intracellular signaling components, such as signaling components that mimic activation through an antigen receptor complex, such as a TCR complex, and/or signal via another cell surface receptor. The signal may be immunostimulatory and/or costimulatory in some embodiments. In some embodiments, it may be suppressive, e.g., immunosuppressive. Thus, in some embodiments, the antigen-binding component (e.g., antibody) is linked to one or more transmembrane and intracellular signaling domains. In some embodiments, the transmembrane domain is fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 epsilon, CD45, CD4, CD5, CD8, CD9, CD 16, CD22, CD33, CD37, CD64, CD80, CD86, CD 134, CD137, CD 154 and/or transmembrane regions containing functional variants thereof such as those retaining a substantial portion of the structural, e.g., transmembrane, properties thereof. In some embodiments, the transmembrane domain is a transmembrane domain derived from CD4, CD28, or CD8, e.g., CD8alpha, or functional variant thereof. The transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. For example, in some aspects, the CAR or other chimeric receptor includes a chimeric molecule between CD3-zeta (CD3-ζ) or Fc receptor γ and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence derived from a signaling molecule or domain that promotes primary activation of a TCR complex in a natural setting. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from the CD3 zeta chain, FcR gamma, CD3 gamma, CD3 delta and CD3 epsilon. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, DAP10, and ICOS. In some aspects, the same CAR includes both the activating and costimulatory components.

In some embodiments, the activating domain is included within one CAR, whereas the costimulatory component is provided by another CAR recognizing another antigen, present on the same cell. In some embodiments, the CARs include activating or stimulatory CARs, costimulatory CARs, both expressed on the same cell (see WO2014/055668). In some aspects, the cells include one or more stimulatory or activating CAR and/or a costimulatory CAR. In some embodiments, the cells further include inhibitory CARs (iCARs, see Fedorov et al., Sci. Transl. Medicine, 5(215) (December, 2013), such as a CAR recognizing an antigen other than the one associated with and/or specific for the disease or condition whereby an activating signal delivered through the disease-targeting CAR is diminished or inhibited by binding of the inhibitory CAR to its ligand, e.g., to reduce off-target effects.

In some embodiments, the intracellular signaling component of the recombinant receptor, such as CAR, comprises a CD3 zeta intracellular domain and a costimulatory signaling region. In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments, the CAR or other antigen receptor further includes a marker, such as a cell surface marker, which may be used to confirm transduction or engineering of the cell to express the receptor, such as a truncated version of a cell surface receptor, such as truncated EGFR (tEGFR). In some aspects, the marker includes all or part (e.g., truncated form) of CD34, a NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A. For example, a marker, and optionally a linker sequence, can be any as disclosed in published patent application No. WO2014031687. For example, the marker can be a truncated EGFR (tEGFR) that is, optionally, linked to a linker sequence, such as a T2A cleavable linker sequence.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR is one that includes multiple costimulatory domains of different costimulatory receptors.

In some embodiments, the chimeric antigen receptor includes an extracellular portion containing an antigen-binding domain, such as an antibody or antigen-binding antibody fragment, such as an scFv or Fv. In some aspects, the chimeric antigen receptor includes an extracellular portion containing the antibody or fragment and an intracellular signaling domain. In some embodiments, the antibody or fragment includes an scFv and the intracellular domain contains an ITAM. In some aspects, the intracellular signaling domain includes a signaling domain of a zeta chain of a CD3-zeta (CD3) chain. In some embodiments, the chimeric antigen receptor includes a transmembrane domain linking the extracellular domain and the intracellular signaling domain. In some aspects, the transmembrane domain contains a transmembrane portion of CD28. In some embodiments, the chimeric antigen receptor contains an intracellular domain of a T cell costimulatory molecule. The extracellular domain and transmembrane domain can be linked directly or indirectly. In some embodiments, the extracellular domain and transmembrane are linked by a spacer, such as any described herein. In some embodiments, the receptor contains extracellular portion of the molecule from which the transmembrane domain is derived, such as a CD28 extracellular portion. In some embodiments, the chimeric antigen receptor contains an intracellular domain derived from a T cell costimulatory molecule or a functional variant thereof, such as between the transmembrane domain and intracellular signaling domain. In some aspects, the T cell costimulatory molecule is CD28 or 41BB.

For example, in some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the chimeric antigen receptor contains an intracellular domain of a T cell costimulatory molecule. In some aspects, the T cell costimulatory molecule is CD28 or 41BB.

For example, in some embodiments, the CAR includes an antibody such as an antibody fragment, including scFvs, a spacer, such as a spacer containing a portion of an immunoglobulin molecule, such as a hinge region and/or one or more constant regions of a heavy chain molecule, such as an Ig-hinge containing spacer, a transmembrane domain containing all or a portion of a CD28-derived transmembrane domain, a CD28-derived intracellular signaling domain, and a CD3 zeta signaling domain. In some embodiments, the CAR includes an antibody or fragment, such as scFv, a spacer such as any of the Ig-hinge containing spacers, a CD28-derived transmembrane domain, a 4-1BB-derived intracellular signaling domain, and a CD3 zeta-derived signaling domain.

In some embodiments, nucleic acid molecules encoding such CAR constructs further includes a sequence encoding a T2A ribosomal skip element and/or a tEGFR sequence, e.g., downstream of the sequence encoding the CAR. In some embodiments, T cells expressing an antigen receptor (e.g. CAR) can also be generated to express a truncated EGFR (EGFRt) as a non-immunogenic selection epitope (e.g. by introduction of a construct encoding the CAR and EGFRt separated by a T2A ribosome switch to express two proteins from the same construct), which then can be used as a marker to detect such cells (see e.g. U.S. Pat. No. 8,802,374).

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

3. Compositions and Formulations

In certain embodiments, a subject is assigned to a treatment regimen, for example in a clinical trial, from among two or more regimens. In some embodiments, the regimens differ at least by the dose or amount of a therapeutic agent, e.g. cells that are genetically engineered with a recombinant receptor, e.g., a CAR. In particular embodiments, the regimens differ at least by the schedule of the administration of the therapeutic agent. In some embodiments, the regimens differ

In some embodiments, the cells, such as cells genetically engineered with a recombinant receptor (e.g. CAR-T cells) are provided as compositions, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions may be administered using standard administration techniques, formulations, and/or devices. Administration of the cells can be autologous or heterologous. For example, immunoresponsive cells or progenitors can be obtained from one subject, and administered to the same subject or a different, compatible subject. Peripheral blood derived immunoresponsive cells or their progeny (e.g., in vivo, ex vivo or in vitro derived) can be administered via localized injection, including catheter administration, systemic injection, localized injection, intravenous injection, or parenteral administration. When administering a therapeutic composition (e.g., a pharmaceutical composition containing a genetically modified immunoresponsive cell), it will generally be formulated in a unit dosage injectable form (solution, suspension, emulsion).

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

4. Methods of Administration

In some embodiments, the provided methods generally involve administering doses of cells expressing recombinant molecules such as recombinant receptors, such as CARs, other chimeric receptors, or other antigen receptors, such as transgenic TCRs, to subjects having a disease or condition, such as a disease or condition a component of which is specifically recognized by and/or treated by the recombinant molecules, e.g., receptors. The administrations generally effect an improvement in one or more symptoms of the disease or condition and/or treat or prevent the disease or condition or symptom thereof.

Among the diseases, conditions, and disorders are tumors, including solid tumors, hematologic malignancies, and melanomas, and including localized and metastatic tumors, infectious diseases, such as infection with a virus or other pathogen, e.g., HIV, HCV, HBV, CMV, HPV, and parasitic disease, and autoimmune and inflammatory diseases. In some embodiments, the disease or condition is a tumor, cancer, malignancy, neoplasm, or other proliferative disease or disorder. Such diseases include but are not limited to leukemia, lymphoma, e.g., chronic lymphocytic leukemia (CLL), ALL, non-Hodgkin's lymphoma, acute myeloid leukemia, multiple myeloma, refractory follicular lymphoma, mantle cell lymphoma (MCL), indolent B cell lymphoma, B cell malignancies, cancers of the colon, lung, liver, breast, prostate, ovarian, skin, melanoma, bone, and brain cancer, ovarian cancer, epithelial cancers, renal cell carcinoma, pancreatic adenocarcinoma, Hodgkin lymphoma, cervical carcinoma, colorectal cancer, glioblastoma, neuroblastoma, Ewing sarcoma, medulloblastoma, osteosarcoma, synovial sarcoma, Diffuse large B cell lymphoma (DLBCL), and/or mesothelioma.

In some embodiments, the disease or condition is a tumor and the subject has a large tumor burden prior to the administration of the first dose, such as a large solid tumor or a large number or bulk of disease-associated, e.g., tumor, cells. In some aspects, the subject has a high number of metastases and/or widespread localization of metastases. In some aspects, the tumor burden in the subject is low and the subject has few metastases. In some embodiments, the size or timing of the doses is determined by the initial disease burden in the subject. For example, whereas in some aspects the subject may be administered a relatively low number of cells in the first dose, in context of lower disease burden the dose may be higher.

In some embodiments, the disease or condition is an infectious disease or condition, such as, but not limited to, viral, retroviral, bacterial, and protozoal infections, immunodeficiency, Cytomegalovirus (CMV), Epstein-Barr virus (EBV), adenovirus, BK polyomavirus. In some embodiments, the disease or condition is an autoimmune or inflammatory disease or condition, such as arthritis, e.g., rheumatoid arthritis (RA), Type I diabetes, systemic lupus erythematosus (SLE), inflammatory bowel disease, psoriasis, scleroderma, autoimmune thyroid disease, Grave's disease, Crohn's disease, multiple sclerosis, asthma, and/or a disease or condition associated with transplant.

In some embodiments, the antigen associated with the disease or disorder is selected from the group consisting of orphan tyrosine kinase receptor ROR1, tEGFR, Her2, Ll-CAM, CD19, CD20, CD22, mesothelin, CEA, and hepatitis B surface antigen, anti-folate receptor, CD23, CD24, CD30, CD33, CD38, CD44, EGFR, EGP-2, EGP-4, 0EPHa2, ErbB2, 3, or 4, FBP, fetal acethycholine e receptor, GD2, GD3, HMW-MAA, IL-22R-alpha, IL-13R-alpha2, kdr, kappa light chain, Lewis Y, L1-cell adhesion molecule, MAGE-A1, mesothelin, MUC1, MUC16, PSCA, NKG2D Ligands, NY-ESO-1, MART-1, gp100, oncofetal antigen, ROR1, TAG72, VEGF-R2, carcinoembryonic antigen (CEA), prostate specific antigen, PSMA, Her2/neu, estrogen receptor, progesterone receptor, ephrinB2, CD123, CS-1, c-Met, GD-2, and MAGE A3, CE7, Wilms Tumor 1 (WT-1), a cyclin, such as cyclin A1 (CCNA1), and/or biotinylated molecules, and/or molecules expressed by HIV, HCV, HBV or other pathogens.

In certain embodiments, the antigen associated with the disease or disorder is selected from the group consisting of αvβ6 integrin (avb6 integrin), B cell maturation antigen (BCMA), B7-H6, carbonic anhydrase 9 (CA9, also known as CAIX or G250), a cancer-testis antigen, cancer/testis antigen 1B (CTAG, also known as NY-ESO-1 and LAGE-2), carcinoembryonic antigen (CEA), a cyclin, cyclin A2, C-C Motif Chemokine Ligand 1 (CCL-1), CD19, CD20, CD22, CD23, CD24, CD30, CD33, CD38, CD44, CD44v6, CD44v7/8, CD123, CD138, CD171, epidermal growth factor protein (EGFR), truncated epidermal growth factor protein (tEGFR), type III epidermal growth factor receptor mutation (EGFR vIII), epithelial glycoprotein 2 (EPG-2), epithelial glycoprotein 40 (EPG-40), ephrinB2, ephrine receptor A2 (EPHa2), estrogen receptor, Fc receptor like 5 (FCRLS; also known as Fc receptor homolog 5 or FCRHS), fetal acetylcholine receptor (fetal AchR), a folate binding protein (FBP), folate receptor alpha, fetal acetylcholine receptor, ganglioside GD2, O-acetylated GD2 (OGD2), ganglioside GD3, glycoprotein 100 (gp100), her2/neu (receptor tyrosine kinase erbB2), her3 (erb-B3), her4 (erb-B4), erbB dimers, human high molecular weight-melanoma-associated antigen (HMW-MAA), hepatitis B surface antigen, human leukocyte antigen A1 (HLA-AI), human leukocyte antigen A2 (HLA-A2), IL-22 receptor alpha(IL-22Ra), IL-13 receptor alpha 2 (IL-13Ra2), kinase insert domain receptor (kdr), kappa light chain, L1 cell adhesion molecule (L1CAM), CE7 epitope of L1-CAM, leucine Rich Repeat Containing 8 Family Member A (LRRC8A), Lewis Y, melanoma-associated antigen (MAGE)-A1, MAGE-A3, MAGE-A6, mesothelin, c-Met, murine cytomegalovirus (CMV), mucin 1 (MUC1), MUC16, natural killer group 2 member D (NKG2D) ligands, melan A (MART-1), neural cell adhesion molecule (NCAM), oncofetal antigen, preferentially expressed antigen of melanoma (PRAME), progesterone receptor, a prostate specific antigen, prostate stem cell antigen (PSCA), prostate specific membrane antigen (PSMA), receptor tyrosine kinase like orphan receptor 1 (ROR1), survivin, trophoblast glycoprotein (TPBG also known as 5T4), tumor-associated glycoprotein 72 (TAG72), vascular endothelial growth factor receptor (VEGFR), vascular endothelial growth factor receptor 2 (VEGFR2), Wilms tumor 1 (WT-1), a pathogen-specific antigen and an antigen associated with a universal tag.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical or similar. In some embodiments, the second subject expresses the same HLA class or supertype as the first subject.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or other agent, such as a cytotoxic or therapeutic agent. Thus, the cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents include a cytokine, such as IL-2 or other cytokine, for example, to enhance persistence.

In some embodiments, the methods comprise administration of a chemotherapeutic agent, e.g., a conditioning chemotherapeutic agent, for example, to reduce tumor burden prior to the dose administrations.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In certain embodiments, engineered cells are modified in any number of ways, such that their therapeutic or prophylactic efficacy is increased. For example, the engineered CAR or TCR expressed by the population can be conjugated either directly or indirectly through a linker to a targeting moiety. The practice of conjugating compounds, e.g., the CAR or TCR, to targeting moieties is known in the art. See, for instance, Wadwa et al., J. Drug Targeting 3: 1 1 1 (1995), and U.S. Pat. No. 5,087,616.

a. Dosing

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of no more than three days, such as once a day for three days or for two days or by multiple infusions over a single day period.

Thus, in some aspects, the cells are administered in a single pharmaceutical composition.

In some embodiments, the cells are administered in a plurality of compositions, collectively containing the cells of a single dose.

Thus, one or more of the doses in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, multiple doses are given, in some aspects using the same timing guidelines as those with respect to the timing between the first and second doses, e.g., by administering a first and multiple subsequent doses, with each subsequent dose given at a point in time that is greater than about 28 days after the administration of the first or prior dose.

In some embodiments, the dose contains a number of cells, number of recombinant receptor (e.g., CAR)-expressing cells, number of T cells, or number of peripheral blood mononuclear cells (PBMCs) in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values. In particular embodiments, the numbers and/or concentrations of cells refer to the number of recombinant receptor (e.g., CAR)-expressing cells. In other embodiments, the numbers and/or concentrations of cells refer to the number or concentration of all cells, T cells, or peripheral blood mononuclear cells (PBMCs) administered.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values. In certain embodiments, the dose comprises fewer than about 1×10¹², about 1×10¹¹, about 1×10¹⁰, about 1×10⁹, about 1×10⁸, or about 1×10⁷, total recombinant receptor (e.g., CAR)-expressing cells. In particular embodiments, the dose includes between about 1×10⁶ to 1×10⁸, about 1×10⁹ to 1×10¹¹, about 1×10¹⁰ to 1×10¹² total recombinant receptor (e.g., CAR)-expressing cells, T cells, or peripheral blood mononuclear cells (PBMCs). In certain embodiments, the dose includes between about 1×10⁶ to 1×10⁷′ about 1×10⁹ to 1×10¹¹ such cells, about 1×10¹⁰ to 1×10¹² total recombinant receptor (e.g., CAR)-expressing cells, T cells, or peripheral blood mononuclear cells (PBMCs).

In some embodiments, the dose contains fewer than about 1×10⁸ total recombinant receptor (e.g., CAR)-expressing cells, T cells, or peripheral blood mononuclear cells (PBMCs) cells per m² of the subject, e.g., in the range of about 1×10⁶ to 1×10⁸ such cells per m² of the subject, such as 2×10⁶, 5×10⁶, 1×10⁷, 5×10⁷, or 1×10⁸ such cells per m² of the subject, or the range between any two of the foregoing values.

In certain embodiments, the number of cells, recombinant receptor (e.g., CAR)-expressing cells, T cells, or peripheral blood mononuclear cells (PBMCs) in the dose is greater than about 1×10⁶ such cells per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, 1×10⁸, or 1×10⁹, 1×10¹⁰ such cells per m² of the subject or total, or the range between any two of the foregoing values.

In some embodiments, the dose is determined based on total number of CD4+ CAR+ T cells and/or CD8+ CAR+ T-cells. In some embodiments, the dose is determined based on a ratio of CD4+ CAR+ T cells:CD8+ CAR+ T cells. In some embodiments, the dose is determined based on total number of CD4+ and/or CD8+ T cells. In some embodiments, the dose is determined based on a ratio of CD4+:CD8+ T cells. In some embodiments, the dose is a fixed and/or flat dose. In some embodiments, the dose is dependent on a physical parameter of a subject, e.g. weight in kg or m2.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

In some aspects, the size of the dose is determined by the burden of the disease or condition in the subject. For example, in some aspects, the number of cells administered in the dose is determined based on the tumor burden that is present in the subject immediately prior to administration of the initiation of the dose of cells. In some embodiments, the size of the first and/or subsequent dose is inversely correlated with disease burden. In some aspects, as in the context of a large disease burden, the subject is administered a low number of cells, for example less than about 1×10⁶ cells per kilogram of body weight of the subject. In other embodiments, as in the context of a lower disease burden, the subject is administered a larger number of cells, such as more than about 1×10⁶ cells per kilogram body weight of the subject.

### V. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, a “subject” is a mammal, such as a human or other animal, and typically is human.

### VI. EXEMPLARY EMBODIMENTS

Among the provided embodiments are:

1. A method for adaptive allocation of a subject to a treatment regimen for administering a therapeutic agent in a clinical trial, comprising:
a) designating two or more possible unique treatment regimens for administering a therapeutic agent to a population of subjects enrolled in a clinical trial, which subjects comprise two or more diseases, wherein each of the treatment regimens differ in one or both of a dose level and a schedule;
b) calculating an overall disease-specific utility score for each disease cohort for each treatment regimen, wherein the overall disease-specific utility score is based on response information of subjects within each disease cohort previously treated with the therapeutic agent according to each treatment regimen and on toxicity information of subjects across all disease cohorts previously treated with the therapeutic agent according to each treatment regimen; and
c) allocating the subject to a treatment regimen based on the overall disease cohort utility score.

2. The method of embodiment 1, wherein allocating the subjects to a treatment regimen is further based on the number of subjects already allocated to one or more of the treatment regimens or the number of open spots in each treatment regimen.

3. The method of embodiment 1 or embodiment 2, wherein after allocating the subject to the regimen, the method further comprises administering to the subject the therapeutic agent according to the treatment regimen in which the subject has been allocated.

4. A method for adaptive treatment of a subject in a clinical trial, comprising:
a) designating two or more possible unique treatment regimens for administering a therapeutic agent to a population of subjects enrolled in a clinical trial, which subjects comprise two or more diseases, wherein each of the treatment regimens differ in one or both of a dose level and a schedule;
b) calculating an overall disease-specific utility score for each disease cohort for each treatment regimen, wherein the overall disease-specific utility score is based on response information of subjects within each disease cohort previously treated with the therapeutic agent according to each treatment regimen and on toxicity information of subjects across all disease cohorts previously treated with the therapeutic agent according to each treatment regimen;
c) allocating a subject to a treatment regimen based on the overall disease cohort utility score; and
d) administering to the subject the therapeutic agent according to the treatment regimen in which the subject has been allocated.

5. The method of embodiment 4, wherein allocating the subjects to a treatment regimen is further based on the number of subjects already allocated to one or more of the treatment regimens or the number of open spots in each treatment regimen.

6. The method of any of embodiments 1-5, wherein at least two of the two or more possible unique regimens differ in the dose level and the schedule.

7. The method of any of embodiments 1-6, wherein the two or more diseases comprise two or more disease subtypes.

8. The method of any of embodiments 1-7, wherein calculating the overall disease-specific utility score for each disease cohort for each treatment regimen comprises:
i) determining a toxicity rate based on the toxicity information of subjects previously treated across all disease cohorts for each treatment regimen;
ii) determining an efficacy rate based on the response information of subjects previously treated within each disease cohort for each treatment regimen;
iii) generating a safety utility score as a function of the toxicity rate for the treatment regimen;
iv) generating an efficacy utility score as a function of the efficacy rate for the treatment regimen; and
v) multiplying the safety utility score by the efficacy utility score, thereby calculating the overall disease-specific utility score for each disease cohort for each treatment regimen.

9. The method of embodiment 8, wherein the method further comprises reporting the overall disease-specific utility score.

10. The method of embodiment 8 or embodiment 9, wherein in step iii):
if the toxicity rate is determined to be less than or equal to a utility safety target, then the function defines the safety utility score as 1;
if the toxicity rate is determined to be greater or equal to a utility safety limit, then the function defines the safety utility score as 0; and
if the toxicity rate is projected to be between the utility safety target and the utility safety limit, then the safety utility score decreases linearly as the toxicity rate increases.

11. The method of any of embodiments 8-10, wherein in step iv):
if the efficacy rate is projected to be less than or equal to a utility efficacy target, then the function defines the efficacy utility score as 0;
if the efficacy rate is projected to be greater than the utility efficacy target, then the efficacy utility score increases linearly as the efficacy rate increases.

12. The method of any of embodiments 8-11, wherein the toxicity rate is a dose-limiting toxicity (DLT) rate.

13. The method of any of embodiments 8-12, wherein the response rate is a complete response (CR) rate.

14. The method of any of embodiments 8-13, wherein the toxicity rate is determined by a regimen-toxicity model that estimates a single toxicity rate for each regimen across all disease cohorts.

15. The method of embodiment 14, where the regimen-toxicity model comprises the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {β}_{3} {1}_{[ s = 2 ⋂ d = - 1 ]}$$

16. The method of any of embodiments 8-15, wherein the efficacy rate is determined by a regimen-response model that estimates a single efficacy rate within each disease cohort for each regimen.

17. The method of embodiment 16, wherein the regimen-response model borrows response information across disease cohorts.

18. The method of embodiment 16 or embodiment 17, wherein the regimen response model comprises the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

19. The method according to embodiment 17 or embodiment 18, wherein the response information is borrowed across disease cohorts according to the following distribution:

$${α}_{0 , DLBCL} , {α}_{0 , MCL} \sim N ( θ , {σ}^{2} ) ; θ \sim N ( - 0.5 , {4}^{2} ) ; \frac{1}{{σ}^{2}} \sim Gamma ( 2 , 2 ) .$$

20. The method of any of embodiments 1-19, wherein:
each of the two or more treatment regimens has a first status of either open or closed; and
the subject is allocated into an open treatment regimen.

21. The method of embodiment 20, wherein prior to allocating the subject, the method further comprises:
determining if the first status of any of the treatment regimens should be changed from closed to open; and
if the first status of a treatment regimen should be changed, changing the first status to open.

21. The method of embodiment 20, wherein determining if the first status of a treatment regimen should be changed comprises determining if another open regimen is considered to be safe, effective, unsafe, or ineffective.

22. The method of embodiment 21, wherein:
a treatment regimen is determined to be unsafe for the purpose of opening another regimen probability that the toxicity rate is less than a first open safety limit is less than an open toxicity probability parameter; and/or
a treatment regimen is determined to be ineffective for the purpose of opening another regimen the probability that the efficacy rate is greater than a first open efficacy target is less than an open efficacy probability parameter.

23. The method of embodiment 22, wherein:
the utility and first open safety limits can be the same or different; and/or
the first and second efficacy targets can be the same or different.

24. The method of any of embodiments 21-23, wherein:
a treatment regimen is determined to be safe for the purpose of opening another regimen if the probability that the toxicity rate is less than a second safety limit is greater than a second toxicity probability parameter; and/or
a treatment regimen is determined to be effective for the purpose of opening another regimen if the probability that the efficacy rate is greater than a second efficacy target is greater than a second efficacy probability parameter.

25. The method of embodiment 24, wherein:
any of the utility, first, and second safety targets can be the same or different; and/or
any of the utility, first, and second, efficacy targets can be the same or different.

26. The method of any of embodiments 21-25, wherein the status of a first regimen is open, and wherein a second regimen is a de-escalation regimen of the first open regimen.

27. The method of embodiment 26, wherein the status of the second regimen is changed to open when the first regimen is determined to be unsafe for the purpose of opening another regimen.

28. The method of any of embodiments 21-27, wherein the status of the first regimen is open, and wherein a third regimen is an escalation regimen of the first open regimen.

29. The method of embodiment 28, wherein the status of the third regimen is changed to open when the first regimen is determined to be ineffective for the purpose of opening another regimen.

30. The method of embodiment 28 or embodiment 29, wherein the status of the third regimen is changed to open when the first regimen is determined to be safe for the purpose of opening another regimen.

31. The method of any of embodiments 21-30, wherein the status of the second regimen is open and the status of the third regimen is open, and wherein a fourth regimen is an escalation of the second regimen and a de-escalation of the third regimen.

32. The method of embodiment 31, wherein the status of the fourth regimen is changed to open when:
the first regimen is determined to be safe for the purpose of opening another regimen and the third regimen is determined to be unsafe for the purpose of opening another regimen; or
the second regimen is determined to be safe for the purpose of opening another regimen and the fourth regimen is determined to be safe for the purpose of opening another regimen across disease cohorts.

33. The method of any of embodiments 20-32, wherein regimen with a first status of open comprises a second status, wherein the second status can be either eligible or suspended.

34. The method of embodiment 33, wherein the second status is assigned to be eligible when the first status is changed to open.

35. The method of embodiment 33 or embodiment 34, wherein the second status is temporarily changed to suspended:
if the probability that the toxicity rate is less than a third safety limit is less than a third toxicity probability parameter; and/or
if the probability that the efficacy rate is greater than a third efficacy target is less than a third efficacy probability parameter.

36. The method of embodiment 35, wherein
any of the utility, first, second, and third safety targets can be the same or different; and/or
any of the utility, first, second, and third efficacy targets can be the same or different.

37. The method of any of embodiments 34-36, wherein:
a treatment regimen is determined to be safe for the purpose of suspending the regimen if the probability that the toxicity rate is less than a fourth safety limit is greater than a fourth toxicity probability parameter; and/or
the treatment regimen is determined to be effective for the purpose of suspending the regimen if the probability that the efficacy rate is greater than a fourth efficacy target is greater than a fourth efficacy probability parameter.

38. The method of embodiment 37, wherein:
any of the utility, first, second, third, and fourth safety targets can be the same or different; and/or
any of the utility, first, second, third, and fourth efficacy targets can be the same or different.

39. The method of any of embodiments 1-38, wherein allocating the subject to a treatment regimen is further based on the relative uncertainty of the estimate for the utility of each regimen.

40. The method of embodiment 39, wherein allocating a subject to a treatment regimen comprises a randomization probability that a subject will be enrolled in each of the regimen within each disease cohort with a first status of open and a second status of eligible, wherein the randomization probability V_{d,s,h} is:

$${V}_{d , s , h} ∝ {[ \frac{Pr ( {r}_{d , s , h} = {r}_{{d}^{*} , {s}^{*} , h} ) Var ( {U}_{d , s , h} )}{{n}_{d , s , h} + 1} ]}^{\frac{1}{2}} .$$

wherein r_{d,s,h} is the regimen of dose level d, schedule s, and disease h, Pr(r_{d,s,h}=r_{d*,s*,h}) is the probability the regimen is the highest utility regimen, Var(U_{d,s,h}) is the variance of the regimen's disease-specific utility score, and n_{d,s,h} is the number of subjects already allocated to the regimen within disease cohort h.

41. The method of any of embodiments 1-40, wherein the clinical trial ends if:
none of the regimens have a first status of open and a second status of eligible; or
a total number of enrolled subjects is equal to or greater than a maximum subject enrollment limit;

42. The method of any of embodiments 1-41, wherein the clinical trial ends for one of the two or more diseases if:
none of the regimens for the disease have a first status of open and a second status of eligible;
a total number of enrolled subjects with the disease is equal to or greater than a maximum disease-subject enrollment limit;
response and toxicity information within the disease cohort has been determined for a first pre-determined minimum number of subjects within the regimen with the highest utility and the regimen is unlikely to be effective within the cohort for the purpose of termination; or
response and toxicity information within the disease cohort has been determined for a second pre-determined minimum number of subjects and the regimen with the highest utility is highly likely to be safe and effective within the cohort for the purpose of termination.

43. The method of embodiment 42, wherein a regimen is unlikely to be effective within the cohort for the purpose of termination if the probability that the efficacy rate is greater than a termination efficacy target is less than a termination efficacy probability parameter.

44. The method of embodiment 42 or 43, wherein a regimen is unlikely to be safe across the cohort for the purpose of termination if the probability that the toxicity rate is less than a termination safety limit is less than a termination toxicity probability parameter.

45. The method of any of embodiments 42-44, wherein a regimen is likely to be safe and effective within the cohort for the purpose of termination for early success if:
the probability that the toxicity rate is less than a success safety limit is greater than a success toxicity probability parameter; and
the probability that the efficacy rate is greater than a success efficacy target is greater than a success efficacy probability parameter.

46. The method of any of embodiments 1-45, wherein the therapeutic agent is one in which the response information can be determined within the same period of time in which the toxicity information is determined.

47. The method of any of embodiments 1-46, wherein the therapeutic agent comprises an adoptive cell therapy, a small molecule, a gene therapy, or a transplant.

48. The method of any of embodiments 3-47, wherein the therapeutic agent comprises cells expressing a chimeric antigen receptor (CAR).

49. The method of embodiments 48, wherein the CAR expressed by the cells specifically binds to an antigen expressed by a cell or tissue of at least one of the two or more diseases or associated with at least one of the two or more diseases.

50. The method of any of embodiments 1-49, wherein at least one of the two or more diseases is a tumor or a cancer.

51. The method of any of embodiments 47-50, wherein the number of cells administered in the first dose is between about 0.5×10⁶ cells/kg body weight of the subject and 6×10⁶ cells/kg, between about 0.75×10⁶ cells/kg and 2.5×10⁶ cells/kg, between about 1×10⁶ cells/kg and 2×10⁶ cells/kg, between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive.

52. The method of any of embodiments 47-51, wherein the dose of cells are administered in a single pharmaceutical composition comprising the cells of the dose.

53. The method of any of embodiments 47-52, wherein:
the dose is a split dose, wherein the cells of the dose are administered in a plurality of compositions, collectively comprising the cells of the dose, over a period of no more than three days.

54. The method of any of embodiments 49-53, wherein at least one of the two or more diseases is a leukemia or lymphoma.

55. The method of any of embodiments 49-54, wherein at least one of the two or more diseases is acute lymphoblastic leukemia.

56. The method of any of embodiments 1-55, wherein at least one of the two or more diseases is a non-Hodgkin lymphoma (NHL).

57. The method of embodiment 56, wherein at least one of the two or more diseases is diffuse large B cell lymphoma (DLBCL).

58. The method of embodiment 56 or embodiment 57, wherein at least one of the two or more diseases is mantle cell lymphoma (MCL).

59. The method of any of embodiments 1-58 that is repeated for one or more additional subject enrolled in the clinical trial.

60. The method of any of embodiments 1-59, wherein the clinical trial is a phase I clinical trial.

61. A method of calculating an overall disease-specific utility score for each disease cohort for each treatment regimen in a clinical trial, comprising:
a) determining a toxicity rate based on the toxicity information of subjects previously treated across all disease cohorts for each treatment regimen;
b) determining an efficacy rate based on the response information of subjects previously treated within each disease cohort for each treatment regimen;
c) generating a safety utility score as a function of the toxicity rate for the treatment regimen;
d) generating an efficacy utility score as a function of the efficacy rate for the treatment regimen; and
e) multiplying the safety utility score by the efficacy utility score, thereby calculating the overall disease-specific utility score for each disease cohort for each treatment regimen.

62. The method of embodiment 61, wherein the method further comprises reporting the overall disease-specific utility score for each disease cohort for each treatment regimen.

63. The method of embodiment 61 or embodiment 62, wherein in step c):
if the toxicity rate is determined to be less than or equal to a first safety target, then the function defines the safety utility score as 1;
if the toxicity rate is determined to be greater or equal to a first safety limit, then the function defines the safety utility score as 0; and
if the toxicity rate is projected to be between the first safety target and the first safety limit, then the safety utility score decreases linearly as the toxicity rate increases.

64. The method of any of embodiments 61-63, wherein in step d):
if the efficacy rate is projected to be less than or equal to an efficacy target, then the function defines the efficacy utility score as 0;
if the efficacy rate is projected to be greater than the efficacy target, then the efficacy utility score increases linearly as the efficacy rate increases.

65. The method of any of embodiments 61-64, wherein the toxicity rate is a dose-limiting toxicity (DLT) rate.

66. The method of any of embodiments 61-65, wherein the response rate is a complete response (CR) rate.

67. The method of any of embodiments 61-66, wherein the toxicity rate is determined by a regimen-toxicity model that a single toxicity rate for each regimen across all disease cohorts.

68. The method of embodiment 67, where the regimen-toxicity model comprises the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {β}_{3} {1}_{[ s = 2 ⋂ d = - 1 ]}$$

69. The method of any of embodiments 61-68, wherein the efficacy rate is determined by a regimen-response model that estimates a single efficacy rate within each disease cohort for each regimen.

70. The method of embodiment 69, wherein the regimen-response model borrows response information across disease cohorts.

71. The method of embodiment 69 or embodiment 70, wherein the regimen response model comprises the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

72. The method according to embodiment 70 or embodiment 71, wherein the response information is borrowed across disease cohorts according to the following distribution:

$${α}_{0 , DLBCL} , {α}_{0 , MCL} \sim N ( θ , {σ}^{2} ) ; θ \sim N ( - 0.5 , {4}^{2} ) ; \frac{1}{{σ}^{2}} \sim Gamma ( 2 , 2 ) .$$

73. The method of any of embodiments 1-72, wherein the subject is a human subject.

74. The method of any of embodiments 1-73, wherein the method is a computer implemented method, and wherein one or more steps a)-c) occur at an electronic device comprising one or more processors and memory.

75. A computer system comprising a processor and memory, the memory comprising instructions operable to cause the processor to carry out any one or more of steps a)-c) of the methods of any of embodiments 1-60.

76. The method of any of embodiments 61-73, wherein the method is a computer implemented method, and wherein one or more steps occur at an electronic device comprising one or more processors and memory.

77. A computer system comprising a processor and memory, the memory comprising instructions operable to cause the processor to carry out any one or more of steps of the methods of any of embodiments 61-73 or 76.

Also among the provided embodiments are:

1. A method for allocating a subject to a selected treatment regimen for administering a therapeutic agent, comprising:
a) designating two or more possible unique treatment regimens for administering a therapeutic agent to a population of subjects;
b) calculating an overall utility score for at least one treatment regimen, wherein, for each of said at least one treatment regimen, the overall utility score is based on response information of, and on toxicity information of, one or more subjects previously treated with the therapeutic agent according to the treatment regimen; and
c) allocating the subject to a selected treatment regimen based on the overall utility score.

2. A method for allocating a subject to a selected treatment regimen for administering a therapeutic agent, comprising:

a) calculating an overall utility score for at least one of a plurality of unique treatment regimens for administering a therapeutic agent, wherein, for each of the at least one treatment regimens, the overall utility score is based on response information of, and on toxicity information of, one or more subjects previously treated with the therapeutic agent according to the treatment regimen; and

b) allocating the subject to a selected treatment regimen based on the overall utility score.

3. A method for allocating a subject to a selected treatment regimen for administering a therapeutic agent, the method comprising:

allocating the subject to a selected treatment regimen based on an overall utility score for at least one treatment regimen, wherein the overall utility score is based on response information of, and on toxicity information of, one or more subjects previously treated with the therapeutic agent according to the treatment regimen.

4. The method of any of embodiments 1-3, further comprising treating the subject with the selected treatment regimen.

5. The method of any of embodiments 1-3, wherein the selected treatment regimen is one of the at least one treatment regimen.

6. The method of any of embodiments 1-3, wherein the selected treatment regimen is not one of the at least one treatment regimen.

7. A method for treatment of a subject with a therapeutic agent, comprising:
a) designating two or more possible unique treatment regimens for administering a therapeutic agent;
b) calculating an overall utility score for at least one treatment regimen, wherein the overall utility score is based on response information of subjects and on toxicity information of subjects previously treated with the therapeutic agent according to the at least one treatment regimen;
c) allocating a subject to a selected treatment regimen based on the overall utility score; and
d) administering to the subject the therapeutic agent according to the selected treatment regimen in which the subject has been allocated.

8. A method for treatment of a subject with a therapeutic agent, comprising administering to a subject a therapeutic agent according to a selected treatment regimen,
wherein the selected treatment regimen is selected from two or more possible unique treatment regimens for administering the therapeutic agent, based on an overall utility score that is calculated for at least one treatment regimen, wherein the overall utility score for each of the at least one treatment regimen is based on response information of subjects of, and on toxicity information of, one or more subjects previously treated with the therapeutic agent according to the treatment regimen.

9. The method of any of embodiments 1-8, wherein the calculating of the overall utility score for at least one treatment regimen comprises calculating the overall utility score for each of a plurality of different treatment regimens for administering the therapeutic agent.

10. The method of any of embodiments 1-9, wherein the one or more subjects are enrolled in a clinical trial and/or wherein the previous treatment of the one or more subjects is carried out in a single clinical trial and/or the subject to whom the selected treatment regimen is allocated is enrolled in a clinical trial and/or wherein the subject to whom the selected treatment regimen is allocated and the one or more subjects are enrolled in the same clinical trial.

11. The method of embodiment 10, wherein the clinical trial comprises an open enrollment design.

12. The method of any of embodiments 1-11, wherein the allocation of the subject comprises random allocation of the subject to the selected treatment regimen based on a randomization probability, wherein a regimen with a higher utility score has a greater randomization probability than a regimen with lower utility score and/or where height of utility score positively influences randomization probability.

13. The method of any of embodiments 1-12, wherein allocating the subject to the selected treatment regimen is further based on the number of subjects already allocated to one or more of the treatment regimens or the number of open spots in each treatment regimen

14. The method of embodiment 13, wherein a trial has an open spot if less than three subjects with unknown toxicity information are currently enrolled in the regimen, or if at least six subjects with complete toxicity information have been enrolled in the regimen and less than four subjects with unknown toxicity are enrolled in the regimen.

15. The method of any of embodiments 1-14, wherein the one or more subjects comprises at least one subject in each of two or more disease cohorts.

16. The method of any of embodiments 1-15, wherein the calculating the overall utility score for at least one treatment regimen comprises calculating the overall utility score for the at least one treatment regimen comprises calculating a separate utility score for the treatment regimen for each of a plurality of disease cohorts.

17. The method of embodiment 15 or 16, wherein each of said two or more disease cohorts comprises subjects with one of a plurality of different diseases, one of a plurality of different grades of disease, one of a plurality of disease burden levels, one of a plurality of states of genetic mutation associated with the disease or condition and/or the treatment regimen, one of a plurality of tumor locations, one of a plurality of age groups of subjects with a disease, one of a gender of subjects with a disease, one of a plurality of weight groups of subjects with a disease, and/or one of a plurality of different number and/or types of prior therapies or treatments for the disease.

18. The method of any of embodiments 15-17, wherein subjects with different subtypes of the same disease are grouped into different disease cohorts.

19. The method of any of embodiments 15-18, wherein said calculating said utility score for said at least one treatment regimen comprises, for each of said at least one treatment regimen, individually, calculating an overall disease-specific utility score for each of a plurality of disease cohorts for said treatment regimen, wherein the overall disease-specific utility score is based on response information of subjects within the disease cohort treated with the therapeutic agent according to the treatment regimen and on toxicity information for subjects across a plurality of disease cohorts treated with the therapeutic agent according to the treatment regimen.

20. The method of any of embodiments 1-19, wherein said at least one treatment regimen comprises two or more unique treatment regimens, wherein at least two of the two or more unique treatment regimens differ in dose level of the therapeutic agent.

21. The method of any of embodiments 1-20, wherein said at least one treatment regimen comprises two or more unique treatment regimens, wherein at least two of the two or more possible unique regimens differ by a schedule of administration of the therapeutic agent.

22. The method of any of embodiments 1-21, wherein said at least one treatment regimen comprises two or more unique treatment regimens, wherein at least two of the two or more possible unique regimens differ by dose level and by schedule.

23. The method of any of embodiments 15-22, wherein calculating the overall disease-specific utility score for each treatment regimen for a given disease cohort comprises:
i) determining a toxicity rate based on the toxicity information of a population of subjects treated according to the treatment regimen;
ii) determining an efficacy rate based on response information of a population of subjects treated according to the treatment regimen;
iii) generating a safety utility score based on a function of the toxicity rate;
iv) generating an efficacy utility score based on a function of the efficacy rate; and
v) multiplying the safety utility score for the treatment regimen by the efficacy utility score, thereby calculating the overall disease-specific utility score for the treatment regimen for the disease cohort.

24. The method of embodiment 23, wherein:
the population of subjects from which the toxicity information is determined comprises subjects in the disease cohort and subjects not in the disease cohort; and/or
the population of subjects from which the efficacy rate is determined comprises subjects in the disease cohort and does not comprise subjects not in the disease cohort.

25. The method of any of embodiments 15-22, wherein calculating the overall disease-specific utility score for each treatment regimen for a given disease cohort comprises:

i) generating a safety utility score, wherein said safety utility score is based on a toxicity rate for the treatment regimen;

ii) generating an efficacy utility score for the treatment regimen, wherein said efficacy utility score is based on an efficacy rate for the treatment regimen; and

iii) multiplying the safety utility score by the efficacy utility score, thereby calculating the overall disease-specific utility score for the treatment regimen for the disease cohort.

26. The method of embodiment 25, wherein:
the safety utility score is based on toxicity information from subjects in the disease cohort and from subjects not in the disease cohort; and/or
the efficacy utility score is based on efficacy information from subjects in the disease cohort and not for subjects not in the disease cohort.

27. The method of any of embodiments 15-22, wherein calculating the overall disease-specific utility score for the treatment regimen for the disease cohort comprises multiplying a safety utility score for the treatment regimen by an efficacy utility score for the treatment regimen, wherein the safety utility score is based on a toxicity rate for the treatment regimen among subjects within the disease cohort and subjects not within the disease cohort and the efficacy utility score is based on an efficacy rate for the treatment regimen for subjects within the disease cohort and not subjects not within the disease cohort.

28. The method of any of embodiments 15-22, wherein calculating the overall disease-specific utility score for each disease cohort for each treatment regimen comprises:
i) determining a toxicity rate based on the toxicity information of a population of subjects treated according to the treatment regimen;
ii) determining an efficacy rate based on response information of a population of subjects treated according to the treatment regimen;
iii) generating a safety utility score as a function of the toxicity rate for the treatment regimen;
iv) generating an efficacy utility score as a function of the efficacy rate for the treatment regimen; and
v) multiplying the safety utility score by the efficacy utility score, thereby calculating the overall disease-specific utility score for each disease cohort for each treatment regimen.

29. The method of any of embodiments 1-28, wherein the method further comprises reporting the overall disease-specific utility score.

30. The method of any of embodiments 23-29, wherein in step iii):
if the toxicity rate is determined to be less than or equal to a utility safety target, then the function defines the safety utility score as 1;
if the toxicity rate is determined to be greater or equal to a utility safety limit, then the function defines the safety utility score as 0; and
if the toxicity rate is projected to be between the utility safety target and the utility safety limit, then the safety utility score decreases linearly as the toxicity rate increases.

31. The method of embodiment 30, wherein the safety utility score is set as zero when the toxicity rate is above a 0.33, and the safety utility score is set at zero when the toxicity rate is below a 0.2, and wherein the safety utility score decreases in a linear manner from one to zero when the toxicity rate increases from 0.20 to 0.33.

32. The method of any of embodiments 23-29, wherein in step iv):
if the efficacy rate is projected to be less than or equal to a utility efficacy target, then the function defines the efficacy utility score as 0;
if the efficacy rate is projected to be greater than the utility efficacy target, then the efficacy utility score increases linearly as the efficacy rate increases.

33. The method of embodiment 32, wherein the efficacy utility score is set as zero when the efficacy rate is below 0.25, and the efficacy utility score is set at one when the efficacy rate is 1, and wherein the efficacy utility score increases in a linear manner from zero to one when the efficacy rate increases from 0.25 to 1.

34. The method of any of embodiments 28-33, wherein the toxicity rate is a dose-limiting toxicity (DLT) rate.

35. The method of any of embodiments 28-34, wherein the efficacy rate is a complete response (CR) rate.

36. The method of any of embodiments 28-35, wherein the toxicity rate is determined by a regimen-toxicity model that estimates a single toxicity rate for each regimen across all disease cohorts.

37. The method of embodiment 36, wherein the toxicity rate is determined by a formula, whereby the formula is a logit function, wherein a first regimen is set as the referent, and wherein the logit function comprises the sum of the additive effects of the first regimen and of each additional regimen as compared to the first regimen.

38. The method of embodiment 37, wherein the additive effect of a regimen is constrained to be negative if the regimen comprises administering less of a total amount of the therapeutic agent than the first regimen, and wherein the additive effect of a regimen is constrained to be positive if the regimen comprises administering more of a total amount of the therapeutic agent than the first regimen.

39. The method of any of embodiments 37-39, wherein the regimen-toxicity model comprises the formula:

$$log ( \frac{π}{1 - π} ) = {β}_{0} + {β}_{1} {X}_{1} … + {β}_{n} {X}_{n}$$

wherein π is the DLT rate, wherein β₀ is a first regimen that is set as the referent, wherein β₁ X₁ . . . βₙXₙ are the additive effects of the remaining regimens in relation to the first regimen, and wherein is n is equal to the number of regimens minus one.

40. The method of embodiment 39, where the regimen-toxicity model comprises the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {β}_{3} {1}_{[ s = 2 ⋂ d = - 1 ]}$$

41. The method of any of embodiments 28-40, wherein the efficacy rate is determined by a regimen-response model that estimates a single efficacy rate within each disease cohort for each regimen.

42. The method of embodiment 41, wherein the regimen-response model borrows response information across disease cohorts.

43. The method of embodiment 41 or 42, wherein the efficacy rate is determined by a formula, whereby the formula is a logit function, wherein a first regimen is set as the referent, and wherein the logit function comprises the sum of the additive effects of the first regimen and of each additional regimen as compared to the first regimen.

44. The method of any of embodiments 41-43, wherein the additive effect of a regimen is constrained to be negative if the regimen comprises administering less of a total amount of the therapeutic agent than the first regimen, and wherein the additive effect of a regimen is constrained to be positive if the regimen comprises administering more of a total amount of the therapeutic agent than the first regimen.

45. The method of any of embodiments 41-44, wherein the regimen-efficacy model comprises the formula:

$$log ( \frac{θ}{1 - θ} ) = {α}_{0} + {α}_{1} {X}_{1} … + {α}_{n} {X}_{n}$$

wherein θ is the CR rate, wherein α₀ is a first regimen that is set as the referent, wherein α₁ X₁ . . . αₙXₙ are the additive effects of the remaining regimens in relation to the first regimen, and wherein is n is equal to the number of regimens minus one.

46. The method of any of embodiments 41-45, wherein the regimen response model comprises the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

47. The method according to any of embodiments 41-46, wherein the response information is borrowed across disease cohorts according to the following distribution:

$${α}_{0 cohort 1} , {α}_{0 cohort \hat{2}} \cdot N ( θ , {σ}^{2} ) ; θ \sim N ( - 0.5 , {4}^{2} ) ; \frac{1}{{σ}^{2}} \sim Gamma ( 2 , 2 ) .$$

48. The method of any of embodiments 1-47, wherein:
each of the two or more treatment regimens has a first status of either open or closed; and
the subject is allocated into an open treatment regimen.

49. The method of embodiment 48, wherein prior to allocating the subject, the method further comprises:
determining if the first status of any of the treatment regimens should be changed from closed to open; and
if the first status of a treatment regimen should be changed, changing the first status to open.

50. The method of embodiment 49, wherein determining if the first status of a treatment regimen should be changed comprises determining if another open regimen is considered to be safe, effective, unsafe, or ineffective.

51. The method of embodiment 50, wherein:
a treatment regimen is determined to be unsafe for the purpose of opening another regimen when the probability that the toxicity rate of said another regimen is less than a first open safety limit is less than an open toxicity probability parameter; and/or
a treatment regimen is determined to be ineffective for the purpose of opening another regimen the probability that the efficacy rate of said treatment regimen is greater than a first open efficacy target is less than an open efficacy probability parameter.

52. The method of embodiment 51, wherein:
the utility and first open safety limits can be the same or different; and/or
the first and second efficacy targets can be the same or different.

53. The method of any of embodiments 50-52, wherein:
a treatment regimen is determined to be safe for the purpose of opening another regimen if the probability that the toxicity rate is less than a second safety limit is greater than a second toxicity probability parameter; and/or
a treatment regimen is determined to be effective for the purpose of opening another regimen if the probability that the efficacy rate is greater than a second efficacy target is greater than a second efficacy probability parameter.

54. The method of embodiment 53, wherein:
any of the utility, first, and second safety targets can be the same or different; and/or
any of the utility, first, and second, efficacy targets can be the same or different.

55. The method of any of embodiments 50-54, wherein the status of a first regimen is open, and wherein a second regimen is a de-escalation regimen of the first open regimen.

56. The method of embodiment 55, wherein the status of the second regimen is changed to open when the first regimen is determined to be unsafe for the purpose of opening another regimen.

57. The method of any of embodiments 50-56, wherein the status of the first regimen is open, and wherein a third regimen is an escalation regimen of the first open regimen.

58. The method of embodiment 57, wherein the status of the third regimen is changed to open when the first regimen is determined to be ineffective for the purpose of opening another regimen.

59. The method of embodiment 57 or embodiment 58, wherein the status of the third regimen is changed to open when the first regimen is determined to be safe for the purpose of opening another regimen.

60. The method of any of embodiments 50-59, wherein the status of the second regimen is open and the status of the third regimen is open, and wherein a fourth regimen is an escalation of the second regimen and a de-escalation of the third regimen.

61. The method of embodiment 60, wherein the status of the fourth regimen is changed to open when:
the first regimen is determined to be safe for the purpose of opening another regimen and the third regimen is determined to be unsafe for the purpose of opening another regimen; or
the second regimen is determined to be safe for the purpose of opening another regimen and the fourth regimen is determined to be safe for the purpose of opening another regimen across disease cohorts.

62. The method of any of embodiments 49-61, wherein regimen with a first status of open comprises a second status, wherein the second status can be either eligible or suspended.

63. The method of embodiment 62, wherein the second status is assigned to be eligible when the first status is changed to open.

64. The method of embodiment 62 or embodiment 63, wherein the second status is temporarily changed to suspended:
if the probability that the toxicity rate is less than a third safety limit is less than a third toxicity probability parameter; and/or
if the probability that the efficacy rate is greater than a third efficacy target is less than a third efficacy probability parameter.

65. The method of embodiment 64, wherein
any of the utility, first, second, and third safety targets can be the same or different; and/or
any of the utility, first, second, and third efficacy targets can be the same or different.

66. The method of any of embodiments 63-65, wherein:
a treatment regimen is determined to be safe for the purpose of suspending the regimen if the probability that the toxicity rate is less than a fourth safety limit is greater than a fourth toxicity probability parameter; and/or
the treatment regimen is determined to be effective for the purpose of suspending the regimen if the probability that the efficacy rate is greater than a fourth efficacy target is greater than a fourth efficacy probability parameter.

67. The method of embodiment 66, wherein:
any of the utility, first, second, third, and fourth safety targets can be the same or different; and/or
any of the utility, first, second, third, and fourth efficacy targets can be the same or different.

68. The method of any of embodiments 1-67, wherein allocating the subject to a selected treatment regimen is further based on the relative uncertainty of the estimate for the utility of each regimen.

69. The method of embodiment 68, wherein allocating a subject to a selected treatment regimen is based on a randomization probability that a subject will be enrolled in each of the regimens with a first status of open and a second status of eligible, wherein the randomization probability Vₓ is:

$${V}_{x} ∝ {[ \frac{Pr ( {r}_{x} = {r}_{{x}^{*}} ) Var ( {U}_{x} )}{{n}_{x} + 1} ]}^{\frac{1}{2}} .$$

wherein rₓ is the regimen x, Pr(rₓ=r_{x*}) is the probability the regimen x is the highest utility regimen, Var(Uₓ) is the variance of the regimen's utility score, and nₓ is the number of subjects already allocated to the regimen.

70. The method of embodiment 68, wherein allocating a subject to a selected treatment regimen is based on a randomization probability that a subject will be enrolled in each of the regimen within each disease cohort with a first status of open and a second status of eligible, wherein the randomization probability V_{d,s,h} is:

$${V}_{d , s , h} ∝ {[ \frac{Pr ( {r}_{d , s , h} = {r}_{{d}^{*} , {s}^{*} , h} ) Var ( {U}_{d , s , h} )}{{n}_{d , s , h} + 1} ]}^{\frac{1}{2}} .$$

wherein r_{d,s,h} is the regimen of dose level d, schedule s, and disease h,Pr(r_{d,s,h}=r_{d,s,h}=r_{d*,s*,h}) is the probability the regimen is the highest utility regimen, Var(U_{d,s,h}) is the variance of the regimen's disease-specific utility score, and n_{d,s,h} is the number of subjects already allocated to the regimen within disease cohort h.

71. The method of any of embodiments 1-70, wherein the clinical trial ends if:
none of the regimens have a first status of open and a second status of eligible; or
a total number of enrolled subjects is equal to or greater than a maximum subject enrollment limit;

72. The method of any of embodiments 1-71, wherein the clinical trial ends for one of the two or more diseases if:
none of the regimens for the disease have a first status of open and a second status of eligible;
a total number of enrolled subjects with the disease is equal to or greater than a maximum disease-subject enrollment limit;
response and toxicity information within the disease cohort has been determined for a first pre-determined minimum number of subjects within the regimen with the highest utility and the regimen is unlikely to be effective within the cohort for the purpose of termination; or
response and toxicity information within the disease cohort has been determined for a second pre-determined minimum number of subjects and the regimen with the highest utility is highly likely to be safe and effective within the cohort for the purpose of termination.

73. The method of embodiment 72 wherein a regimen is unlikely to be effective within the cohort for the purpose of termination if the probability that the efficacy rate is greater than a termination efficacy target is less than a termination efficacy probability parameter.

74. The method of embodiment 72 or 73, wherein a regimen is unlikely to be safe across the cohort for the purpose of termination if the probability that the toxicity rate is less than a termination safety limit is less than a termination toxicity probability parameter.

75. The method of any of embodiments 72-74, wherein a regimen is likely to be safe and effective within the cohort for the purpose of termination for early success if:
the probability that the toxicity rate is less than a success safety limit is greater than a success toxicity probability parameter; and
the probability that the efficacy rate is greater than a success efficacy target is greater than a success efficacy probability parameter.

76. The method of any of embodiments 1-75, wherein the therapeutic agent is one in which the response information can be determined within the same period of time in which the toxicity information is determined.

77. The method of embodiment 76, wherein the period of time is a length of time that is between about 1 day and about 7 days, between about 7 days and about 28 days, between about 28 days and about 42 days, between 42 days and about 84 days, or for a period of time greater than 84 days from the time the first dose of the therapeutic agent is administered in the regimen.

78. The method of embodiment 76, wherein the period of time is about 28 days starting from when the first dose of the therapeutic agent is administered in the regimen.

79. The method of embodiment 76, wherein the period of time begins when the first dose of the therapeutic agent is administered in the regimen, and extends between about 1 day and about 7 days, between about 7 days and about 28 days, between about 28 days and about 42 days, between 42 days and about 84 days, or for a period of time greater than 84 days from the time the last dose of the therapeutic agent is administered in the regimen.

80. The method of any of embodiment 76, wherein the period of time begins when the first dose of the therapeutic agent is administered in the regimen and extends for about 28 days after the last dose of the therapeutic agent is administered in the regimen.

81. The method of any of embodiments 76-80, wherein the overall utility score is calculated from response and toxicity information collected from one or more subjects previously treated with the therapeutic agent according to the treatment regimen after the period of time is completed.

82. The methods of any of embodiments 76-81, wherein the overall utility score is calculated from response information collected from one or more subjects within the same disease cohort and toxicity information collected from one or more subjects from one or more disease cohorts, wherein the one or more subjects were previously treated with the therapeutic agent according to the treatment regimen after the period of time is completed.

83. The method of any of embodiments 1-82, wherein the therapeutic agent comprises an adoptive cell therapy, a small molecule, a gene therapy, or a transplant.

84. The method of embodiment 83, wherein the small molecule is not a cytotoxic agent.

85. The method of any of embodiments 1-84, wherein the therapeutic agent comprises cells expressing a chimeric antigen receptor (CAR).

86. The method of any of embodiments 15-85, wherein the therapeutic agent comprises cells expressing a chimeric antigen receptor (CAR) that specifically binds to an antigen that is expressed by a cell or tissue of at least one of the diseases or disease subtypes, or that specifically binds to an antigen that is associated with at least one of the diseases or disease subtypes.

87. The method of any of embodiments 15-86, wherein at least one of the diseases or disease subtypes is a tumor or a cancer.

88. The method of any of embodiments 83-87, wherein the number of cells administered in the first dose is between about 0.5×10⁶ cells/kg body weight of the subject and 6×10⁶ cells/kg, between about 0.75×10⁶ cells/kg and 2.5×10⁶ cells/kg, between about 1×10⁶ cells/kg and 2×10⁶ cells/kg, between about 2×10⁶ cells per kilogram (cells/kg) body weight and about 6×10⁶ cells/kg, between about 2.5×10⁶ cells/kg and about 5.0×10⁶ cells/kg, or between about 3.0×10⁶ cells/kg and about 4.0×10⁶ cells/kg, each inclusive.

89. The method of any of embodiments 83-87, wherein the number of cells administered in the first dose is between about 1×10⁶ to 1×10⁸ CAR expressing cells, between about 2×10⁶ and about 5×10⁶ CAR expressing cells, between about 1×10⁷ and about 5×10⁷ CAR expressing cells, or between about 5×10⁷ and 1×10⁸ total CAR expressing cells.

90. The method of any of embodiments 83-89, wherein the dose of cells are administered in a single pharmaceutical composition comprising the cells of the dose.

91. The method of any of embodiments 83-90, wherein:
the dose is a split dose, wherein the cells of the dose are administered in a plurality of compositions, collectively comprising the cells of the dose, over a period of no more than three days.

92. The method of any of embodiments 86-91, wherein at least one of the diseases or disease subtypes is a leukemia or lymphoma.

93. The method of any of embodiments 86-92, wherein at least one of the diseases or disease subtypes is acute lymphoblastic leukemia.

94. The method of any of embodiments 1-93, wherein at least one of the diseases or disease subtypes is a non-Hodgkin lymphoma (NHL) or a subtype thereof.

95. The method of embodiment 94, wherein at least one of the disease subtypes is diffuse large B cell lymphoma (DLBCL).

96. The method of embodiment 94 or embodiment 95, wherein at least one of the disease subtypes is mantle cell lymphoma (MCL).

97. The method of any of embodiments 10-96 that is repeated for one or more additional subject enrolled in the clinical trial.

98. The method of any of embodiments 10-96, wherein the clinical trial is a phase I clinical trial.

99. A method of calculating an overall disease-specific utility score for each disease cohort in a treatment regimen, comprising:

a) determining a toxicity rate based on the toxicity information of subjects previously treated with a therapeutic agent according to a treatment regimen;

b) determining an efficacy rate based on the response information of subjects previously treated with the therapeutic agent according to the treatment regimen;

c) generating a safety utility score as a function of the toxicity rate for the treatment regimen;

d) generating an efficacy utility score as a function of the efficacy rate for the treatment regimen; and

e) multiplying the safety utility score by the efficacy utility score, thereby calculating the overall utility score for the treatment regimen.

100. The method of 99, wherein the treatment regimen is a treatment regimen for subjects in a population of subjects.

101. The method of embodiment 100, wherein the population of subjects are enrolled in a clinical trial.

102. The method of embodiment 101, wherein the clinical trial comprises an open enrollment design.

103. The method of embodiments 101 or 102, wherein the method further comprises reporting the overall disease-specific utility score for each disease cohort for each treatment regimen among the population of subjects.

104. The method of any of embodiments 99-104, wherein in step c):
if the toxicity rate is determined to be less than or equal to a first safety target, then the function defines the safety utility score as 1;
if the toxicity rate is determined to be greater or equal to a first safety limit, then the function defines the safety utility score as 0; and
if the toxicity rate is projected to be between the first safety target and the first safety limit, then the safety utility score decreases linearly as the toxicity rate increases.

105. The method of any of embodiments 99-104, wherein in step d):
if the efficacy rate is projected to be less than or equal to an efficacy target, then the function defines the efficacy utility score as 0;
if the efficacy rate is projected to be greater than the efficacy target, then the efficacy utility score increases linearly as the efficacy rate increases.

106. The method of any of embodiments 99-105, wherein the toxicity rate is a dose-limiting toxicity (DLT) rate.

107. The method of any of embodiments 99-106, wherein the efficacy rate is a complete response (CR) rate.

108. The method of any of embodiments 99-107, wherein the toxicity rate is determined by a regimen-toxicity model that a single toxicity rate for each regimen across all disease cohorts among the population of subjects.

109. The method of embodiment 108, wherein the toxicity rate is determined by a formula, whereby the formula is a logit function, wherein a first regimen is set as the referent, and wherein the logit function comprises the sum of the additive effects of the first regimen and of each additional regimen as compared to the first regimen.

110. The method of embodiment 109, wherein the additive effect of a regimen is constrained to be negative if the regimen comprises administering less of a total amount of the therapeutic agent than the first regimen, and wherein the additive effect of a regimen is constrained to be positive if the regimen comprises administering more of a total amount of the therapeutic agent than the first regimen.

111. The method of any of embodiments 108-110, wherein the regimen-toxicity model comprises the formula:

$$log ( \frac{π}{1 - π} ) = {β}_{0} + {β}_{1} {X}_{1} … + {β}_{n} {X}_{n}$$

wherein π is the DLT rate, wherein β₀ is a first regimen that is set as the referent, wherein β₁ X₁ . . . βₙXₙ are the additive effects of the remaining regimens in relation to the first regimen, and wherein is n is equal to the number of regimens minus one.

112. The method of any of embodiments 108-111, where the regimen-toxicity model comprises the formula

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {B}_{3} {1}_{[ s = 2 ⋂ d = - 1 ]}$$

113. The method of any of embodiments 99-112, wherein the efficacy rate is determined by a regimen-response model that estimates a single efficacy rate within each disease cohort for each regimen in the clinical trial.

114. The method of embodiment 113, wherein the regimen-response model borrows response information across disease cohorts.

115. The method of embodiment 113 or 114, wherein the efficacy rate is determined by a formula, whereby the formula is a logit function, wherein a first regimen is set as the referent, and wherein the logit function comprises the sum of the additive effects of the first regimen and of each additional regimen as compared to the first regimen.

116. The method of any of embodiments 113-115, wherein the additive effect of a regimen is constrained to be negative if the regimen comprises administering less of a total amount of the therapeutic agent than the first regimen, and wherein the additive effect of a regimen is constrained to be positive if the regimen comprises administering more of a total amount of the therapeutic agent than the first regimen.

117. The method of any of embodiments 113-116, wherein the regimen-efficacy model comprises the formula:

$$log ( \frac{θ}{1 - θ} ) = {α}_{0} + {α}_{1} {X}_{1} … + {α}_{n} {X}_{n}$$

wherein θ is the CR rate, wherein α₀ is a first regimen that is set as the referent, wherein α₁ X₁ . . . αₙXₙ are the additive effects of the remaining regimens in relation to the first regimen, and wherein is n is equal to the number of regimens minus one.

118. The method of embodiment 113-117, wherein the regimen response model comprises the formula

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

119. The method according to embodiment 113-118, wherein the response information is borrowed across disease cohorts according to the following distribution:

$${α}_{0 cohort 1} , {α}_{0 cohort 2} N ( θ , {σ}^{2} ) ; θ \sim N ( - 0.5 , {4}^{2} ) ; \frac{1}{{σ}^{2}} \sim Gamma ( 2 , 2 ) .$$

120. The method of any of embodiments 1-119, wherein the subject is a human subject.

121. The method of any of embodiments 1-120, wherein the method is a computer implemented method, and wherein one or more steps a)-c) occur at an electronic device comprising one or more processors and memory.

122. A computer system comprising a processor and memory, the memory comprising instructions operable to cause the processor to carry out any one or more of steps a)-c) of the methods of any of embodiments 1-98.

123. The method of any of embodiments 99-120, wherein the method is a computer implemented method, and wherein one or more steps occur at an electronic device comprising one or more processors and memory.

124. A computer system comprising a processor and memory, the memory comprising instructions operable to cause the processor to carry out any one or more of steps of the methods of any of embodiments 99-120 or 123.

125. A method for identifying an optimal treatment regimen in a clinical trial, comprising:
defining two or more different treatment regimens;
assigning prior values and distributions to each of the two or more treatment regimens, wherein the prior values comprise a mean toxicity rate and a mean efficacy rate, wherein the distributions comprise a credible intervals for the mean toxicity rate and credible intervals for the mean efficacy rate;
allocating a population of subjects to the two or more different treatment;
assessing and/or monitoring the subjects for toxicity and response events for a period of time that begins when the first dose of the therapeutic agent is administered to the subjects in the treatment regimen,
updating the prior values and calculating a utility score for each of the two or more treatment regimens based on toxicity and response events that are observed during the period of time in subjects who have completed one of the two or more treatment regimens;
identifying an optimal treatment regimen based on the utility scores calculated for each of the two or more treatment regimens; wherein the optimal treatment regimen has acceptable efficacy and toxicity rates.

### VII. EXAMPLES

The following examples are included for illustrative purposes only and are not intended to limit the scope of the invention.

### Example 1 Design of a Clinical Trial

A multicenter phase 1 clinical trial was designed to assess and optimize both safety and efficacy of a therapeutic agent, such as a cellular immunotherapy, on a mixed population by adaptively assigning subjects to different dosing regimens. The trial was designed to include open enrollment, meaning that patients are enrolled as they become available for the trial. The open enrollment has advantages over cohort enrollment because no or few pauses are required while waiting for the completion of dose-limiting toxicity (DLT) evaluation period for all patients in the cohort. The trial was designed so that subjects are assigned to one of four possible investigational dosing regimens used to treat two different disease subtypes. The assignments are adaptively based on previously determined outcomes of subjects who have already completed certain phases of the trial. This example describes design of the method in connection with executing a phase I trial of CD19-targeted chimeric antigen receptor T-cells (CAR-T) therapy for non-Hodgkin lymphoma (NHL) testing using multiple dosing regimens in multiple NHL subtypes. The method can be adapted to design phase I clinical trials for other therapies, and in particular therapies in which toxicity and efficacy response can be measured in the same timeframe.

In performing the method, a Bayesian adaptive design was developed to simultaneously consider safety and efficacy with sharing of information between NHL disease subtypes to determine an optimal dose and schedule (regimen). The design was based on designating four treatment regimens (two dose levels, each given in either a one or two dose administration schedule) in two NHL disease subtypes, diffuse large B cell lymphoma (DLBCL) and mantle cell lymphoma (MCL) as follows:
Regimen, r1,1: Dose Level 1, single dose Schedule
Regimen, r1,2: Dose Level 1, 2-dose Schedule
Regimen, r−1,1: Dose Level −1, single dose schedule
Regimen, r−1,2: Dose Level 1, 2-dose schedule.

The design designated the starting regimen to be Dose Level 1, single-dose schedule. Dose Level −1, single-dose schedule was set as a de-escalation regimen. Among the three other designated treatment regimens, Dose Level 1, 2-dose schedule and Dose Level −1, 2-dose schedule were set as escalation regimens.

The model was designed to consider both toxicity and efficacy responses. This is because, for certain therapeutic agents, toxicity and efficacy response can be determined and optimized simultaneously. For example, a complete response (CR) to CAR-T therapy is an efficacy event that usually appears rapidly, and generally in the same timeframe as information about toxicity events, such as dose-limiting toxicity (DLT).

In the design of the model, the model assumed that DLT is a toxicity event that is drug dependent rather than disease dependent. Therefore, DLT information was pooled across disease subtypes using a single regimen-toxicity model for the analysis.

The model also assumed that efficacy data is likely to be disease specific. Thus, CR information was modelled separately and so that information is shared between diseases, which allows for disease-specific estimates of efficacy.

The model was designed to calculate an overall disease-specific utility score for each regimen using the toxicity (e.g. DLT) and response (e.g. CR) information. The utility score balances toxicity with response information (e.g. antitumor activity) so fewer patients will be allocated to regimens that appear to be unsafe or ineffective.

In practicing the model in the context of the exemplary clinical trial assessing (CAR-T) therapy for NHL, toxicity and efficacy can be assessed for a period of time that begins with the first dose of each dosing regimen and ends 28 days after the last dose of the dosing regimen. Thus, the assessment period ends 28 days after the first dose in the case of a single-dose regimen, or 28 days after the second dose in the case of a two-dose regimen.

A. Selection of Prior Distribution

The models for determining regimen toxicity and efficacy were designed with prior values/distributions chosen to be relatively non-informative while still reflecting expected prior beliefs of the probability of a DLT and CR for each dose regimen. The chosen mean DLT and CR rates and 95% credible intervals for each of the dose regimens are presented in Table 3.

TABLE 3
Mean Prior DLT and CR rates and 95% credible intervals

| | | | | | Efficacy (Within each | | |
|---|---|---|---|---|---|---|---|
| Regimen | | Safety | | | Disease Cohort) | | |
| Dose | | | Lower | Upper | | Lower | Upper |
| Level | Schedule | Mean | 95% CI | 95% CI | Mean | 95% CI | 95% CI |
| −1 | Single | 0.05 | 0.01 | 0.30 | 0.30 | 0.01 | 0.87 |
| −1 | 2-dose | 0.06 | 0.01 | 0.40 | 0.45 | 0.02 | 0.96 |
| 1 | Single | 0.17 | 0.01 | 0.68 | 0.41 | 0.04 | 0.92 |
| 1 | 2-dose | 0.21 | 0.01 | 0.77 | 0.57 | 0.07 | 0.97 |

The models were designed so the prior for the regimen-response model within each disease cohort is the same. These priors were designed to put the highest probability (the prior mode) on the belief that all regimens are likely to be safe (DLT rate<0.33) and most are likely effective (CR rate>0.40). The model assumed that a regimen with a posterior mode outside this range would not be suitable for the trial.

However, the priors were designed to be relatively non-informative and stretch into the unsafe or non-efficacious range. The 95% CI for the DLT rate for the starting dose regimen (Dose Level 1, single Schedule) is between 0.01 and 0.68. The 95% CI for the CR rate for the start dose regimen is between 0.04 and 0.92. Thus, the priors were designed to allow for a wide range of possibilities, and include sufficiently high probability that the true DLT rate may lie in the unsafe (DLT rate>0.33) range.

The priors selected for the model were designed to produce ethical and efficient changes in doses and regimens based upon accruing safety and efficacy data—much like a 3+3 design produces desirable operating characteristics. The priors were designed to influence the dose selection, particularly at the beginning of the trial with few patients enrolled, and as such the values of the parameters contributing to the priors were chosen based upon their ability to produce desirable design features. The prior values were chosen via extensive clinical trial simulation and inspection within individual trials to investigate and determine the types of outcomes that lead to dose/regimen escalation and de-escalation. The selected priors provided for desirable regimen escalation or de-escalation behavior at the beginning of the trial with little observed data.

B. Determining Regimen Toxicity

The model was designed for a trial in which a dosing regimen is deescalated when toxicity is determined to be too high. A regimen-toxicity model was employed to pool safety data across disease cohorts and to estimate a single probability of a toxicity event (e.g. DLT, also called a DLT rate) for each regimen across disease cohorts. For each dose level (d=−1,1) and schedule (s=1,2) the DLT rate (η_{d,s}) is modeled on the log odds scale as

$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {B}_{3} {1}_{[ s = 2 ⋂ d = - 1 ]}$$

β₁ is an additive effect due to dose level. The model was parameterized so that dose level 1 is the referent and so β₁ is constrained to be negative, reflecting a decrease in DLT rates going from dose level 1 to dose level −1. β₂ is an additive effect due to adding a second dose of dose level 1. β₃ is an additive effect due to adding a second dose of dose level −1. The model was parameterized so that a single-dose schedule is the referent and so β₂ and β₃ are constrained to be positive, reflecting an increase in the DLT rate of increasing from a single-dose schedule to a 2-dose schedule. Thus, β₀, the intercept term captures the log-odds of DLT rate for dose level 1, single-dose schedule of administration.

The following prior distributions were placed on each of the terms:

β₀˜N(−2.2,1.5²);β₁˜N_{(-∞,0]}(−2,1²);β₂˜N_{[0,∞)}(−10,2²);β₃N_{[0,∞)}(−10,2²).

The model was designed so that the joint posterior distributions of all model parameters within the regimen-toxicity model are fit and updated after DLT information is obtained on each subject using Markov chain Monte Carlo. In particular, samples from the posterior distributions of the regimen-specific DLT rates, π_{d,s}, are calculated. Posterior means and 95% credible intervals are provided as estimates of each.

The following posterior summaries were also provided, which are calculated by tabulating the proportion of samples from the posterior distribution of n_{d,s} that are less than 33% or 20%:

Probability DLT Rate is less than 33%: Pr(π_{d,s}<0.33);

Probability DLT Rate is less than 20%: Pr(π_{d,s}<0.20).

C. Determining Regimen Response

A regimen-response model was employed to estimate the different probabilities of an efficacy event (e.g. CR, also called a CR rate) across dose regimens within the different disease cohorts. In particular, for each dose level (d=−1,1), schedule (s=1,2), and disease cohort (h=DLBCL,MCL), the CR rate (θ_{d,s,h}) is modeled on the log odds scale as:

$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

The regimen-response model was parameterized similarly to the regimen-toxicity model. The dose level referent is dose level 1 and the schedule referent is a single-dose schedule. α1, the additive effect due to dose level is constrained to be negative and α2 and α3, the additive effects due to schedule are constrained to be positive.

The following prior distributions were placed on each of the regimen-response terms:

α₁˜N_{(-∞,0]}(2,1.5²);α₂˜_{[0,∞)}(−1,1.5²);α₃˜N_{[0,∞)}(−1,1.5²).

The intercept term and estimate for the referent regimen is unique within the different disease cohorts. However, efficacy information is borrowed rather than pooled across disease cohorts. The following hierarchical prior distribution was placed on the parameters:

$${α}_{0 , DLBCL} , {α}_{0 , MCL} \sim N ( θ , {σ}^{2} ) ; θ \sim N ( - 0.5 , {4}^{2} ) ; \frac{1}{{σ}^{2}} \sim Gamma ( 2 , 2 ) .$$

As with toxicity, the model was designed so that the joint posterior distributions of all model parameters within the regimen-response models are fit and updated after CR information is obtained on each subject using Markov chain Monte Carlo. Samples from the posterior distributions of the regimen and disease cohort-specific CR rates, θ_{d,s,h} are calculated. Posterior means and 95% credible intervals are provided as estimates of each. The following posterior summaries are also provided for each regimen and disease cohort, which are calculated by tabulating the proportion of samples from the posterior distribution of θ_{d,s,h} that are greater than 25% or 40%.

Probability CR Rates is greater than 25%: Pr(θ_{d,s,h}0.25);

Probability CR Rate is greater than 40%: Pr(θ_{d,s,h}>0.40).

D. Determining a Utility Score

A utility function for safety and efficacy was constructed based on target safety and efficacy parameters to determine a safety utility score and an efficacy utility score, respectively, for each treatment regimen. FIG. 1 depicts an exemplary function for determining the utility safety score (FIG. 1A) and the utility efficacy score (FIG. 1B).

In this exemplary model, the utility safety score was defined as 1 if the DLT rate is less than or equal to 20%. Conversely, the utility safety score was defined as 0 if the DLT rate is greater than 33%. Between a 20% and 33% DLT rate, utility decreases linearly as the DLT rate increases. Specifically, safety utility decreases 0.77 units for every 10% increase in the DLT rate.

In this exemplary model, the utility efficacy score was defined as 0 if the CR rate is less than 25%. Beyond a 25% CR rate, utility was defined to increase linearly from 0 to 1 as the CR rate increases linearly from 25% to 100%. Specifically, safety utility increases at the rate of a 0.13-unit increase in utility for every 10% increase in the CR rate.

Based on the modeling described above, samples from the posterior distribution will exist for each regimen-specific DLT and CR rate. For each of those samples from the posterior distribution, each regimen has a corresponding utility value for safety and efficacy. To obtain an overall disease utility score for each subtype in each treatment regimen, these scores are multiplied in order to create a distribution of the regimen's overall utility scores. The mean of overall utility scores calculated from each posterior distribution for each regimen is then reported.

E. Adaptively Assigning a Subject to a Particular Regimen

The clinical trial was designed to adaptively allocate entering patients into specific treatment regimens based on known safety and efficacy data for each regimen and disease according to the calculated overall disease utility score for each disease subtype and each treatment regimen as described above. In the trial, each of the four regimens is either eligible or ineligible for accepting new patients. The criteria for determining if a regimen is eligible for accepting new patients include:
whether the regimen is closed or open;
whether the regimen is suspended or not suspended;
the number of patients enrolled in the regimen; in the exemplary model the total study is limited to 60 patients, and each disease cohort is limited to 35 patients; and
the number of patients for whom complete toxicity data are known.

5. Criteria for Opening a Regimen

An entering patient can only be allocated to an open regimen. In the design, the clinical trial is conducted by initially allocating all patients to the dose level 1, single dose schedule treatment regimen. Other regimens can be opened after 3 patients have completed the DLT period across disease cohorts on the initial regimen. A regimen can be opened based on either safety or efficacy criteria, which are considered individually for the purpose of opening a regimen. The rules for opening a regimen based on safety are shown in Table 4 and the rules for opening a regimen based on efficacy are shown in Table 5. In this exemplary design, the rules for opening a regimen are different from and independent of the rules for suspending a regimen.

TABLE 4
Decision Rules to Open Regimens based on Safety

| | Dose Level 1 | Dose Level −1 |
|---|---|---|
| Single-dose | Initial regimen | A dose level −1, single-dose regimen is opened |
| schedule | | for all disease cohorts when the initial regimen |
| | | has at least 3 completers and is unlikely to be safe |
| | | across disease cohorts. This is a de-escalation. |
| | | *If Dose level −1 under the 2-dose schedule is |
| | | already open and not unlikely to be safe, there is no |
| | | de-escalation. |
| 2-dose | A dose level 1, two-dose regimen is opened | OPTION 1: |
| Schedule | when the initial regimen has 12 enrolled | A dose level −1, 2 dose schedule regimen is opened |
| | subjects within a disease cohort and dose | when the dose level 1, 2-dose schedule is unlikely to |
| | level 1, single-dose schedule is likely to be | be safe and the initial regimen is likely to be safe |
| | safe across cohorts. This is an escalation | across disease cohorts. This is a de-escalation from |
| | from the dose level 1, single-dose regimen. | the dose level 1, 2 dose regimen. |
| | | OPTION 2: |
| | | A dose level −1, 2 dose schedule regimen is opened |
| | | when the dose level −1, single-dose schedule has at |
| | | least 12 enrolled subjects within a disease cohort |
| | | and the dose level −1, 2-dose schedule is likely to be |
| | | safe across cohorts. This is an escalation from the |
| | | dose level −1, 2-dose regimen |

TABLE 5
Decision Rules to Open Regimens based on Efficacy

| | Dose Level 1 | Dose Level −1 |
|---|---|---|
| Single- | Initial regimen | — |
| dose | | |
| schedule | | |
| 2-dose | A dose level 1, 2 dose schedule regimen | A dose level −1, 2 dose schedule regimen is |
| schedule | is opened when the initial regimen (dose | opened when there are at least 3 completers at |
| | level 1, single-dose) has at least 3 | dose level −1, single-dose schedule and that |
| | completers and is unlikely to be effective | regimen is unlikely to be effective within a |
| | within a disease cohort. This is an | disease cohort. This is an escalation from dose |
| | escalation from the initial regimen. | level −1, single dose regimen. |

For the purposes of opening a regimen in this exemplary clinical trial design, a regimen is considered unlikely to be safe if there is less than a 30% probability the DLT rate is less than 20%:

Pr(π_{d,s}<20%)<30%,

According to this exemplary model, the above quantity is used to make decisions on when the trial should de-escalate and a lower dosing regimen should be opened. A DLT rate of 20% was chosen for this exemplary model to reflect the desire be conservative by increasing the likelihood of selecting a regimen with a true DLT rate less than 33%.

A regimen is considered unlikely to be effective in this exemplary model if there is less than a 30% probability that the CR rate is greater than 40%:

Pr(θ_{d,s,h}>40%)<30%.

6. Criteria for Suspending a Regimen Based on Safety and Efficacy Criteria

The design is modeled so that, in some cases, once a regimen is open, it can be temporarily suspended from accepting new patients if the regimen is unlikely to be safe across disease cohorts or efficacious within a particular disease cohort. In this exemplary clinical trial design, the rules were designed so that a regimen will be temporarily suspended for safety across cohorts if it has a DLT rate greater than 33% and if there is less than a 50% probability that the DLT rate is less than 33% (Pr(π_{d,s}<33%)<50%).

There are two ways a regimen can be suspended based on efficacy criteria. First, enrollment to a regimen may be temporarily suspended for futility within a disease cohort. A regimen will be temporarily suspended if a regimen has 6 completers and has an observed utility score<5% within a disease cohort.

Second, enrollment to a regimen may be temporarily suspended if it is deemed an early success within a disease cohort, but if another regimen has a higher utility score. Specifically, enrollment to a regimen will be temporarily suspended for early success if it is not the highest utility regimen, has 12 completers within a disease cohort, and is highly likely to be both safe (Pr(π_{d,s}<33%)>70%) and effective (Pr(θ_{d,s,h}>25%)>90%).

In practice of the design, suspensions of enrollment to regimens for safety, futility, or early success can be temporary. A suspension can be lifted, making the regimen eligible for enrollment again, if additional patient outcomes for a particular regimen or cohort include patient outcomes with complete responses or lacking toxicity.

7. Criteria for Limiting the Number of Patients Assigned to a Regimen Based on the Number of Patients with Known and Unknown Toxicity Profiles

The accrual rate into the trial is governed by limiting the number of patients enrolled in each regimen with unknown DLT information. Initially, no more than three patients with unknown DLT information are allowed in a particular regimen. Once there are six patients with complete DLT information, the number of patients with unknown DLT information allowed in a particular regimen increases to four. As described above, DLT information is pooled across disease cohorts for a particular regimen. Thus, whether a patient can enter a particular regimen is dependent on both the number of patients with complete DLT information and the number of patients with unknown DLT information across disease cohorts.

8. Adaptive Randomization of Patients to Open and Eligible Regimens

In the model for allocating subjects to a clinical trial, all patients are assigned to a single regimen if only a single regimen is open and eligible for enrollment. If more than one regimen is open, patients are adaptively randomized to all regimens that are open and eligible for enrollment. Allocation to open and eligible regimens is based on the disease cohort-specific utility for each regimen as calculated above, the relative uncertainty of the estimate for the utility of each regimen, and the number of subjects already allocated to the regimen within the disease cohort. In particular, the randomization probability of enrollment to each regimen within each disease cohort, V_{d,s,h}, is set as:

$${V}_{d , s , h} ∝ {[ \frac{Pr ( {r}_{d , s , h} = {r}_{{d}^{*} , {s}^{*} , h} ) Var ( {U}_{d , s , h} )}{{n}_{d , s , h} + 1} ]}^{\frac{1}{2}} .$$

Where r_{d,s,h} represents the regimen of dose level d, schedule s, and disease h, and Pr(r_{d,s,h}==r_{d*,s*,h}) is the probability the regimen is the highest utility regimen, Var(U_{d,s,h}) is the variance of the regimen's estimated disease-specific utility score, and n_{d,s,h} are the number of patients already allocated to the regimen within disease cohort h.

The model was designed to balance allocating patients to regimens to increase the certainty of utility estimates while also preferentially allocating subjects to regimens with the highest utility. The model was designed so that randomization probabilities will be more dispersed across the open regimens if there is a large amount of uncertainty in the estimates of the utility for each dose. However, regimens with higher utility receive a greater randomization probability because randomization probabilities will peak at the highest utility dose as the accumulating data increase the certainty of utility estimates for each open regimen. This balance was designed to ensure that randomization probabilities will not focus on a single regimen prematurely if there is still a large amount of uncertainty as to which regimen has the highest utility.

The model was also designed in this manner because it is statistically efficient. For example, in a 2-arm trial, maximum efficiency comes from 1:1 randomization. If two regimens are performing similarly in terms of utility, the regimen with the smaller sample size and/or higher variance is favored for enrollment. Increasing the randomization probability to the regimen with the smaller sample size steers the trial back towards equal randomization between the best regimens and maximum statistical efficiency.

F. Criteria for Stopping the Clinical Trial Early

In the model, the trial will continue as long as at least one disease cohort is enrolling or until the maximum total sample size of 60 patients across the disease cohorts is reached. The trial is continuously monitored and may be stopped early for either futility or success.

Each regimen will be individually assessed by disease cohort for a need to stop the trial early due to futility. If a first disease cohort meets the enrollment stopping criteria, but a second disease cohort does not, the trial will only continue enrolling patients into the second cohort until the enrollment stopping criteria is met. The trial will stop early for futility within a disease cohort if either: i) no regimens are eligible for enrollment within that cohort; or ii) the highest open utility regimen within that cohort has at least 6 completers and is unlikely to be effective within that cohort. In this exemplary model, a regimen is defined as unlikely to be effective when the probability that a regimen will achieve at least a 25% complete response rate, i.e., efficacy rate, for a particular disease cohort is less than 50% (Pr(θ_{d*,s*,h}>25%)<50%). A disease cohort may also be stopped if the maximum sample size of 35 patients enrolled within the cohort is reached.

Each regimen will also be individually assessed by disease cohort for a need to stop the trial early due to success. The trial will stop early for success within a disease cohort if there are at least 12 completers on the highest open utility regimen and is highly likely to be both safe and effective within that cohort. A regimen is defined as highly likely to be both safe and effective within a cohort when the probability that a regimen will achieve a DLT rate of less than 33% is greater than 70% and the probability that a regimen will achieve a complete response rate, i.e. efficacy rate, of at least 25% for a particular disease cohort is greater than 90% (e.g. Pr(π_{d*,s*}<33%)>70% and Pr(θ_{d*,s*,h}>25%)>90%). These stopping rules are exemplary, and were determined based on experiences with existing CAR-T therapies.

G. Criteria for Determining if the Clinical Trial is a Success

The clinical trial will be deemed a success if the regimen with the highest utility score for a particular disease cohort has a probability that the regimen will achieve a DLT rate of less than 33% that is greater than 50% and the probability that the regimen will achieve a complete response rate, i.e., efficacy rate, of at least 25% for a particular disease cohort is greater than 80%. Thus, the criteria for stopping a study for early success are higher than the criteria for determining if a completed trial was a success.

### Example 2 Example Clinical Trial Results

The behavior of the clinical trial model described above was assessed in a simulated clinical trial to assess both the safety and efficacy of an immunotherapy for treating two different non-Hodgkin lymphomas, diffuse large B-cell lymphoma (DLBCL) and mantle cell lymphoma (MCL). The simulated clinical trial enrolled subjects with one of the two lymphomas on a rolling basis. Simulated subjects were adaptively assigned to receive a therapeutic regimen of a particular dose amount and dose schedule based on the outcomes of earlier-enrolled patients. Specifically, the safety of the immunotherapy in the simulated trial was tested across all subjects receiving the immunotherapy, regardless of lymphoma type, while the efficacy of the immunotherapy was tested within patients with the same lymphoma type. For all patients enrolled in the simulated trial, relevant statistics were calculated and graphed to assist dose assignment for the next patient that enters the trial.

The simulated trial began with patients assigned to the initial regimen, Dose Level 1, single-dose schedule. A total of 9 (7 DLBCL and 2 MCL) patients entered the simulated trial. Initially, 5 DLBCL and 1 MCL patients received the dose level 1, single dose regimen. After these first six patients have completed the DLT and efficacy evaluation period, the remaining three patients (2 DLBCL and 1 MCL) were enrolled in this regimen.

At this point in the simulated trial, the possibility of opening other regimens was assessed. One patient experienced a DLT, so this initial regimen was considered safe and de-escalation for safety was not necessary. Two of seven DLBCL patients achieved a CR and zero of two MCL patients achieved a CR, so escalation for lack of efficacy was not necessary. The simulated trial continued and all patients continued to be assigned to Dose Level 1, single-dose schedule.

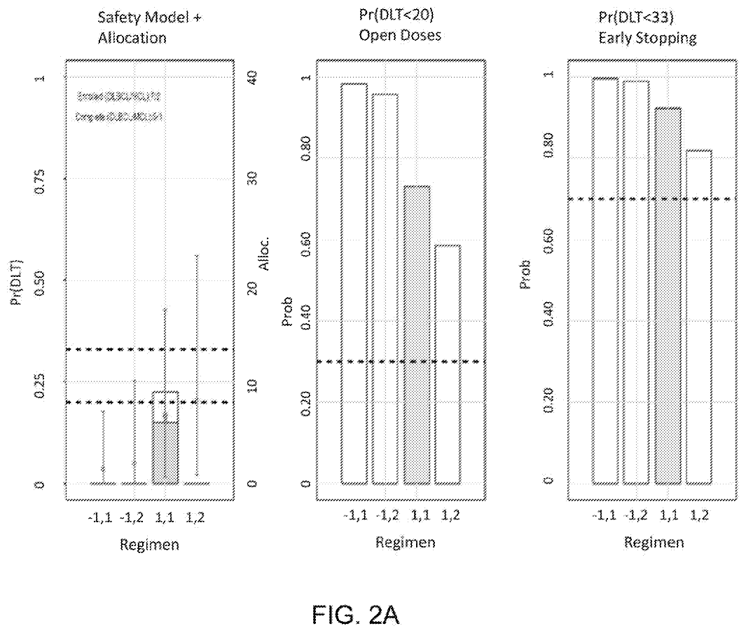

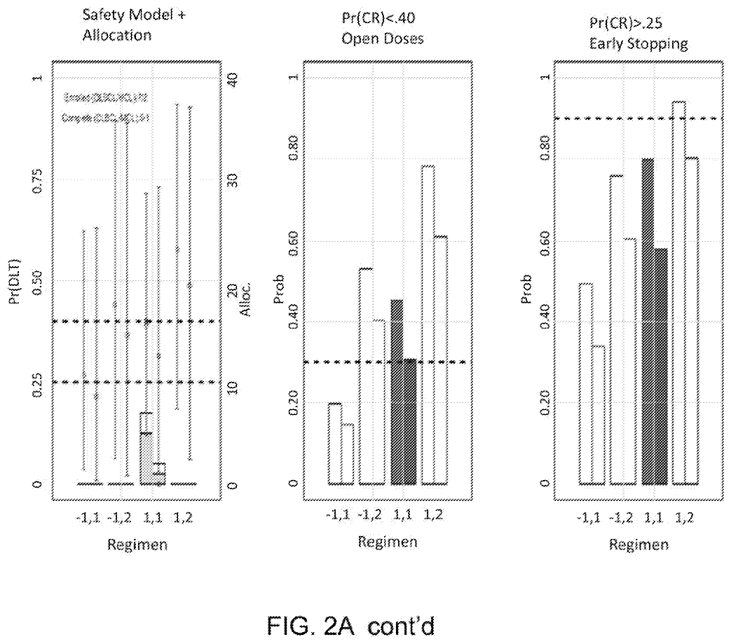

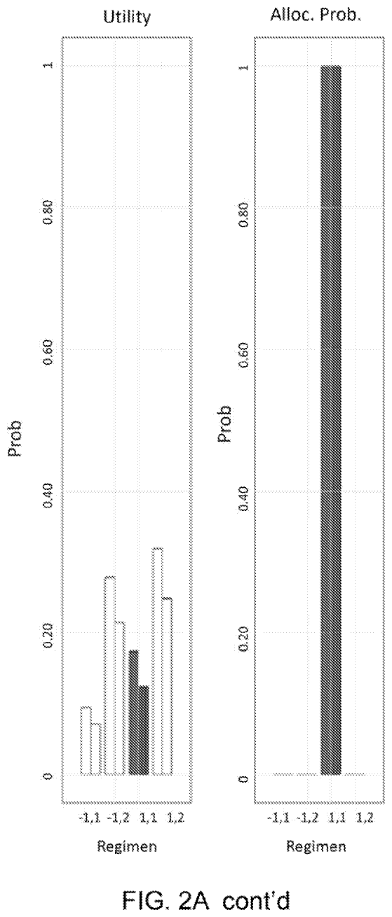

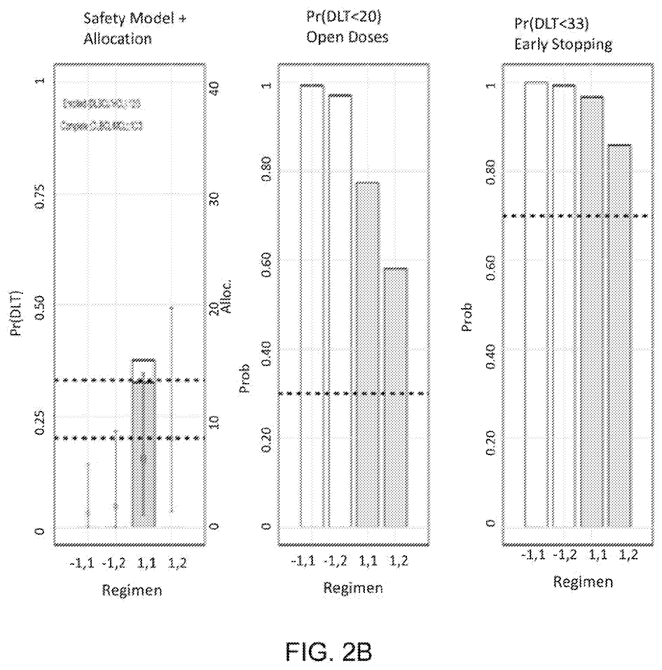

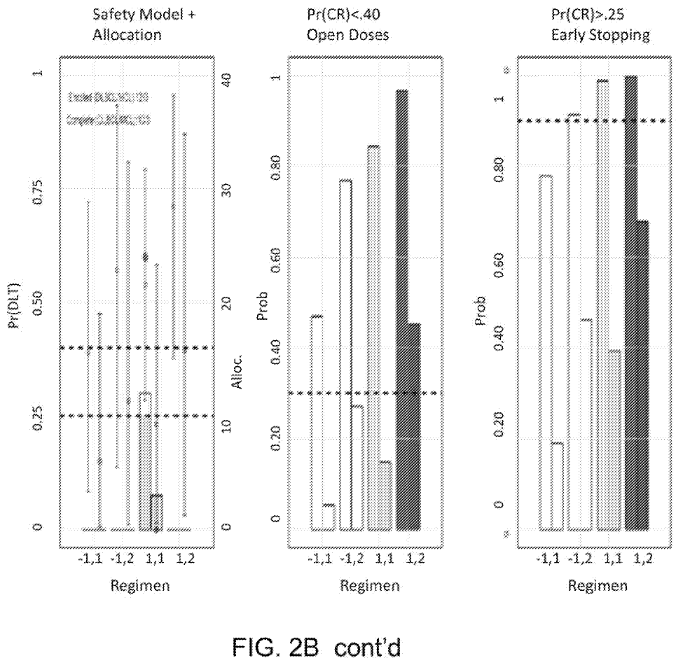

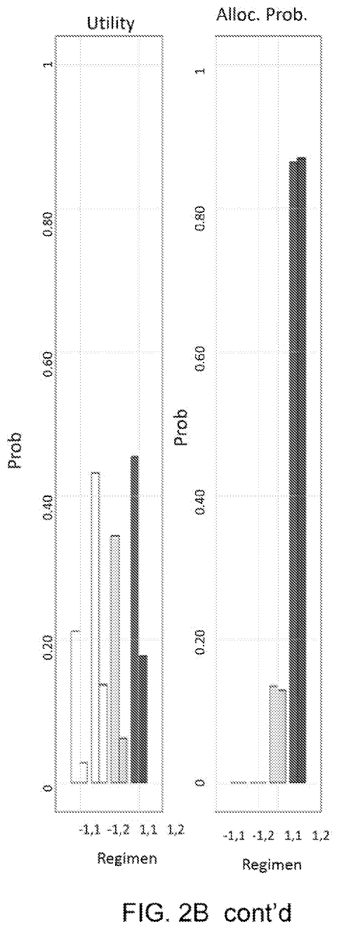

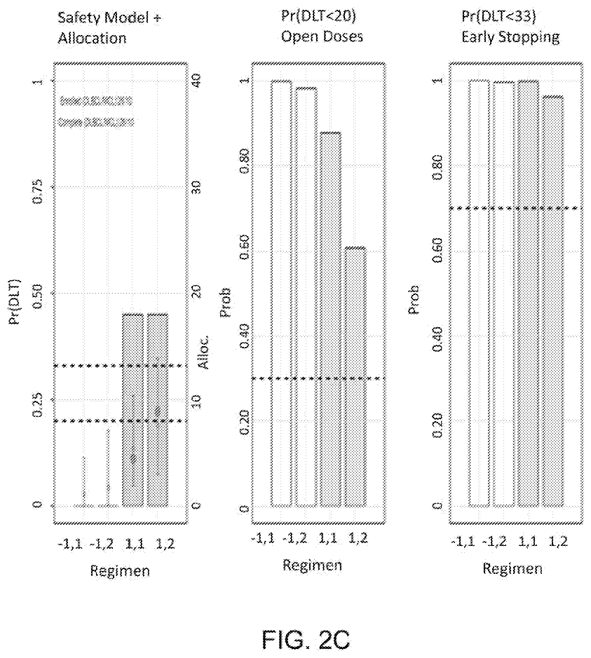

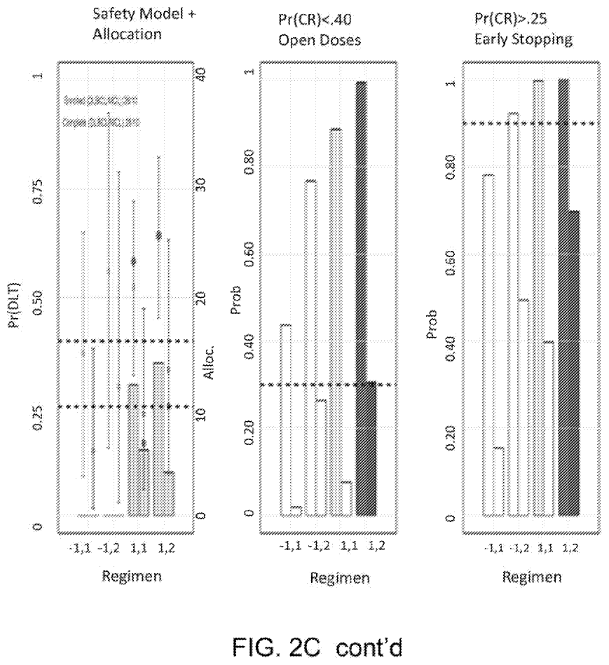

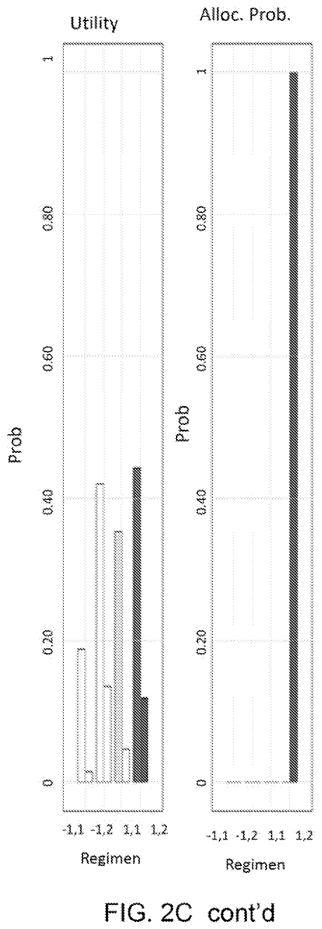

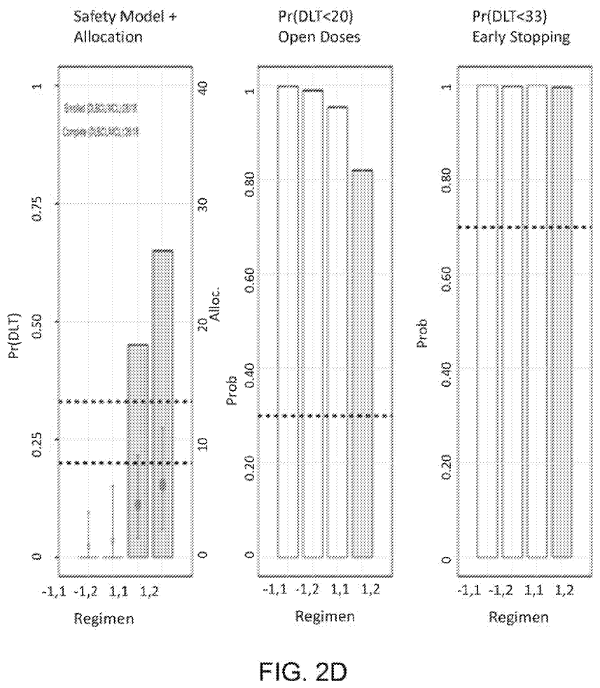

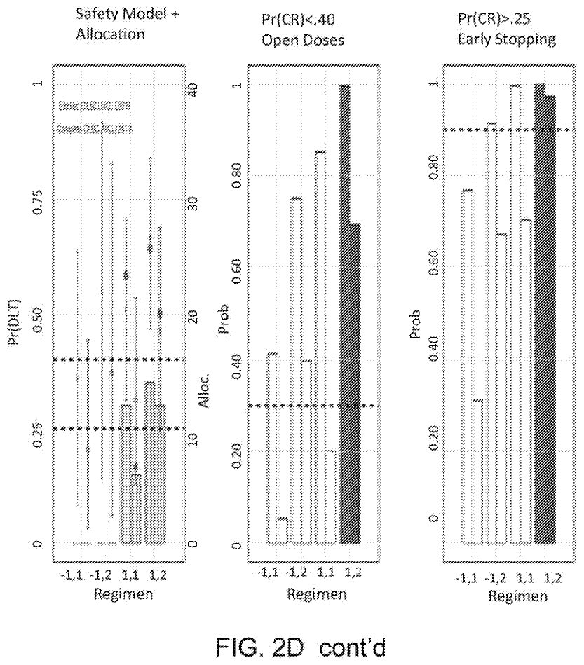

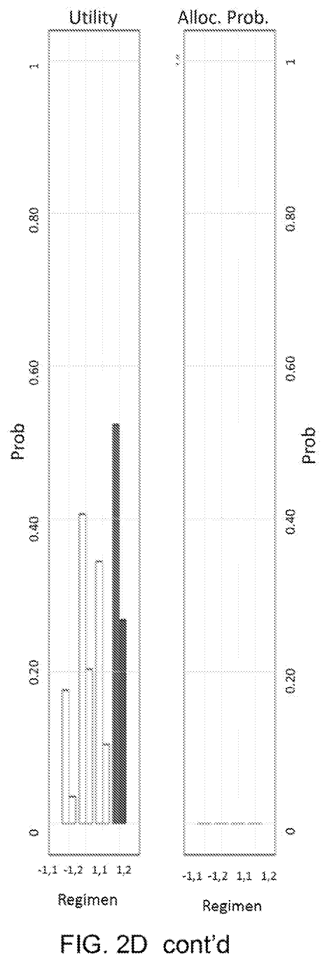

When 10 DLBCL and 3 MCL patients completed the DLT and efficacy evaluation period in the simulated trial, a total of 15 (12 DLBCL and 3 MCL) patients were enrolled to Dose level 1, single-dose schedule. Results are shown in FIG. 2B. In the simulated trial, two patients out of 13 experienced a DLT across disease cohorts and, at this stage, all open regimens were considered safe. Six out of 10 DLBCL patients and zero out of three MCL patients achieved a CR.

Since the initial regimen had 12 patients enrolled within DLBCL and Dose level 1, 2-dose schedule was determined as likely safe, the simulated trial escalated the dose and opened Dose level 1, 2-dose schedule for enrollment within DLBCL. Because the initial regimen had 3 completers within MCL and was unlikely to be effective, the simulated trial also escalated and opened Dose level 1, 2-dose schedule for enrollment within MCL.

Because both Dose level 1, single- and 2-dose schedule regimens were open and eligible for enrollment within both disease cohorts, the next patient in the simulated trial was randomized based on the overall utility score of the open regimens and the corresponding randomization probability within each cohort. Based on the rules for allocating a subject to a treatment regimen, it was found that a DLBCL or an MCL patient was about 8 times more likely to randomize to the 2-dose schedule compared to the single-dose schedule.

The simulated trial expanded to a total of 36 (26 DLBCL and 10 MCL) enrolled patients. At this point, 18 patients (12 DLBCL and 6 MCL) at Dose level 1, single-dose schedule and 18 patients (14 DLBCL and 4 MCL) at Dose level 1, 2-dose schedule completed the DLT and efficacy evaluation. Results are shown in FIG. 2C. In the simulated results, two patients out of 16 had a DLT at Dose level 1, single-dose schedule and four patients out of 16 had a DLT at Dose level 1, 2-dose schedule. Therefore, all regimens were likely to be safe. For subjects with DLBCL, dose level 1, 2-dose schedule was the open regimen with the highest overall utility score. In the simulated trial, eight out of 14 DLBCL patients achieved CR on this regimen, and this regimen was likely to be safe and effective (Pr(π_{1,2}<33%)>70% and Pr(θ_{1,2,DLBCL}>25%)>90%). Thus, the simulated trial stopped early for success within the DLBCL disease cohort and enrollment was continued for only MCL patients.

For subjects with MCL, dose level 1, single-dose schedule had 6 MCL completers and an overall utility score of <5%. Thus, this regimen was suspended for MCL due to futility, and all MCL patients were temporarily enrolled to the 2-dose schedule.

Additional MCL patients were enrolled in the simulated trial. When 12 MCL patients completed the DLT and efficacy evaluation at Dose level 1, 2-dose schedule, 6 patients achieved a CR and the regimen was determined as likely safe and effective (Pr(π_{1,2}<33%)>70% and Pr(θ_{1,2,MCL}>25%)>90%). The simulated trial was stopped early due to success within MCL. The results are shown in FIG. 2D.

The simulated trial was able to identify a safe and effective regimen for both DLBCL and MCL using a total sample size of 44 patients (28 DLBCL and 16 MCL). For DLBCL, the simulated trial identified Dose level 1, single- and 2-dose schedules as safe and effective; for MCL, the simulated trial identified Dose level 1, single-dose schedule as safe but not effective, and Dose level 1, 2-dose schedule as safe and effective.

### Example 3 Simulating Outcomes Using a Bayesian Adaptive Phase I Clinical Trial Design

To further characterize the design, varying assumptions for safety and efficacy within each disease cohort were used to create different scenarios and sub-scenarios in which simulated trials were run. Table 6 depicts the varying assumptions that were used. For the varying scenarios, the simulated trial assumed that safety was the same in DLBCL and MCL. The assumptions used for the probability of DLT and CR (the rate of DLT or the rate of CR) are shown in Table 5. Regimens considered both safe and effective are in bold and italics. All simulations assumed that patients are accrued at the rate of 4 per months with 75% of the patients with DLBCL and 25% with MCL.

TABLE 6
Dose-Toxicity and Dose-Response Simulation Scenarios

| | | | | Pr (CR) | | | | Utility | | | |
|---|---|---|---|---|---|---|---|---|---|---|---|
| | | | | | Sub-scenario | | | | Sub-scenario | | |
| | Dose | | | | .1 | .2 | .3 | | .1 | .2 | .3 |
| Scenario | Level | Schedule | Pr(DLT) | D | M = D | M < D | M > D | D | M = D | M < D | M > D |
| 1.1-1.3 | 1 | 1-dose | 0.10 | 0.5 | 0.5 | 0.29 | 0.73 | [symbol] | [symbol] | 0.05 | [symbol] |
| | 1 | 2-dose | 0.14 | 0.6 | 0.6 | 0.38 | 0.8 | [symbol] | [symbol] | [symbol] | [symbol] |
| | −1 | 1-dose | 0.05 | 0.18 | 0.18 | 0.08 | 0.38 | 0 | 0 | 0 | [symbol] |
| | −1 | 2-dose | 0.08 | 0.25 | 0.25 | 0.12 | 0.48 | 0 | 0 | 0 | [symbol] |
| 2.1-2.3 | 1 | 1-dose | 0.38 | 0.5 | 0.5 | 0.29 | 0.73 | 0 | 0 | 0 | 0 |
| | 1 | 2-dose | 0.48 | 0.6 | 0.6 | 0.38 | 0.8 | 0 | 0 | 0 | 0 |
| | −1 | 1-dose | 0.15 | 0.43 | 0.43 | 0.23 | 0.67 | [symbol] | [symbol] | 0 | [symbol] |
| | −1 | 2-dose | 0.21 | 0.52 | 0.52 | 0.31 | 0.75 | [symbol] | [symbol] | [symbol] | [symbol] |
| 3.1-3.3 | 1 | 1-dose | 0.1 | 0.23 | 0.23 | 0.11 | 0.45 | 0 | 0 | 0 | [symbol] |
| | 1 | 2-dose | 0.14 | 0.45 | 0.45 | 0.25 | 0.69 | [symbol] | [symbol] | 0 | [symbol] |
| | −1 | 1-dose | 0.05 | 0.17 | 0.17 | 0.08 | 0.35 | 0 | 0 | 0 | [symbol] |
| | −1 | 2-dose | 0.08 | 0.23 | 0.23 | 0.11 | 0.45 | 0 | 0 | 0 | [symbol] |
| 4.1-4.3 | 1 | 1-dose | 0.38 | 0.27 | 0.27 | 0.13 | 0.5 | 0 | 0 | 0 | 0 |
| | 1 | 2-dose | 0.48 | 0.35 | 0.35 | 0.18 | 0.6 | 0 | 0 | 0 | 0 |
| | −1 | 1-dose | 0.15 | 0.18 | 0.18 | 0.08 | 0.38 | 0 | 0 | 0 | [symbol] |
| | −1 | 2-dose | 0.21 | 0.25 | 0.25 | 0.12 | 0.48 | 0 | 0 | 0 | [symbol] |

D=DLBCL, M=MCL

The simulations were based on a clinical trial containing the four treatment regimens as described in Example 1. Specifically, within DLBCL, the following scenarios were simulated:
a. Scenario 1: Regimens r_{1,1} and r_{1,2} are safe and effective. Regimens r_{-1,1} and r_{-1,2} are ineffective
b. Scenario 2: Regimens r_{1,1} and r_{1,2} are unsafe. Regimens r_{-1,1} and r_{-1,2} are safe and effective
c. Scenario 3: Regimens r_{1,1}, r_{-1,1} and r_{-1,2} are ineffective. Regimen r_{1,2} is safe and effective
d. Scenario 4: All regimens either unsafe or ineffective

Within each of the 4 scenarios, the following 3 sub-scenarios describing the relationship between the efficacy in DLBCL and MCL were simulated.
a. 0.1: MCL is equal to that in DLBCL across all regimens
b. 0.2: Regimens are less effective in MCL than CLBCL (higher CR rates in DLBCL)
c. 0.3: Regimens are more effective in MCL than DLBCL (higher CR rates in MCL)

For each scenario, 1000 trials were simulated in silico to determine the following characteristics:
Probability that each regimen has been opened at some point in the trial
Average number of patients enrolled to each regimen
Power/Type I Error: Probability that the final highest open utility regimen, r_{d*,s*}, is likely effective and safe (Pr(θ_{d*,s*,h}>25%)>80% and Pr(π_{d*,s*}<33%)>50%)
Probability each regimen is selected as the final highest utility regimen and that regimen is likely effective and safe

3A: Scenario 1

For scenario 1, all regimens were designated to be safe for both disease cohorts, Dose level −1 was designated ineffective under both schedules, and Dose level 1 was designated effective under both schedules.

The simulation results showed that both DLBCL and MCL had similar mean sample sizes of approximately 13 subjects per regimen per disease cohort for Dose level 1 under both schedules. The simulation results also demonstrate that, on average, none or very few patients were assigned to the two ineffective regimens. Under both disease cohorts, the probability that the simulation would identify and select a final regimen that was likely effective and safe was 99%. Dose level 1, 2-dose schedule was selected correctly as the highest utility regimen with a probability of approximately 95%.

3B: Scenario 2

For this simulation, both schedules of dose level 1 were designated as unsafe and both schedules of Dose level −1 were designated both safe and effective.

The simulation results showed that Dose level −1 averaged the highest enrollment under both disease cohorts and both schedules. Fewer MCL patients were placed into unsafe regimens than DLBCL patients. One possible explanation for this discrepancy is that the study accrues DLBCL patients at a faster rate than MCL patients and safety data are pooled between disease cohorts. Thus, the simulation reaches a safety determination at a point when more DLBCL patients are in the trial than MCL patients. The results show that the probability that the simulation would identify the highest utility regimen correctly was 83%-85%. The probability that the trial would successfully find a safe and effective regimen was 93%.

3C: Scenario 3

For this simulation, all regimens were designated as safe and Dose level 1, 2-dose schedule is the only effective regimen.

The simulation results showed that the initial regimen and the highest utility regimen averaged the largest enrollment sizes. The probability that the simulation would identify dose level 1, 2-dose schedule, the highest utility regimen, correctly was 83-85%. The probability that the trial would successfully find a safe and effective regimen was 85-86%.

3D: Scenario 4

For this simulation, no regimens were designated as both safe and effective.

Dose level −1, 2-dose schedule was defined to have a 21% DLT rate and a 25% CR rate, which under the parameters of this trial was safe, but not efficacious. All of the other regimens had lower CR rates.

The simulation results showed that most patients were allocated to the Dose level −1, 2-dose schedule for both the DLBCL and MCL cohorts. As with Example 3B, fewer MCL patients were placed into the unsafe regimens. The results show that the probability that the simulation would identify that no regimens are both safe and effective was 82%. The dose level −1, 2-dose schedule was selected most often when the trial incorrectly declared success at finding a safe and effective regimen (16% probability).

3E: Summary

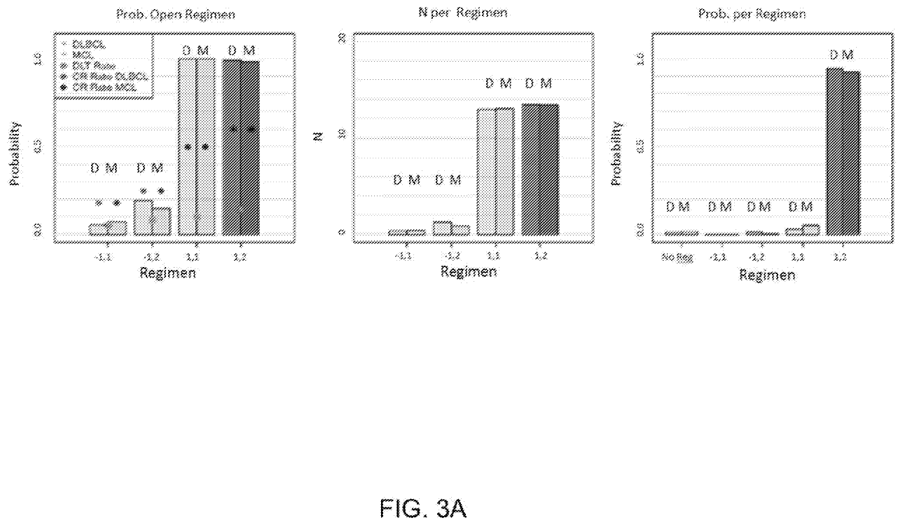

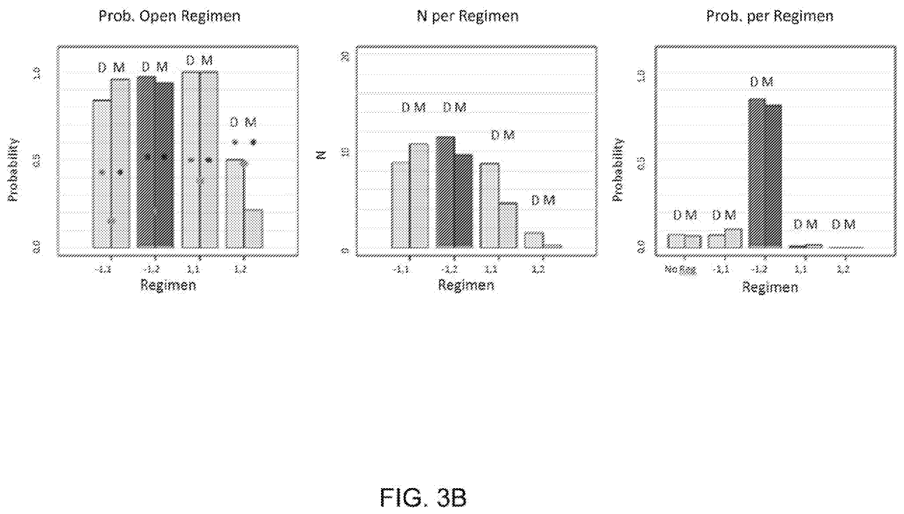

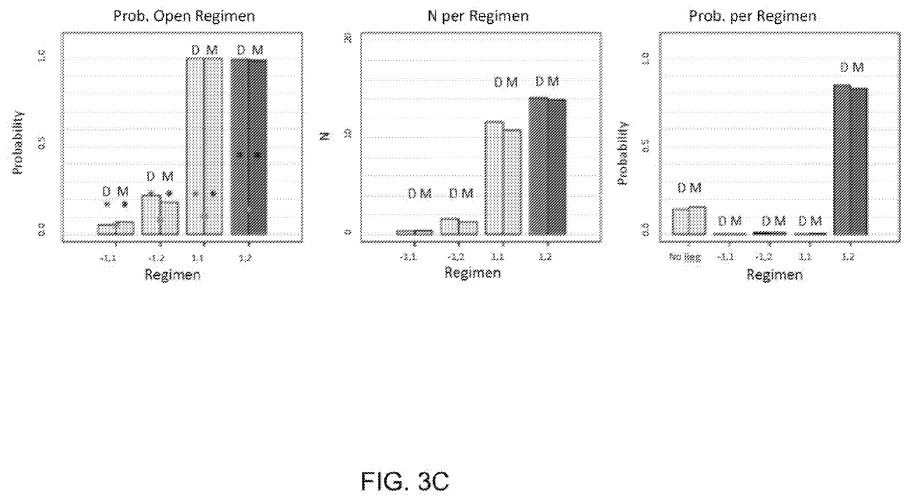

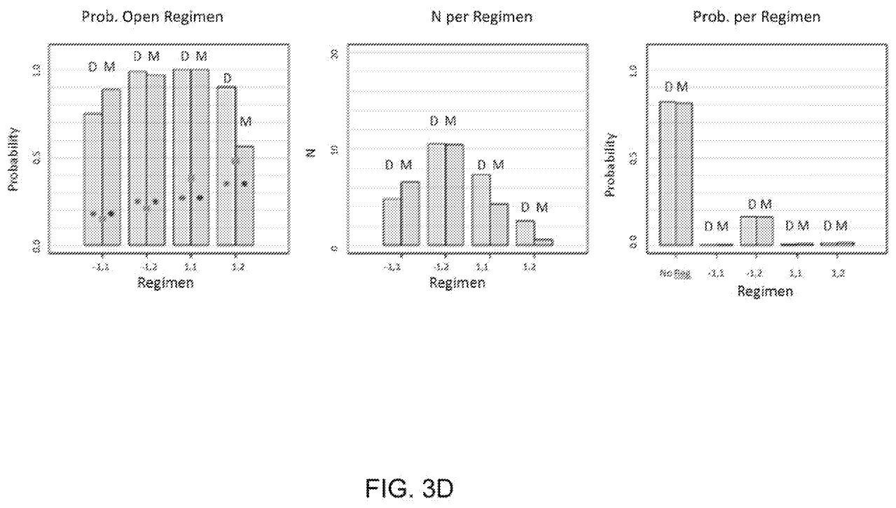

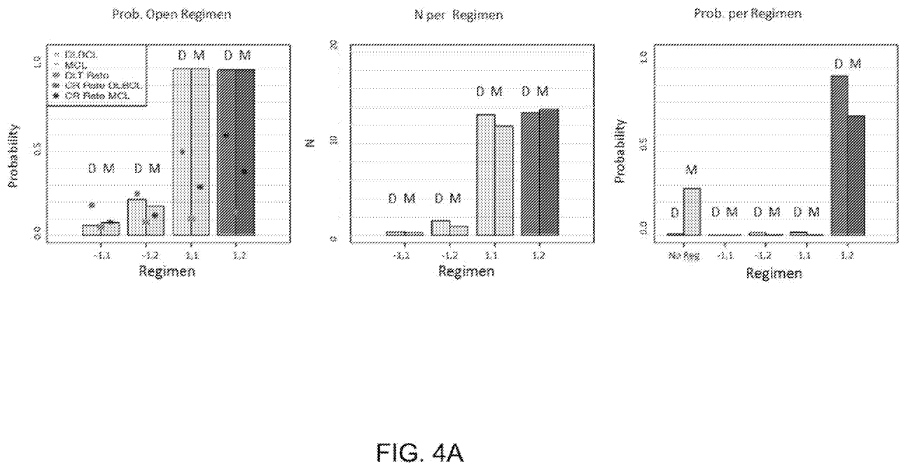

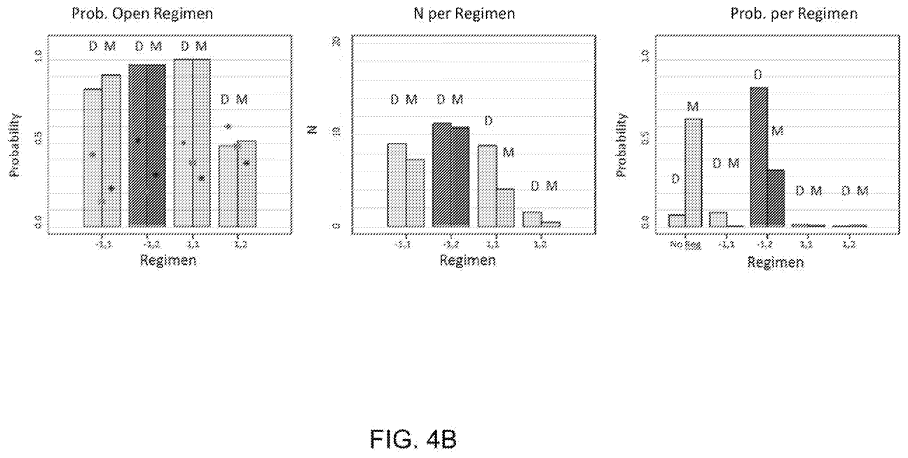

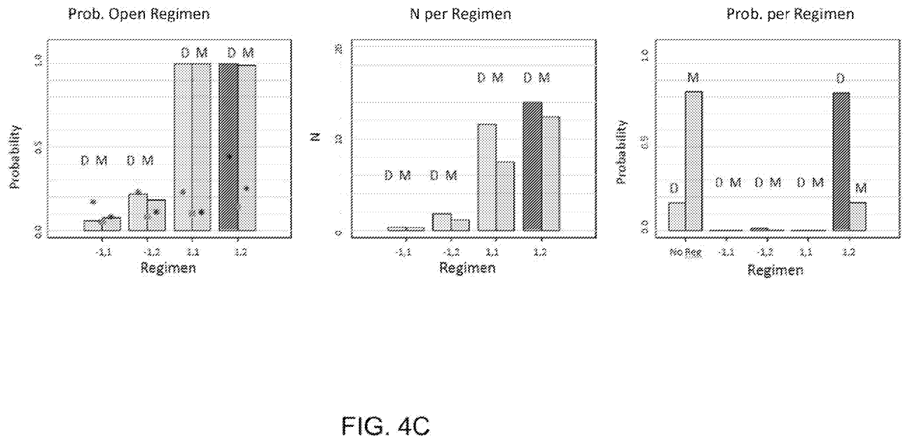

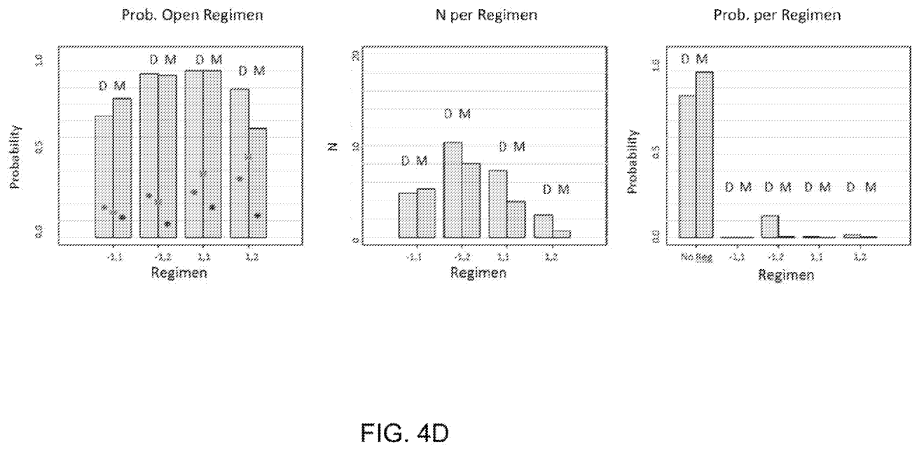

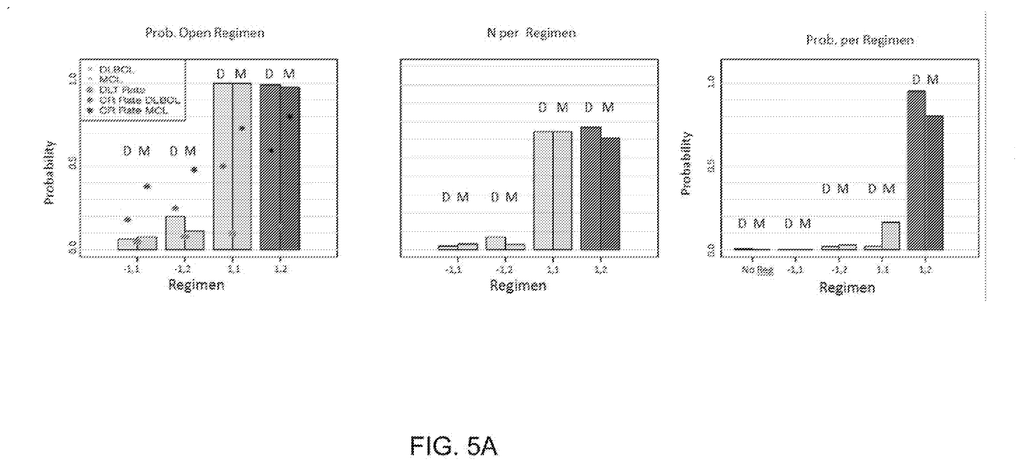

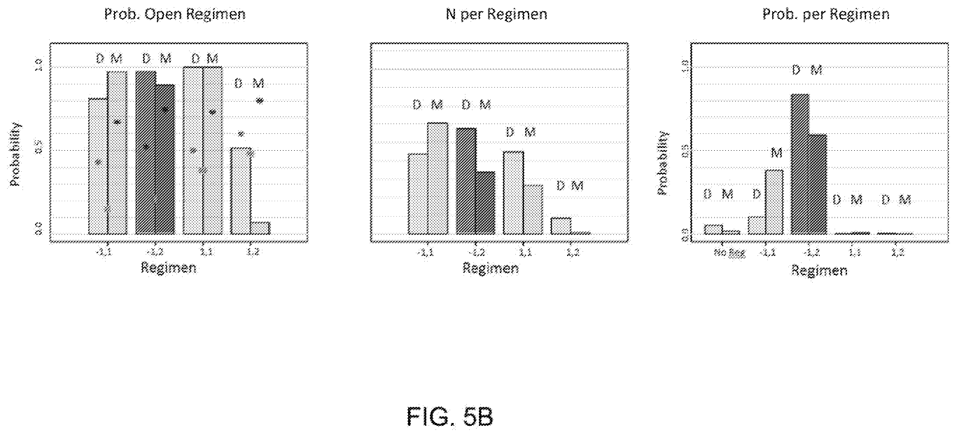

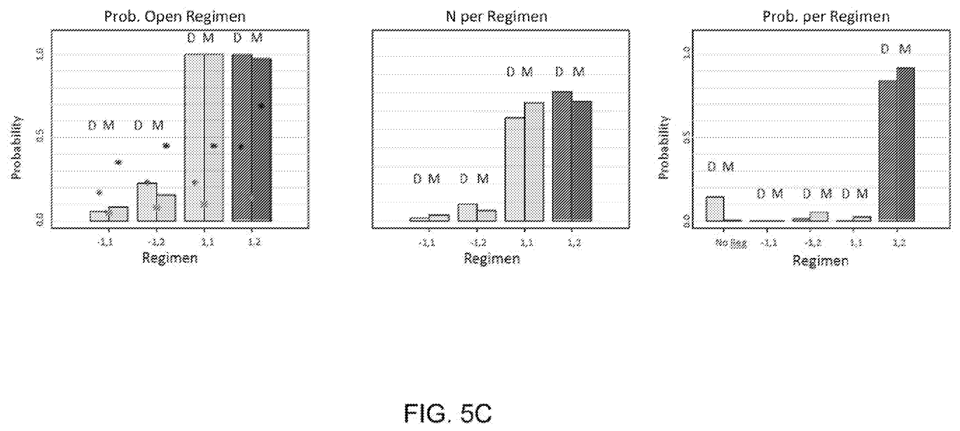

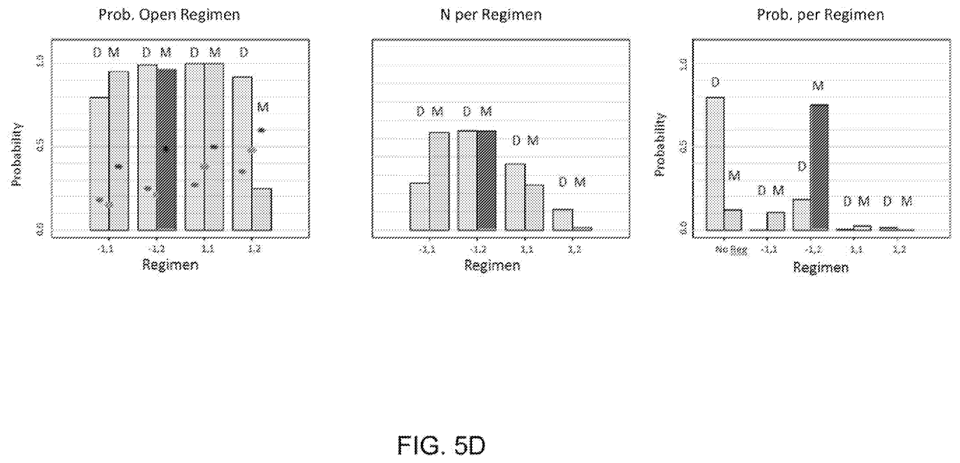

Tables 7A-B (DLBCL) and Tables 8A-B (MCL) summarize the operating characteristics under the various sub-scenarios. Tables 9A-B (DLBCL) and Tables 10A-B and FIGS. 3-5 summarize the operating characteristics under the various sub-scenarios from updated simulations. FIG. 3 summarizes scenarios 1.1, 2.1, 3.1, and 4.1 when the CR rates of the MCL and DLBCL disease cohorts are equal for each regimen. FIGS. 4 and 5 summarize scenarios 1.2, 2.2, 3.2, and 4.2, and 1.3, 2.3, 3.3, and 4.3 respectively (when the regimens are less effective in MCL than DLBCL for FIG. 4 and when the regimens are more effective in MCL than DLBCL for FIG. 5).

TABLE 7A
Operating Characteristics Within DLBCL

| | Probability regimen opens | | | | N per regimen | | | |
|---|---|---|---|---|---|---|---|---|
| Scenario | r-1, 1 | r-1, 2 | r 1, 1 | r 1, 2 | r-1, 1 | r-1, 2 | r 1, 1 | r 1, 2 |
| 1.1 | 0.06 | 0.17 | 1.00 | 0.99 | 0 | 1 | 13 | 13 |
| 1.2 | 0.06 | 0.22 | 1.00 | 1.00 | 0 | 2 | 13 | 13 |
| 1.3 | 0.08 | 0.20 | 1.00 | 1.00 | 0 | 1 | 13 | 13 |
| 2.1 | 0.79 | 0.97 | 1.00 | 0.56 | 8 | 11 | 9 | 2 |
| 2.2 | 0.82 | 0.97 | 1.00 | 0.50 | 9 | 11 | 9 | 1 |
| 2.3 | 0.80 | 0.97 | 1.00 | 0.50 | 8 | 11 | 9 | 2 |
| 3.1 | 0.05 | 0.20 | 1.00 | 1.00 | 0 | 2 | 11 | 14 |
| 3.2 | 0.06 | 0.21 | 1.00 | 1.00 | 0 | 2 | 12 | 14 |
| 3.3 | 0.05 | 0.20 | 1.00 | 1.00 | 0 | 2 | 11 | 14 |
| 4.1 | 0.75 | 0.99 | 1.00 | 0.89 | 5 | 11 | 7 | 2 |
| 4.2 | 0.74 | 0.98 | 1.00 | 0.88 | 5 | 10 | 7 | 2 |
| 4.3 | 0.75 | 0.99 | 1.00 | 0.93 | 5 | 11 | 7 | 2 |

TABLE 7B
Operating Characteristics Within DLBCL

| | Probability of being selected | | | | | Trial Size | | |
|---|---|---|---|---|---|---|---|---|
| | as highest open utility regimen | | | | Probability | Prob. | | |
| Scenario | r −1, 1 | r −1, 2 | r 1, 1 | r 1, 2 | of success | Max | Mean | 90th % |
| 1.1 | 0 | 0.02 | [symbol] | [symbol] | 0.99 | 0.11 | 28 | 35 |
| 1.2 | 0 | 0.02 | [symbol] | [symbol] | 0.99 | 0.12 | 28 | 35 |
| 1.3 | 0 | 0.02 | 0.03 | 0.95 | 0.99 | 0.1 | 28 | 35 |
| 2.1 | [symbol] | [symbol] | 0.01 | 0.01 | 0.92 | 0.43 | 30 | 35 |
| 2.2 | [symbol] | 0.87 | 0.01 | 0.01 | 0.91 | 0.41 | 30 | 35 |
| 2.3 | 0.12 | [symbol] | 0.01 | 0.01 | 0.93 | 0.38 | 30 | 35 |
| 3.1 | 0 | 0.03 | 0 | [symbol] | 0.84 | 0.13 | 27 | 35 |
| 3.2 | 0 | 0.02 | 0 | [symbol] | 0.83 | 0.17 | 28 | 35 |
| 3.3 | 0 | 0.03 | 0 | 0.97 | 0.87 | 0.15 | 27 | 35 |
| 4.1 | 0.01 | 0.79 | 0.02 | 0.17 | 0.16 | 0.18 | 25 | 35 |
| 4.2 | 0.02 | 0.73 | 0.03 | 0.22 | 0.14 | 0.17 | 24 | 35 |
| 4.3 | 0.01 | 0.85 | 0.01 | 0.13 | 0.22 | 0.19 | 25 | 35 |

TABLE 8A
Operating Characteristics Within MCL

| | Probability regimen opens | | | | N per regimen | | | |
|---|---|---|---|---|---|---|---|---|
| Scenario | r-1, 1 | r-1, 2 | r 1, 1 | r 1, 2 | r-1, 1 | r-1, 2 | r 1, 1 | r 1, 2 |
| 1.1 | 0.06 | 0.13 | 1.00 | 0.98 | 0 | 1 | 13 | 13 |
| 1.2 | 0.07 | 0.18 | 1.00 | 1.00 | 0 | 1 | 12 | 14 |
| 1.3 | 0.09 | 0.13 | 1.00 | 0.97 | 1 | 1 | 13 | 12 |
| 2.1 | 0.94 | 0.93 | 1.00 | 0.23 | 10 | 9 | 5 | 0 |
| 2.2 | 0.90 | 0.97 | 1.00 | 0.52 | 7 | 11 | 4 | 0 |
| 2.3 | 0.97 | 0.89 | 1.00 | 0.07 | 12 | 7 | 5 | 0 |
| 3.1 | 0.06 | 0.16 | 1.00 | 0.99 | 0 | 1 | 11 | 14 |
| 3.2 | 0.07 | 0.18 | 1.00 | 1.00 | 0 | 1 | 8 | 13 |
| 3.3 | 0.07 | 0.14 | 1.00 | 0.99 | 0 | 1 | 13 | 13 |
| 4.1 | 0.89 | 0.97 | 1.00 | 0.55 | 6 | 11 | 4 | 1 |
| 4.2 | 0.86 | 0.97 | 1.00 | 0.60 | 5 | 8 | 4 | 1 |
| 4.3 | 0.94 | 0.96 | 1.00 | 0.27 | 10 | 11 | 5 | 0 |

TABLE 8B
Operating Characteristics Within MCL

| | Probability of being selected | | | | | | | |
|---|---|---|---|---|---|---|---|---|
| | as highest open utility regimen | | | | Probability | Trial Size | | |
| Scenario | r −1, 1 | r −1, 2 | r 1, 1 | r 1, 2 | of success | Prob. Max | Mean | 90th % |
| 1.1 | 0.00 | 0.02 | [symbol] | [symbol] | 0.99 | 0.01 | 28 | 31 |
| 1.2 | 0.00 | 0.01 | 0.01 | [symbol] | 0.69 | 0.03 | 27 | 33 |
| 1.3 | [symbol] | [symbol] | 0.17 | 0.80 | 1.00 | 0.01 | 27 | 30 |
| 2.1 | [symbol] | [symbol] | 0.02 | 0.00 | 0.94 | 0.03 | 25 | 31 |
| 2.2 | 0.05 | [symbol] | 0.01 | 0.04 | 0.35 | 0.02 | 22 | 30 |
| 2.3 | [symbol] | [symbol] | 0.01 | 0.00 | 0.98 | 0.03 | 24 | 30 |
| 3.1 | 0.00 | 0.03 | 0.01 | [symbol] | 0.85 | 0.04 | 27 | 33 |
| 3.2 | 0.00 | 0.02 | 0.00 | 0.98 | 0.17 | 0.05 | 22 | 32 |
| 3.3 | 0.00 | 0.04 | [symbol] | [symbol] | 0.99 | 0.02 | 28 | 31 |
| 4.1 | 0.03 | 0.82 | 0.04 | 0.12 | 0.18 | 0.07 | 22 | 32 |
| 4.2 | 0.03 | 0.67 | 0.05 | 0.26 | 0.01 | 0.00 | 18 | 25 |
| 4.3 | [symbol] | [symbol] | 0.02 | 0.01 | 0.87 | 0.10 | 27 | 35 |

TABLE 9A
Operating Characteristics Within DLBCL

| | Probability regimen opens | | | | N per regimen | | | |
|---|---|---|---|---|---|---|---|---|
| Scenario | r-1, 1 | r-1, 2 | r 1, 1 | r 1, 2 | r-1, 1 | r-1, 2 | r 1, 1 | r 1, 2 |
| 1.1 | 0.06 | 0.19 | 1.00 | 0.99 | 0 | 1 | 13 | 13 |
| 1.2 | 0.06 | 0.21 | 1.00 | 0.99 | 0 | 2 | 13 | 13 |
| 1.3 | 0.06 | 0.20 | 1.00 | 0.99 | 0 | 1 | 13 | 13 |
| 2.1 | 0.84 | 0.97 | 1.00 | 0.50 | 9 | 11 | 9 | 2 |
| 2.2 | 0.82 | 0.97 | 1.00 | 0.48 | 9 | 11 | 9 | 2 |
| 2.3 | 0.81 | 0.98 | 1.00 | 0.52 | 9 | 11 | 9 | 2 |
| 3.1 | 0.05 | 0.22 | 1.00 | 1.00 | 0 | 2 | 12 | 14 |
| 3.2 | 0.06 | 0.22 | 1.00 | 1.00 | 0 | 2 | 12 | 14 |
| 3.3 | 0.06 | 0.22 | 1.00 | 1.00 | 0 | 2 | 11 | 14 |
| 4.1 | 0.75 | 0.99 | 1.00 | 0.90 | 5 | 11 | 7 | 3 |
| 4.2 | 0.73 | 0.98 | 1.00 | 0.89 | 5 | 10 | 7 | 2 |
| 4.3 | 0.80 | 0.99 | 1.00 | 0.92 | 5 | 11 | 7 | 2 |

TABLE 9B
Operating Characteristics Within DLBCL

| | Probability of being | | | | Prob- | | | |
|---|---|---|---|---|---|---|---|---|
| | selected as highest | | | | ability | Trial Size | | |
| | open utility regimen | | | | of | Prob. | | |
| Scenario | r-1, 1 | r-1, 2 | r 1, 1 | r 1, 2 | success | Max | Mean | 90th % |
| 1.1 | 0 | 0.01 | 0.03 | 0.94 | 0.99 | 0.10 | 28 | 34 |
| 1.2 | 0 | 0.02 | 0.02 | 0.96 | 0.99 | 0.13 | 29 | 35 |
| 1.3 | 0 | 0.02 | 0.02 | 0.95 | 0.99 | 0.11 | 28 | 35 |
| 2.1 | 0.07 | 0.84 | 0.01 | 0 | 0.93 | 0.40 | 30 | 35 |
| 2.2 | 0.08 | 0.83 | 0.01 | 0 | 0.93 | 0.41 | 31 | 35 |
| 2.3 | 0.10 | 0.84 | 0 | 0.01 | 0.95 | 0.41 | 31 | 35 |
| 3.1 | 0 | 0.01 | 0 | 0.85 | 0.86 | 0.16 | 28 | 35 |
| 3.2 | 0 | 0.01 | 0 | 0.83 | 0.84 | 0.18 | 28 | 35 |
| 3.3 | 0 | 0.01 | 0 | 0.84 | 0.86 | 0.15 | 28 | 35 |
| 4.1 | 0 | 0.16 | 0.01 | 0.01 | 0.18 | 0.18 | 25 | 35 |
| 4.2 | 0 | 0.13 | 0.01 | 0.02 | 0.15 | 0.19 | 25 | 35 |
| 4.3 | 0 | 0.18 | 0.01 | 0.01 | 0.20 | 0.22 | 25 | 35 |

TABLE 10A
Operating Characteristics Within MCL

| | Probability regimen opens | | | | N per regimen | | | |
|---|---|---|---|---|---|---|---|---|
| Scenario | r-1, 1 | r-1, 2 | r 1, 1 | r 1, 2 | r-1, 1 | r-1, 2 | r 1, 1 | r 1, 2 |
| 1.1 | 0.07 | 0.15 | 1.00 | 0.98 | 0 | 1 | 13 | 13 |
| 1.2 | 0.07 | 0.18 | 1.00 | 0.99 | 0 | 1 | 12 | 14 |
| 1.3 | 0.08 | 0.11 | 1.00 | 0.98 | 1 | 1 | 13 | 12 |
| 2.1 | 0.96 | 0.94 | 1.00 | 0.21 | 11 | 10 | 5 | 0 |
| 2.2 | 0.91 | 0.97 | 1.00 | 0.51 | 7 | 11 | 4 | 0 |
| 2.3 | 0.97 | 0.89 | 1.00 | 0.07 | 12 | 7 | 5 | 0 |
| 3.1 | 0.07 | 0.18 | 1.00 | 0.99 | 0 | 1 | 11 | 14 |
| 3.2 | 0.08 | 0.18 | 1.00 | 0.99 | 0 | 1 | 7 | 12 |
| 3.3 | 0.08 | 0.16 | 1.00 | 0.97 | 1 | 1 | 13 | 13 |
| 4.1 | 0.89 | 0.97 | 1.00 | 0.56 | 7 | 10 | 4 | 1 |
| 4.2 | 0.83 | 0.97 | 1.00 | 0.65 | 5 | 8 | 4 | 1 |
| 4.3 | 0.95 | 0.96 | 1.00 | 0.27 | 11 | 11 | 5 | 0 |

TABLE 10B
Operating Characteristics Within MCL

| | Probability of being | | | | Prob- | | | |
|---|---|---|---|---|---|---|---|---|
| | selected as highest | | | | ability | Trial Size | | |
| | open utility regimen | | | | of | Prob. | | |
| Scenario | r-1, 1 | r-1, 2 | r 1, 1 | r 1.2 | success | Max | Mean | 90th % |
| 1.1 | 0.00 | 0.01 | 0.05 | 0.93 | 0.99 | 0.01 | 28 | 31 |
| 1.2 | 0.00 | 0.00 | 0.00 | 0.72 | 0.72 | 0.03 | 27 | 33 |
| 1.3 | 0.00 | 0.03 | 0.17 | 0.80 | 1.00 | 0.00 | 26 | 30 |
| 2.1 | 0.11 | 0.81 | 0.02 | 0.00 | 0.94 | 0.03 | 25 | 31 |
| 2.2 | 0.01 | 0.34 | 0.01 | 0.01 | 0.36 | 0.02 | 23 | 30 |
| 2.3 | 0.38 | 0.59 | 0.01 | 0.00 | 0.98 | 0.02 | 24 | 30 |
| 3.1 | 0.00 | 0.01 | 0.01 | 0.83 | 0.85 | 0.05 | 26 | 32 |
| 3.2 | 0.00 | 0.00 | 0.00 | 0.17 | 0.17 | 0.05 | 21 | 31 |
| 3.3 | 0.00 | 0.05 | 0.02 | 0.92 | 1.00 | 0.03 | 28 | 31 |
| 4.1 | 0.00 | 0.16 | 0.01 | 0.02 | 0.19 | 0.07 | 22 | 32 |
| 4.2 | 0.00 | 0.00 | 0.00 | 0.00 | 0.01 | 0.00 | 18 | 24 |
| 4.3 | 0.11 | 0.75 | 0.02 | 0.00 | 0.88 | 0.12 | 27 | 35 |

For this simulation, the sub-scenarios assumed that the highest utility regimen was different between DLBCL and MCL patients. Under sub-scenario 3.2 within DLBCL, Dose level 1, 2-dose schedule was designated as the only safe and effective regimen for DLBCL (DLT rate of 14% and CR rate of 45%). No regimens were designated as safe and effective for MCL.

The simulation results showed that the overall probability of incorrectly claiming success in identifying a safe and effective regimen within MCL was 17% with the most likely selected regimen being Dose level 1, 2-dose schedule (CR rate of 25% and DLT rate of 14%).

Under sub-scenario 4.3 within DLBCL, no regimens were designated as safe and effective and within MCL dose level −1 under both dose schedules was both safe and effective (18% and 25% DLT rates and 38% and 48% CR rates, respectively). This simulation produced an overall probability of correctly claiming success in identifying a safe and effective regimen within MCL of 88%. The simulation produced a probability of incorrectly claiming success in identifying a safe and effective regimen within DLBCL of 20%.

The ability to discern a difference in DLBCL and MCL within sub-scenarios 3.2 and 4.3 demonstrates that there is not too much borrowing of information across disease cohorts.

### Example 4 Comparison with 3+3 Design

The clinical trial design described in Example 1 was compared to the 3+3 method. A total of 1000 simulations for each method were generated. The simulation parameters assumed the CR rate was the same between two diseases. The 2 sets of assumed DLT rates in Table 5 were used in the simulation. Both 3+3L and 3+3H were evaluated with the MTD defined as the highest dose with ≤1 and ≤2 DLTs among 6 patients, respectively. The percentage of simulated trials selecting the true MTD are shown in Table 8.

TABLE 8
3 + 3 Design Simulation

| | | | Probability of | Probability of |
|---|---|---|---|---|
| | | | Selecting True | Selecting True |
| Scenario | Regimen | Pr(DLT) | MTD (3 + 3^{L}) | MTD (3 + 3^{H}) |
| 1 | r_{1, 1} | 0.10 | 0.15 | 0.12 |
| | r_{1, 2} | 0.14 | 0.75 | 0.85 |
| | r_{-1, 1} | 0.05 | 0.01 | 0.00 |
| | r_{-1, 2} | 0.08 | 0.09 | 0.03 |
| 2 | r_{1, 1} | 0.38 | 0.22 | 0.40 |
| | r_{1, 2} | 0.48 | 0.06 | 0.12 |
| | r_{-1, 1} | 0.15 | 0.25 | 0.08 |
| | r_{-1, 2} | 0.21 | 0.46 | 0.39 |

The results show the trial using the Bayesian adaptive design is more reliable and safer than the 3+3 trial design. The percentage of simulated Bayesian adaptive design trials that selected highest open utility regimen in Table 6 and 7 is higher than the percentage of simulated 3+3 trials that selected the true MTD in Table 8.

In scenario 1, in which all doses were designated safe, the Bayesian adaptive design had 0.92-0.97 probability of selecting the regimen with highest utility, r1,2, while the 3+3 design correctly selected the r1,2 with only 0.75 or 0.85 probability.

In scenario 2, dose level 1 is unsafe in both schedules. The Bayesian adaptive design had 0.80-0.86 probability of selecting the highest acceptable regimen, r−1,2, while the 3+3 design had only 0.4 or 0.46 probability. More importantly, 3+3 design had a probability of selecting an unsafe dose level 1 (either single-dose or 2-dose schedule) as the MTD of 0.28 or 0.52, while the Bayesian adaptive design had only a probability of <0.05-0.19 of selecting an unsafe dose level 1 regimen as the MTD, which depended on the assumed CR rates.

Herein, a Bayesian adaptive design to address the challenges and practical aspects of enrolling a trial with multiple dosing regimens and multiple disease subtypes is presented. The proposed design is flexible and demonstrates good operating characteristics through simulation studies of diverse clinical scenarios. Although the design of the Examples is tested in the setting of a mixed population, the regimen-efficacy model can be easily modified and applied to only one homogeneous population. Simulation studies for this scenario were performed and demonstrate that the design still performs well for this scenario.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-125. (canceled)

126. A method for treatment of a subject with a therapeutic agent, comprising:
a) designating two or more unique treatment regimens for administering a therapeutic agent;
b) calculating an overall utility score for at least one treatment regimen, wherein the overall utility score is based on response information of, and on toxicity information of, one or more subjects previously treated with the therapeutic agent according to the treatment regimen;
c) allocating a subject to a selected treatment regimen based on the overall utility score; and
d) administering to the subject the therapeutic agent according to the selected treatment regimen in which the subject has been allocated.

127. A method for treatment of a subject with a therapeutic agent, comprising administering to a subject a therapeutic agent according to a selected treatment regimen,
wherein the selected treatment regimen is selected from two or more possible unique treatment regimens for administering the therapeutic agent, based on an overall utility score that is calculated for at least one treatment regimen, wherein the overall utility score for each of the at least one treatment regimen is based on response information of subjects of, and on toxicity information of, one or more subjects previously treated with the therapeutic agent according to the treatment regimen.

128. The method of claim 126, wherein the calculating of the overall utility score for at least one treatment regimen comprises calculating the overall utility score for each of the two or more unique treatment regimens for administering the therapeutic agent.

129. The method of claim 126, wherein the one or more subjects are enrolled in a clinical trial and/or wherein the previous treatment of the one or more subjects is carried out in a single clinical trial and/or the subject to whom the selected treatment regimen is allocated is enrolled in a clinical trial and/or wherein the subject to whom the selected treatment regimen is allocated and the one or more subjects are enrolled in the same clinical trial.

130. The method of claim 126, wherein the allocation of the subject comprises random allocation of the subject to the selected treatment regimen based on a randomization probability, wherein a regimen with a higher utility score has a greater randomization probability than a regimen with lower utility score and/or wherein height of utility score positively influences the randomization probability.

131. The method of claim 126, wherein allocating the subject to the selected treatment regimen is further based on the number of subjects already allocated to one or more of the two or more treatment regimens or the number of open spots in each of the two or more treatment regimens.

132. The method of claim 126, wherein the one or more subjects comprises at least one subject in each of two or more disease cohorts.

133. The method of claim 132, wherein the calculating the overall utility score for said at least one treatment regimen comprises, for each of said at least one treatment regimen, calculating a separate overall disease-specific utility score for each of the two or more of disease cohorts for said treatment regimen.

134. The method of claim 132, wherein:
each of said two or more disease cohorts comprises subjects with one of a plurality of different diseases, one of a plurality of different grades of disease, one of a plurality of disease burden levels, one of a plurality of states of genetic mutation associated with the disease or condition and/or the treatment regimen, one of a plurality of tumor locations, one of a plurality of age groups of subjects with a disease, one of a gender of subjects with a disease, one of a plurality of weight groups of subjects with a disease, and/or one of a plurality of different number and/or types of prior therapies or treatments for the disease; or
subjects with different subtypes of the same disease are grouped into different disease cohorts.

135. The method of claim 133, wherein the overall disease-specific utility score is based on response information of subjects within the disease cohort treated with the therapeutic agent according to the treatment regimen and on toxicity information for subjects across a plurality of disease cohorts treated with the therapeutic agent according to the treatment regimen.

136. The method of claim 126, wherein the two or more unique treatment regimens differ in a) dose level of the therapeutic agent, b) a schedule of administration of the therapeutic agent; or (c) dose level of the therapeutic agent and a schedule of administration of the therapeutic agent.

137. The method of claim 133, wherein calculating the overall disease-specific utility score for a given disease cohort in each of the at least one treatment regimen comprises:
i) generating a safety utility score, wherein said safety utility score is based on a toxicity rate for the treatment regimen;
ii) generating an efficacy utility score for the treatment regimen, wherein said efficacy utility score is based on an efficacy rate for the treatment regimen; and
iii) multiplying the safety utility score by the efficacy utility score, thereby calculating the overall disease-specific utility score for the treatment regimen for the disease cohort.

138. The method of claim 137, wherein prior to (i)-(iii), further comprising a) determining the toxicity rate based on the toxicity information of a population of subjects treated according to the treatment regimen; and b) determining the efficacy rate based on response information of a population of subjects treated according to the treatment regimen.

139. The method of claim 138, wherein:
the population of subjects from which the toxicity information is determined comprises subjects in the disease cohort and subjects not in the disease cohort; and/or
the population of subjects from which the efficacy rate is determined comprises subjects in the disease cohort and does not comprise subjects not in the disease cohort.

140. The method of claim 137, wherein:
the safety utility score is based on toxicity information from subjects in the disease cohort and from subjects not in the disease cohort; and/or
the efficacy utility score is based on response information from subjects in the disease cohort and not from subjects not in the disease cohort.

141. The method of claim 137, wherein:
(A) in step i):
if the toxicity rate is determined to be less than or equal to a utility safety target, then the function defines the safety utility score as 1;
if the toxicity rate is determined to be greater or equal to a utility safety limit, then the function defines the safety utility score as 0; and
if the toxicity rate is projected to be between the utility safety target and the utility safety limit, then the safety utility score decreases linearly as the toxicity rate increases; and/or
(B) in step ii):
if the efficacy rate is projected to be less than or equal to a utility efficacy target, then the function defines the efficacy utility score as 0;
if the efficacy rate is projected to be greater than the utility efficacy target, then the efficacy utility score increases linearly as the efficacy rate increases.

142. The method of claim 141, wherein:
a) the safety utility score is set as zero when the toxicity rate is above a 0.33, and the safety utility score is set at one when the toxicity rate is below a 0.2, and wherein the safety utility score decreases in a linear manner from one to zero when the toxicity rate increases from 0.20 to 0.33; and/or
b) the efficacy utility score is set as zero when the efficacy rate is below 0.25, and the efficacy utility score is set at one when the efficacy rate is 1, and wherein the efficacy utility score increases in a linear manner from zero to one when the efficacy rate increases from 0.25 to 1.

143. The method of claim 137, wherein the toxicity rate is a dose-limiting toxicity (DLT) rate.

144. The method of claim 137, wherein the efficacy rate is a complete response (CR) rate.

145. The method of claim 137, wherein the toxicity rate is determined by a regimen-toxicity model that estimates a single toxicity rate for each regimen across all disease cohorts.

146. The method of claim 145, wherein the regimen-toxicity model comprises the formula:
$$log ( \frac{π}{1 - π} ) = {β}_{0} + {β}_{1} {X}_{1} … + {β}_{n} {X}_{n}$$
wherein π is the DLT rate, wherein β₀ is a first regimen that is set as the referent, wherein β₁ X₁ . . . βₙXₙ are the additive effects of the remaining regimens in relation to the first regimen, and wherein n is equal to the number of regimens minus one.

147. The method of claim 146, wherein the regimen-toxicity model comprises the formula:
$$log ( \frac{{π}_{d , s}}{1 - {π}_{d , s}} ) = {β}_{0} + {β}_{1} {1}_{[ d = - 1 ]} + {β}_{2} {1}_{[ s = 2 ⋂ d = 1 ]} + {B}_{3} {1}_{[ s = 2 ⋂ d = - 1 ]}$$

148. The method of claim 137, wherein the efficacy rate is determined by a regimen-response model that estimates a single efficacy rate within each disease cohort for each regimen.

149. The method of claim 148, wherein the regimen-efficacy model comprises the formula:
$$log ( \frac{θ}{1 - θ} ) = {α}_{0} + {α}_{1} {X}_{1} … + {α}_{n} {X}_{n}$$
wherein θ is the CR rate, wherein α₀ is a first regimen that is set as the referent, wherein α₁ X₁ . . . αₙXₙ are the additive effects of the remaining regimens in relation to the first regimen, and wherein is n is equal to the number of regimens minus one.

150. The method of claim 149, wherein the regimen response model comprises the formula:
$$log ( \frac{{θ}_{d , s , h}}{1 - {θ}_{d , s , h}} ) = {α}_{0 , h} + {α}_{1} {1}_{[ d = - 1 ]} + {α}_{2} {1}_{[ s = 2 ⋂ d = 1 ]} + {α}_{3} {1}_{[ s = 2 ⋂ d = - 1 ]}$$

151. The method of claim 148, wherein the response information is borrowed across disease cohorts according to the following distribution:
$${α}_{0 cohort 1} , {α}_{0 cohort 2} N ( θ , {σ}^{2} ) ; θ \sim N ( - 0.5 , {4}^{2} ) ; \frac{1}{{σ}^{2}} \sim Gamma ( 2 , 2 ) .$$

152. The method of claim 126, wherein:
each of the two or more treatment regimens has a first status of either open or closed; and
the subject is allocated into an open treatment regimen.

153. The method of claim 152, wherein prior to allocating the subject, the method further comprises:
determining if the first status of any of the two or more treatment regimens should be changed from closed to open; and
if the first status of a treatment regimen should be changed, changing the first status to open.

154. The method of claim 126, wherein allocating the subject to a selected treatment regimen is further based on the relative uncertainty of the estimate for the overall utility score for each of the at least two or more possible unique treatment regimen.

155. The method of claim 154, wherein allocating a subject to a selected treatment regimen is based on a randomization probability that a subject will be enrolled in each of the regimens with a first status of open and a second status of eligible, wherein the randomization probability Vₓ is:
$${V}_{x} ∝ {[ \frac{Pr ( {r}_{x} = {r}_{{x}^{*}} ) Var ( {U}_{x} )}{{n}_{x} + 1} ]}^{\frac{1}{2}} .$$
wherein rₓ is the regimen x, Pr(rₓ=r_{x*}) is the probability the regimen x is the highest utility regimen, Var(Uₓ) is the variance of the regimen's utility score, and nₓ is the number of subjects already allocated to the regimen.

156. The method of claim 154, wherein allocating a subject to a selected treatment regimen is based on a randomization probability that a subject will be enrolled in each of the regimen within each disease cohort with a first status of open and a second status of eligible, wherein the randomization probability V_{d,s,h} is:
$${V}_{d , s , h} ∝ {[ \frac{Pr ( {r}_{d , s , h} = {r}_{{d}^{*} , {s}^{*} , h} ) Var ( {U}_{d , s , h} )}{{n}_{d , s , h} + 1} ]}^{\frac{1}{2}} .$$
wherein r_{d,s,h} is the regimen of dose level d, schedules, and disease h, Pr(r_{d,s,h}=r_{d*,s*,h}) is the probability the regimen is the highest utility regimen, Var(U_{d,s,h}) is the variance of the regimen's disease-specific utility score, and n_{d,s,h} is the number of subjects already allocated to the regimen within disease cohort h.

157. The method of claim 126, wherein the therapeutic agent is one in which the response information can be determined within the same period of time in which the toxicity information is determined.

158. The method of claim 157, wherein the period of time is within about 28 days starting from when the first dose of the therapeutic agent is administered in the regimen; or wherein the period of time begins when the first dose of the therapeutic agent is administered in the regimen and extends for up to about 28 days after the last dose of the therapeutic agent is administered in the regimen.

159. The method of claim 126, wherein the therapeutic agent comprises an adoptive cell therapy, a small molecule, a gene therapy, or a transplant.

160. The method of claim 126, wherein the therapeutic agent comprises cells expressing a chimeric antigen receptor (CAR).

161. The method of claim 132, wherein at least one of the diseases is a tumor or a cancer.

162. A method for allocating a subject to a selected treatment regimen for administering a therapeutic agent, comprising:
a) calculating an overall utility score for at least one of a plurality of two or more unique treatment regimens for administering a therapeutic agent, wherein, for each of the at least one treatment regimens, the overall utility score is based on response information of, and on toxicity information of, one or more subjects previously treated with the therapeutic agent according to the treatment regimen; and
b) allocating the subject to a selected treatment regimen based on the overall utility score.

163. The method of claim 162, further comprising designating the two or more unique treatment regimens prior to step a).

164. The method of claim 162, further comprising treating the subject that has been allocated to a selected treatment regimen with the selected treatment regimen.

165. A method of calculating an overall disease-specific utility score for each disease cohort in a treatment regimen, comprising:
a) determining a toxicity rate based on the toxicity information of subjects previously treated with a therapeutic agent according to a treatment regimen;
b) determining an efficacy rate based on the response information of subjects previously treated with the therapeutic agent according to the treatment regimen;
c) generating a safety utility score as a function of the toxicity rate for the treatment regimen;
d) generating an efficacy utility score as a function of the efficacy rate for the treatment regimen; and
e) multiplying the safety utility score by the efficacy utility score, thereby calculating the overall utility score for the treatment regimen.

166. A computer system comprising a processor and memory, the memory comprising instructions operable to cause the processor to carry out any one or more of steps a)-c) of the method of claim 126.

167. A computer system comprising a processor and memory, the memory comprising instructions operable to cause the processor to carry out any one or more of steps of method of claim 127.

168. A computer system comprising a processor and memory, the memory comprising instructions operable to cause the processor to carry out any one or more of steps a)-b) of the method of claim 162.

169. A computer system comprising a processor and memory, the memory comprising instructions operable to cause the processor to carry out any one or more of steps a)-e) of the method of claim 165.

170. A method for identifying an optimal treatment regimen in a clinical trial, comprising:
defining two or more different treatment regimens;
assigning prior values and distributions to each of the two or more treatment regimens, wherein the prior values comprise a mean toxicity rate and a mean efficacy rate, wherein the distributions comprise a credible intervals for the mean toxicity rate and credible intervals for the mean efficacy rate;
allocating a population of subjects to the two or more different treatment regimens;
assessing and/or monitoring the subjects for toxicity and response events for a period of time that begins when the first dose of the therapeutic agent is administered to the subjects in the treatment regimen;
updating the prior values and calculating a utility score for each of the two or more treatment regimens based on toxicity and response events that are observed during the period of time in subjects who have completed one of the two or more treatment regimens;
identifying an optimal treatment regimen based on the utility scores calculated for each of the two or more treatment regimens; wherein the optimal treatment regimen has acceptable efficacy and toxicity rates.
